(19) United States
(12) Patent Application Publication
(10) Pub. No.: US 2024/0190937 A1
(43) Pub. Date: Jun. 13, 2024

(54) HUMAN INSULIN C-ALPHA-PEPTIDES AND METHODS OF USE

(71) Applicant: The United States of America, as represented by the Secretary, Dept. of Health and Human Services, Bethesda, MD (US)

(72) Inventors: Qing-Rong Liu, Perry Hall, MD (US); Min Zhu, Phoenix, MD (US); Pingbo Zhang, Columbia, MD (US); Josephine M. Egan, Bethesda, MD (US)

(73) Assignee: The United States of America, as represented by the Secretary, Dept. of Health and Human Services, Bethesda, MD (US)

(21) Appl. No.: 18/287,630

(22) PCT Filed: Apr. 21, 2022
(86) PCT No.: PCT/US2022/025764

§ 371 Date: Oct. 19, 2023

Related U.S. Application Data
(60) Provisional application No. 63/178,083, filed on Apr. 22, 2021.

(51) Int. Cl. C07K 14/62; A61K 45/06; A61P 3/00; A61P 25/28; G01N 33/68

(52) U.S. Cl. CPC C07K 14/62; A61K 45/06; A61P 3/00; A61P 25/28; G01N 33/6896; G01N 2333/62; G01N 2800/044; G01N 2800/2821

(57) ABSTRACT

Methods of treating a subject with diabetes or Alzheimer's disease with a disclosed insulin isoform or Cα polypeptide are provided. Methods of detecting insulin isoforms or Cα polypeptides are also provided.

### CROSS REFERENCE TO RELATED APPLICATION

This application claims the benefit of U.S. Provisional Application No. 63/178,083, filed Apr. 22, 2021, which is incorporated by reference herein in its entirety.

### FIELD

The present disclosure relates to human insulin isoforms and C-peptides and methods of their use, particularly for treating amyloidosis.

### BACKGROUND

The insulin gene (INS) underwent significant variations during evolution as its tissue expression migrated from neurons of invertebrates to islets of Langerhans in pancreas of vertebrates, and the functional spectrum of its peptide products diversified over time from growth and development, learning-memory, and reproduction, to primarily metabolism and regulation of anabolism, including its well-known function of regulating cellular uptake of glucose (Smit et al., Nature 331:535-538, 1988). In the central nervous system, the functions of insulin seem to more closely resemble actions that are attributed to ancestral insulins (Zeng et al., Neurol. Sci. 37:9-16, 2016). Insulin receptors are widely distributed throughout the brain where their downregulation is reported to be a feature of the neuropathology of Alzheimer's Disease (AD) (Griffith et al., Neuropharmacology 136:202-215, 2018). Furthermore, intranasal insulin administration is reported to improve cognitive function in people with mild cognitive impairment (MCI) and AD (Arnold et al., Nat. Rev. Neurol. 14:168-181, 2018). The primary source for brain insulin is presumed to be insulin secreted from β-cells in islets of Langerhans³. However, this is not the sole source because it is now established that human and mouse choroid plexus (CP) epithelium cells also express insulin. Mouse CP contains several Ins isoforms (Ins2-V1, -V2 and -V3) that are processed into mature insulin, which is then secreted into cerebrospinal fluid (CSF) proportional to CSF serotonin concentrations (Mazucanti et al., JCI Insight, 4:e131682, 2019).

Human INS in pancreatic β-cells is typically described as having three exons that encode a preproinsulin of 110 amino acids (AA) that undergoes extensive post-translational modifications. Proinsulin, a 24-AA signal peptide having been removed, exits the endoplasmic reticulum (ER) and is delivered to the Golgi apparatus for packaging in secretory vesicles. As vesicles become mature and acidify, proinsulin is typically then cleaved by protein convertases PC2 and/or PC1/3 then followed by carboxypeptidase E, freeing a 30-AA B-chain, a 31-AA C-peptide, and a 21-AA A-chain. In the process four amino acids (RR and RK) are lost from the ends of C-peptide. Secretory granules in 3 cells therefore contain various proportions of proinsulin, partially processed insulin, and mature insulin. Biologically active human and mouse insulin in circulation is composed of single A- and B-chains connected by disulfide bonds and small amounts of cleaved and non-cleaved proinsulin with some biological activity (Peavy et al., Diabetes 33:1062-1067, 1984).

Humans possess one INS gene while mice have two—Ins1 and Ins2. Ins1 is a retrogene derived from partially spliced Ins2 (Shiao et al., Genetics 178:1683-1691, 2008). Mouse islets express both genes in a 3:2 ratio (Leroux et al., Diabetes 50:S150-153, 2001), but only Ins2, the evolutionary older gene, is present in CP, while its expression is barely if at all detectable in other brain areas where Ins1 not at all detected (Mazucanti et al., JCI Insight, 4:e131682, 2019). Invertebrate genomes encode multiple insulin-related peptides in neurons, including pro-MIPs (molluscan insulin-related peptides) and post-translational processing produces A- and B-chains, and long and short C-peptides (Smit et al., Prog. Neurobiol. 54:35-54, 1998).

Next generation sequencing (NGS-RNAseq) identifies hundreds of human-specific genes and thousands of human-specific alternatively spliced isoforms that are implicated in human brain development and phenotypic intelligence together with pleiotropic negative effects in human diseases (Li et al., PLoS Comput. Biol. 6:e1000734, 2010; Zhang et al., Mol. Biol. Evol. 34:2453-2468, 2017; Zhang et al., Curr. Opin. Genet. Dev. 29:90-96, 2014). Multiple INS mRNA isoforms, all of which encode the known 110-AA preproinsulin, are reported to be regulated by glucose in pancreatic islets (Evans-Molina et al., Diabetes 56:827-835, 2007), but interestingly insulin secretion from CP is not controlled by glucose, but by serotonin (Mazucanti et al., JCI Insight, 4:e131682, 2019).

### SUMMARY

At least 50% of human genes contain upstream open reading frames (uORFs) that add layers of gene regulation and protein products for functional diversity. As INS evolved, expansion of actions of its isoform products in islets, CP and brain areas may have resulted in divergence of post-translational products and divergence of functions. Described herein are newly identified human specific uORF isoforms, INSU1 (intron-1 retention), INSU2 (intron-1 and -2 retention), and a new exon-3 splicing variant (here designated INS3B) that encodes a short 19-AA Cα-peptide at both mRNA and protein levels.

Provided here are methods of treating a subject with diabetes or Alzheimer's disease, which include administering to the subject a composition comprising an insulin Cα peptide. Also provided are methods of inhibiting amyloidosis (such as pancreatic islet amyloidosis or brain amyloidosis) in a subject, which include administering a composition comprising an insulin Cα peptide to a subject in need thereof. In some examples, the amino acid sequence of the insulin Cα peptide includes or consists of the amino acid sequence of SEQ ID NO: 7 or SEQ ID NO: 8. In some examples, the Cα peptide includes one or more modifications (such as N-terminal acetylation, C-terminal amidation, PEGylation, cyclization, a C14-C18 fatty acid, caprylic acid, fusion with a vitamin, or a combination of two or more thereof). In some examples, the composition including the Cα peptide is administered to the subject parenterally, orally, or via a patch.

In particular embodiments, the subject has type 1 diabetes or type 2 diabetes. In some examples, the methods further include administering to the subject with diabetes one or more of an insulin, a biguanide, a thiazolidinedione, a sulfonylurea, an incretin, and a sodium glucose co-transporter 2 inhibitor. In other embodiments, the subject has Alzheimer's disease and in some examples, the methods further include administering to the subject with Alzheimer's disease one or more of galantamine, rivastigmine, donepezil, memantine, and aducanumab.

Also provided herein are sets of peptides which may be used for detection and/or quantitation of insulin peptide, for example by mass spectrometry methods. In some embodiments, the set of peptides includes one or more peptides selected from the group consisting of the amino acid sequences of SEQ ID NOs: 54-64. In some examples, one or more of the peptides includes carbamidomethylation of one or more cysteine residues. In additional examples, one or more of the peptides includes an isotope label on one or more amino acids. In one non-limiting example, the set of peptides includes each of SEQ ID NOs: 54-64.

In additional embodiments, methods of detecting presence or amount of one or more insulin isoforms or Cα peptides in a sample are provided. In some examples, the methods include treating the sample with one or more enzymes to digest proteins in the sample; desalting the treated sample; and analyzing the treated sample by tandem mass spectrometry. In some examples, treating the sample with one or more enzymes to digest proteins comprises treating the sample with trypsin. In particular examples, the tandem mass spectrometry is liquid chromatography-mass spectrometry-mass spectrometry (LC-MS-MS). In some examples, the sample is a biological sample from a subject (such as blood, plasma, serum, CSF, saliva, urine, or tissue). In some examples, the subject has, is suspected to have, or is at risk of having pre-diabetes, diabetes, or Alzheimer's disease.

In some embodiments, the methods include identifying the subject as having type 2 diabetes if the amount of Cα peptide in the sample is increased compared to a control. In further embodiments, the methods include administering to the subject identified as having type 2 diabetes one or more of an insulin Cα peptide, an insulin, a biguanide, a thiazolidinedione, a sulfonylurea, an incretin, or a sodium glucose co-transporter 2 inhibitor.

In other embodiments, the methods include identifying the subject as having Alzheimer's disease if the amount of INSU2 (SEQ ID NO: 2) is increased compared to a control. In some examples, the methods further administering to the subject identified as having Alzheimer's disease one or more of an insulin Cα peptide, galantamine, rivastigmine, donepezil, memantine, and aducanumab.

Also provided are modified insulin Cα peptides. In some embodiments, the modified insulin Cα peptide includes the amino acid sequence of SEQ ID NO: 7 and SEQ ID NO: 8 and one or more peptide modifications. In some examples, the peptide modification includes one or more of N-terminal acetylation, C-terminal amidation, PEGylation, cyclization, a C14-C18 fatty acid, caprylic acid, fusion with a vitamin, and a combination of two or more thereof.

The foregoing and other features of the disclosure will become more apparent from the following detailed description, which proceeds with reference to the accompanying figures.

### BRIEF DESCRIPTION OF THE DRAWINGS

FIG. 1 is a schematic diagram of human INS gene structures and their alternatively spliced isoforms. uTSS, upstream transcription start site; pTSS, primary transcription start site; uORF, upstream open reading frame; pORF, primary open reading frame. Open boxes represent exons and solid lines represent introns. Downward arrows and capital letters are at the intra-exonal splicing donor sites of exon-1, and acceptor sites of exon-3 for human and polyadenylation sites. Red and green asterisks are the translation initiation sites for uORF and pORF, respectively, while the red dot is at the translational stop codon. The blue bar is the EX2 TaqMan probe that hybridizes to all the isoforms, the red bars are the isoform specific probes, the green bar is EX2-3A probe that hybridizes to all the INS isoforms except INS3B and INSU2, and the purple bar is the probe unique to the INS3B isoform. Red and blue names are the respective novel non-spliced and spliced uORF isoforms. The purple name is intra-exon-3 spliced INS3B isoform while the green names are the respective spliced and intron-1 retention pORF isoforms.

FIGS. 2A-2C are alignments of INSU1 (SEQ ID NO: 1), INSU2 (SEQ ID NO: 2) (FIG. 2A); INSUA (SEQ ID NO: 3), INSUB (SEQ ID NO: 4), and INSUC (SEQ ID NO: 5) (FIG. 2B); and INS (insulin; SEQ ID NO: 69) and INS3B (SEQ ID NO: 6) (FIG. 2C) amino acid sequences and tryptic peptides (arrows indicate the tryptic cutting sites of lysine or arginine) for protein quantification by SRM-MS. The amino acid sequence numbers are on the right and the signal peptide sequences are underlined. Red amino acids are the intrinsically disordered protein regions (IDPRs); lysine (K) for potential NF glycated NF-carboxymethyllysine are in light blue; the amino acid sequence in italics is the 42-AA polypeptide of the frame-shifted INSU2 encoded by intron-2, a 37-AA region (in red) of which is an IDPR sequence; dashed lines represent missing AA of Cα-peptide and the trypsin cutting site K of CαK is in green color; shaded regions are the SRM-MS quantified peptides; exon-intron and exon-exon junctions are respectively marked with/and I, respectively.

FIGS. 3A-3K are MS/MS spectra from insulin peptides (see Table 3). FIG. 3A, pep-U1 (SEQ ID NO: 58); FIG. 3B, pep-U2 (SEQ ID NO: 59); FIG. 3C, pep-U3 (SEQ ID NO: 60); FIG. 3D, pep-U4 (SEQ ID NO: 61); FIG. 3E, pep-U5 (SEQ ID NO: 62); FIG. 3F, pep-UF (SEQ ID NO: 63); FIG. 3G, pep-US (SEQ ID NO: 64). FIG. 3H, pep-B; By using respective applied declustering potential (DP), collision energy (CE), collision cell exits potential (CXP), unique fragmentation patterns for the peptide were observed. FIG. 3I, pep-A. Underlined cysteine residues denote carbamidomethylation (Cys-CAM). FIG. 3J, pep-Cα; FIG. 3K, pep-CαK. For each panel, zigzag lines between amino acids represent peptide fragment transitions at the sequence level and the inset shows a set of coeluting transition peaks in plasma matrix confirming that the detected SRM signals do derive from this peptide and no interfering of co-eluting peaks were present.

FIGS. 4A-4E show TaqMan RT-qPCR of INS3A (EX2-3A probe) and INS3B (EX2-3B probe) in human islets using total EX2 (exon-2 specific probe present in every isoform) as a reference (FIG. 4A), INS pORF isoforms in control (CNT, n=13) and T2DM islets (n=9) using INS1B as a reference (FIG. 4B) INS pORF isoforms in human islets (n=13) and CP (n=15) (FIG. 4C), comparison of INS isoforms in CP samples from control (CNT), T1DM and T2DM (FIG. 4D), and droplet digital (dd) PCR quantification of INS uORF isoforms in islets, CP, MTG and MFG (FIG. 4E).

FIGS. 5A and 5B show droplet digital (dd)PCR quantification. FIG. 5A is a graph showing comparison of autoantigen expression in islets (ISL) and CP. FIG. 5B is a graph showing comparison of INS gene transcription and differentiation factors in ISL and CP.

FIGS. 6A-6D are confocal microscopy (40×) images of triplex RNAscope ISH in control (FIG. 6A), T1DM (FIG. 6B), and T2DM (FIG. 6C) choroid plexus, and human islets (20×, FIG. 6D). Red represents INSU, green TTR (transthyretin) and magenta IAPP (islet amyloid polypeptide) probes.

FIGS. 7A-7F show expression of peptides in AD. FIGS. 7A-7B show TaqMan RT-qPCR INSU2 compared with IGF1 expression in control (CNT) and AD MTG (FIG. 7A) and MFG (FIG. 7B) samples, normalized with EX2 probe in control. FIGS. 7C-D show SRM quantification of pep-U1 (FIG. 7C) and pep-U3 (FIG. 7D) in CNT and AD MTG at protein level. In addition, confocal microscopy (20×) images of duplex RNAscope ISH in control MTG (FIG. 7E) and AD MTG (FIG. 7F) sections are provided. INSU ISH signal was colocalized in NEUN (neuron marker) in AD and not control MTG samples. Green represents the INSU ISH probe, red represents NEUN probe, and yellow arrow indicates the overlapped INSU and NEUN ISH probes.

FIGS. 8A and 8B show RT-qPCR of INSU2 in control (CNT) and Huntington disease (HD) of MTG samples (FIG. 8A) and SRM-MS quantification of pep-UF in control (CNT) and Alzheimer's disease (AD) (FIG. 8B). LOQ of pep-UF was only present in AD. LOQ represents limit of quantification and LOD limit of detection.

FIGS. 9A-9D shows plasma glucose (G) concentration (FIG. 9A); SRM pep-A (A-chain) and pep-B measurement (B-chain) (FIG. 9B); SRM of processed pep-Cα (Cα) and non-processed pep-CαK (CαK) measurement (FIG. 9C); and ratio of CαK/Cα (ratio of 74-AA proinsulin surrogate to mature insulin surrogate) (FIG. 9D) after an overnight fast (FG) and 2-hour continuous intravenous glucose (2 hr-IVG) infusion (continuous glucose infusion to maintain steady-state FG level+98 mg/dL for 2 hr).

FIGS. 10A and 10B show quantification by SRM-MS of pep-A (A-chain), pep-B (B-chain) and (total) insulin by ELISA (FIG. 10A) and quantification by SRM-MS of processed pep-Cα and non-processed pep-CαK, and C-peptide by ELISA (FIG. 10V). Plasma was obtained after an overnight fast (FG) and after 2 hr-IVG 2-hour continuous intravenous glucose infusion (2 hours: fasting glucose+98 mg glucose). Asterisk represents significant differences by 2-way ANOVA.

FIGS. 11A and 11B show SRM-MS quantification of A-chain (FIG. 11A) and B-chain (FIG. 11B) in human islets of control (CNT) and T2DM samples. Y axis represents endogenous pep-A and pep-B transition peak areas normalized with human albumin tryptic peptides.

FIGS. 12A-12F show SRM relative quantification of pep-Cα (FIG. 12A), pep-CαK (FIG. 12B), and ratio of CαK/Cα in human control (CNT) and T2DM islets (FIG. 12C) and SRM relative quantification (normalized with albumin) of pep-Cα (FIG. 12D), pep-CαK (FIG. 12E), and ratio of pep-CαK/Cα (FIG. 12F) in control (CNT), T1DM and T2DM choroid plexus samples. Y-axis is a ratio of the endogenous tryptic peptide transition peaks normalized to albumin tryptic peptides.

FIGS. 13A and 13B show mouse Ins2 gene structures and their alternatively spliced isoforms (FIG. 13A). uTSS, upstream transcription start site; pTSS, primary transcription start site. Open boxes represent exons and solid lines represent introns. Downward arrows and capital letters are at exon-2 and polyadenylation sites. Green asterisk represents translation initiation codon and red dot stop codon. FIG. 13B shows TaqMan RT-qPCR of mouse Ins2 uTSS and pTSS isoforms, Ins2-V1 and Ins2-V4, in islets, PAN (pancreas), CTX (cortex) and CP (choroid plexus). Ins2-V1 isoform mRNA was found in mouse islets to be more than 10⁵-fold higher than in CP, and 10⁸-fold higher than Ins2-V4 but not found in CTX.

FIG. 14 is a graph showing SRM-MS quantification of pep-Us encoded by INS uORF isoforms in control (CNT) and T2DM islet samples. Y axis represents endogenous pep-U transition peak areas normalized with that of human albumin.

FIGS. 15A and 15B illustrate SRM peptide selection, optimization, and validation. FIG. 15A shows structural features of human preproinsulin (SEQ ID NO: 69) and identifies the location of three candidate peptides: 01, 32 and a for SRM. FIG. 15B shows a set of coeluting transition peaks, confirming that the detected SRM signals in skeletal muscle matrix do derive from the targeted peptides (left) and no interfering of co-eluting peaks were present. The log₂-log₂ linear regression curves contain a constant amount of QC plasma matrix, 100 fmol/μl SIS mixture and a variable amount of light-SIS peptide (light form) mixture spiked prior to desalting (right) with or without exogenous peptide assistance. Linearity was determined by linear regression between the peak area ratio (light/heavy) and absolute injected amount of light SISs within the reportable range and expressed as R², and three replicates were analyzed for each concentration point. LOQ were determined from the std curve defined as the lowest concentration calibrated at which discovery estimated by back-fitting data to the std curve was 100±20% and technical replicates CV was ≤20%.

FIGS. 16A and 16B show reproducibility of SRM-based quantitation analysis. The entire SRM workflow replicates of the pooled QC plasma sample either within a day (intra-assay, n=8) or a different day (inter-assay, n=8) were digested with Biomek NX workstation. After desalting, a mixture of concentration balanced SIS peptides was added at final concentration of 100 fmol/μl. For EPA-SRM, an additional mixture of exogenous peptides at a precisely known concentration were added (15.6 ng/ml for peptide β1, 15.9 ng/ml for peptide β2 and 15.7 ng/ml for peptide α). Variations in the entire SRM workflow were evaluated as coefficient of variation (CV) for individual digests with 3-6 transitions per each target peptide. FIG. 16A shows variations of relative quantitation value. A given peptide relative quantitation value was obtained by summing peak area ratio (light/heavy) from all target peptide transitions and then averaging over three technical runs. FIG. 16B shows variations of retention time.

FIGS. 17A and 17B show interpeptide correlation and comparison of insulin peptide quantitation between SRM and ELISA. FIG. 17A shows insulin peptides (β1, β2 and α) and related levels in the plasma from 22 healthy individuals determined by using EPA-SRM and (−)SRM. After logarithmic transformation, an internal relationship between two sets of insulin peptides were evaluated by Pearson correlation analysis. FIG. 17B illustrates that combined two β peptides based on a strong relationship with each other (r=0.82, p<0.0001), correlation of peptide β and peptide α quantitation between SRM and ELISA was evaluated by Pearson correlation analysis.

FIGS. 18A and 18B illustrate agreement between SRM and ELISA quantitation. Bland-Altman plots were drawn to assess the difference against the average of (−)SRM and ELISA measurements (FIG. 18A) as well as EPA-SRM and ELISA measurements (FIG. 18B), with 95% limits of agreement (gray lines) as the mean difference ±1.96 SD of difference.

FIGS. 19A-19C show SRM and ELISA measurements for insulin. To evaluate the quantification accuracy of EPA-SRM, insulin concentration from 22 healthy adult plasma samples (7 fasting and 15 nonfasting) were measured by both SRM and the commercially available ELISA assay. FIG. 19A shows blood glucose level determined by using a Beckman Glucose Analyzer II (Beckman, Fullerton, CA) and insulin level measured by ELISA; insulin R and a peptide concentration levels determined by (−)SRM (FIG. 19B) and by EPA-SRM (FIG. 19C). The boxes stretch from the 25 to the 75 percentiles; the line across the boxes indicates the median values; the lines stretching from the boxes indicate extreme values. Statistical significance is shown * (p<0.01), **(p<0.001); ***(p<0.0001) versus 2 hr. during OGTT by unpaired two-tailed Student's t-test.

FIGS. 20A-20C show inhibition of IAPP fibrillation by C- and Cα-peptides. FIG. 20A: Representative IAPP fibrillation dynamics in three replicates. FIG. 20B: One-way ANOVA (box-whisker plot) showed significant different inhibitory effects of C- and Cα-peptides (P 5 0.032) on IAPP fibrillation. FIG. 20C: Linear regression analysis showed significant different inhibitory effects of C- and Cα-peptides (P 5 0.002) on IAPP fibrillation. Area under the curve (AUC) calculated by trapezoid rule. IAPP, 50 mmol/L IAPP plus buffer; C-pep, 50 mmol/L IAPP plus 50 mmol/L C-peptide; and Cα-pep, 50 mmol/L IAPP plus 50 mmol/L Cα-peptide. *P<0.05. F.I., fluorescence intensity.

FIGS. 21A-21C show inhibition of β-Amyloid (1-42) fibrillation by C- (C-pep) and Cα-peptides (Cα-pep). FIG. 21A shows representative β-Amyloid fibrillation dynamics and inhibition in three replicates. FIG. 21B is a box-whisker plot on C- and Cα-pep inhibition of β-Amyloid fibrillation in time range of 60-240 min. FIG. 21C shows linear regression analysis on C- and Cα-pep inhibition of β-Amyloid fibrillation in time range of 60-240 min. Fluorescence intensity (F.I.) represents ThT binding to amyloid. AUC represents area under curve calculated by Trapezoid rule. Aβ(1-42) represents 50 μM β-Amyloid plus buffer, C-pep represents 50 μM β-Amyloid plus 50 μM C-pep, and Cα-pep represents 50 μM β-Amyloid plus 50 μM Cα-pep.

### SEQUENCE LISTING

Any nucleic acid and amino acid sequences listed herein or in the accompanying Sequence Listing are shown using standard letter abbreviations for nucleotide bases and amino acids, as defined in 37 C.F.R. § 1.822. In at least some cases, only one strand of each nucleic acid sequence is shown, but the complementary strand is understood as included by any reference to the displayed strand.

SEQ ID NO: 1 is the amino acid sequence of INSU1.

SEQ ID NO: 2 is the amino acid sequence of INSU2.

SEQ ID NO: 3 is the amino acid sequence of INSUA.

SEQ ID NO: 4 is the amino acid sequence of INSUB.

SEQ ID NO: 5 is the amino acid sequence of INSUC.

SEQ ID NO: 6 is the amino acid sequence of INS3B.

SEQ ID NO: 7 is the amino acid sequence of Cα peptide.

SEQ ID NO: 8 is the amino acid sequence of CαK peptide.

SEQ ID NOs: 9-53 are splicing junction specific TaqMan probe and primer sequences of human INS and mouse Ins2 isoforms.

SEQ ID NOs: 54-64 and 71 are insulin tryptic peptide sequences for SRM-MS assay.

SEQ ID NO: 65 is a consensus human Kozak ribosomal binding site.

SEQ ID NO: 66 is the amino acid sequence of the Cα-peptide.

SEQ ID NO: 67 is a 3-sheet forming motif.

SEQ ID NO: 68 is an intrinsically disordered protein region sequence.

SEQ ID NO: 69 is an exemplary human preproinsulin sequence.

SEQ ID NO: 70 is a human serum albumin tryptic peptide.

SEQ ID NO: 71 is an insulin tryptic peptide.

### DETAILED DESCRIPTION

The complications of T1DM include significant occurrences of heart and blood vessel disease, neuropathy, nephropathy, eye damage, foot damage, skin and mouth conditions, and pregnancy complications. Insulin and islet amyloid polypeptide (IAPP), which can form amyloid fibrils in the islets of Langerhans in diabetes type 2, are kept non-aggregated by charge-based interactions with C-peptide at defined stoichiometries. It is possible that the conformational stabilization of insulin and IAPP by C-peptide may also counterbalance their aggregational tendencies at the high peptide concentrations in the pancreatic β-cell secretory granules. The concentration imbalances of C-peptide, insulin, and IAPP from the hyperpeptidism early in T2DM patients and the insulin-only injections in T1DM patients may distort equilibria of these peptide interactions and promote protein aggregation. Additionally, the chaperone-like actions of C-peptide may increase bioavailability of insulin supplements given to T1DM patients and prevent the formation of insulin deposits.

As disclosed herein, novel isoforms of insulin have been identified in humans. In one example, the isoform (INS3B) includes a modified C-peptide (designated Cα peptide) compared to previously identified sequences. The conventional 86-AA proinsulin is pre-folded into its correct conformation in the Golgi apparatus, together with the C-peptide (Tsiolaki et al., Biopolymers 108, doi: 10.1002/bip.22882, 2017). The N-terminus of the 31-AA C-peptide (EAEDLQVGQ) acts as a chaperone to promote the correct insulin folding, while its C-terminus (EGSLQ) is known to bind the orphan G-protein coupled receptor GPR146 (Kolar et al., J. Intern Med. 281:25-40, 2017). Furthermore, its middle region (VELGGGPGAGSLQP) is an intrinsic disorder protein region (IDPR) with amyloidogenic propensity (Tsiolaki et al., Biopolymers 108, doi: 10.1002/bip.22882, 2017). The human 19-AA Cα-peptide has the same estimated isoelectric point as that of C-peptide (pI=3.34) but it is devoid of the IDPR in the acidic (pH=4.5) environment of secretary granules (Orci et al., Cell 49:865-868, 1987; Landreh et al., Horm Metab Res 45:769-773, 2013), therefore, it might serve as anti-amyloidogenic agent for IAPP (pI=8.59) and insulin (Wahren et al., Diabetes Res Clin Pract 107:309-319, 2015; Kolar et al., J. Intern Med. 281:25-40, 2017). Thus, the disclosed Cα peptides (Cα and CαK) may be used in methods for treating or inhibiting amyloidosis. In addition, the Cα peptides may be used in methods of treating diabetes (such as T1DM and T2DM) or complications thereof. Finally, the Cα peptides may be useful for treating or inhibiting AD.

### I. Terms

Unless otherwise noted, technical terms are used according to conventional usage. Definitions of common terms in molecular biology may be found in Lewin's Genes X, ed. Krebs et al., Jones and Bartlett Publishers, 2009 (ISBN 0763766321); Kendrew et al. (eds.), The Encyclopedia of Molecular Biology, published by Blackwell Publishers, 1994 (ISBN 0632021829); Robert A. Meyers (ed.), Molecular Biology and Biotechnology: a Comprehensive Desk Reference, published by Wiley, John & Sons, Inc., 1995 (ISBN 0471186341); and George P. Rédei, Encyclopedic Dictionary of Genetics, Genomics, Proteomics and Informatics, 3^{rd} Edition, Springer, 2008 (ISBN: 1402067534), and other similar references.

Unless otherwise explained, all technical and scientific terms used herein have the same meaning as commonly understood by one of ordinary skill in the art to which this disclosure belongs. The singular terms “a,” “an,” and “the” include plural referents unless the context clearly indicates otherwise. “Comprising A or B” means including A, or B, or A and B. It is further to be understood that all base sizes or amino acid sizes, and all molecular weight or molecular mass values, given for nucleic acids or polypeptides are approximate, and are provided for description.

Although methods and materials similar or equivalent to those described herein can be used in the practice or testing of the present disclosure, suitable methods and materials are described below. All publications, patent applications, patents, and other references mentioned herein are incorporated by reference in their entirety, as are the GenBank Accession numbers, as present in the database on Apr. 15, 2021. In case of conflict, the present specification, including explanations of terms, will control. In addition, the materials, methods, and examples are illustrative only and not intended to be limiting.

In order to facilitate review of the various embodiments of the disclosure, the following explanations of specific terms are provided:

Amyloidosis: A group of diseases or conditions characterized by accumulation of amyloid in one or more organs or tissues. Amyloid deposits, or “plaques,” are inert, but interfere physically with tissue or organ structure and/or function. Amyloidosis occurs in Alzheimer's Disease, which is associated with accumulation of β-amyloid peptide (Aβ) in the brain. It also occurs in diabetes, with accumulation of islet amyloid polypeptide (IAPP) in the pancreatic islets (referred to as “islet amyloidosis” in some instances). Other forms of amyloidosis include primary amyloidosis, secondary amyloidosis, and familial amyloidosis.

Alzheimer's Disease: (AD): A progressive brain disorder that occurs gradually and results in memory loss, behavioral and personality changes, and a decline in mental abilities. These losses are related to the death of brain cells and the breakdown of the connections between them. The course of this disease varies from person to person, as does the rate of decline. On average, AD patients live for 8 to 10 years after they are diagnosed, though the disease can last up to 20 years. AD advances by stages, from early, mild forgetfulness to a severe loss of mental function. At first, AD destroys neurons in parts of the brain that control memory, especially in the hippocampus and related structures. As nerve cells in the hippocampus stop functioning properly, short-term memory fails. AD also attacks the cerebral cortex, particularly the areas responsible for language and reasoning.

Diabetes: A group of metabolic diseases in which a subject has high blood sugar, either because the pancreas does not produce enough insulin, or because cells do not respond to the insulin that is produced. Type 1 diabetes (T1DM) results from the body's failure to produce insulin. This form is also called “insulin-dependent diabetes mellitus” (IDDM) or “juvenile diabetes.” T1DM is characterized by loss of insulin-producing R cells, leading to insulin deficiency. This type can be further classified as immune-mediated or idiopathic. Type 2 diabetes (T2DM) results from insulin resistance, a condition in which cells fail to use insulin properly, sometimes combined with an absolute insulin deficiency. This form is also called “non-insulin-dependent diabetes mellitus” (NIDDM) or “adult-onset diabetes.” Diabetes is characterized by recurrent or persistent hyperglycemia, and is diagnosed by demonstrating any one of fasting plasma glucose level ≥7.0 mmol/l (126 mg/dl); plasma glucose ≥11.1 mmol/l (200 mg/dL) two hours after a 75 g oral glucose load as in a glucose tolerance test; symptoms of hyperglycemia and casual plasma glucose ≥11.1 mmol/l (200 mg/dl); and glycated hemoglobin (Hb A1C)≥6.5%

Isolated: An “isolated” biological component, such as a nucleic acid or protein, has been substantially separated or purified away from other biological components in the environment (such as a cell) in which the component occurs, e.g., other chromosomal and extra-chromosomal DNA and RNA and/or proteins. Nucleic acids and proteins that have been “isolated” include nucleic acids and proteins purified by standard purification methods. The term also embraces nucleic acids and proteins prepared by recombinant expression in a host cell as well as chemically synthesized nucleic acids.

Pharmaceutically acceptable carrier: The pharmaceutically acceptable carriers (vehicles) useful in this disclosure are known to one of ordinary skill in the art. Remington: The Science and Practice of Pharmacy, The University of the Sciences in Philadelphia, Editor, Lippincott, Williams, & Wilkins, Philadelphia, PA, 21^{st} Edition (2005), describes compositions and formulations suitable for pharmaceutical delivery of one or more therapeutic compounds or molecules, such as one or more peptides alone or in combination with additional pharmaceutical agents.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents, and the like, for example sodium acetate or sorbitan monolaurate.

Purified: The term purified does not require absolute purity; rather, it is intended as a relative term. Thus, for example, a purified protein or nucleic acid preparation is one in which the protein or nucleic acid is more enriched than the protein or nucleic acid is in its original environment (e.g., within a cell). In one embodiment, a preparation is purified such that the protein or nucleic acid represents at least 50% of the total protein or nucleic acid content of the preparation. Substantial purification denotes purification from other proteins or cellular components. A substantially purified protein or nucleic acid is at least 60%, 70%, 80%, 90%, 95% or 98% pure. Thus, in one specific, non-limiting example, a substantially purified protein or nucleic acid is 90% free of other components.

Recombinant: A nucleic acid or protein that has a sequence that is not naturally occurring or has a sequence that is made by an artificial combination of two otherwise separated segments of sequence (e.g., a “chimeric” sequence). This artificial combination can be accomplished by chemical synthesis or by the artificial manipulation of isolated segments of nucleic acids, for example, by genetic engineering techniques.

Subject: A living multi-cellular vertebrate organism, a category that includes both human and veterinary subjects, including human and non-human mammals.

Treating or ameliorating a disease: “Treating” refers to a therapeutic intervention that decreases or inhibits a sign or symptom of a disease or pathological condition after it has begun to develop, such as a reduction in tumor size or tumor burden. “Ameliorating” refers to the reduction in the number or severity of signs or symptoms of a disease, such as diabetes or Alzheimer's disease.

### II. Insulin Isoforms and Cα Peptides

Disclosed herein are newly identified human insulin isoforms and variant Cα peptides. In some embodiments, these isoforms and/or Cα peptides are isolated or purified. Exemplary amino acid sequences of the human insulin isoforms and Cα peptides are provided herein. Further provided are nucleic acid molecules (such as isolated nucleic acid molecules) encoding the human insulin isoforms and Cα peptides disclosed herein.

### A. Insulin Isoforms

Newly identified isoforms of human insulin are provided. In some embodiments, the human insulin isoforms are isolated or purified. In some examples, the isoforms are transcribed from a transcription start site upstream (5′) of the previously identified transcription start site (primary transcription start site, pTSS) and are referred to as being upstream open reading frames (uORFs). In other embodiments, the isoforms are alternatively spliced versions of the primary open reading frame (pORF).

In one embodiment, the human insulin isoform is an insulin uORF isoform referred to as INSU1 and has at least 95% sequence identity to (such as at least 96%, 97%, 98%, or 99% identity to) or includes or consists of the amino acid sequence:

(SEQ ID NO: 1)
MGSETIKPAGAQQPSALQDRLHQKRPSSRSVPRAFASGGLRIPGWLDPRP
QLCSREDVAGLVKHVGVSPGAPRQGTWPSACLSPACLPDHCPSAMALWMR
LLPLLALLALWGPDPAAAFVNQHLCGSHLVEALYLVCGERGFFYTPKTRR
EAEDLQVGQVELGGGPGAGSLQPLALEGSLQKRGIVEQCCTSICSLYQLE
NYCN

In another embodiment, the human insulin isoform is an insulin uORF isoform referred to as INSU2 and has at least 95% sequence identity to (such as at least 96%, 97%, 98%, or 99% identity to) or includes or consists of the amino acid sequence:

(SEQ ID NO: 2)
MGSETIKPAGAQQPSALQDRLHQKRPSSRSVPRAFASGGLRIPGWLDPRP
QLCSREDVAGLVKHVGVSPGAPRQGTWPSACLSPACLPDHCPSAMALWMR
LLPLLALLALWGPDPAAAFVNQHLCGSHLVEALYLVCGERGFFYTPKTRR
EAEDLQGEPTAHCCPWPPPATPCSWRSHPAWAEGGRRLPPSRGSGALF

In another embodiment, the human insulin isoform is an insulin uORF isoform referred to as INSUA and has at least 95% sequence identity to (such as at least 96%, 97%, 98%, or 99% identity to) or includes or consists of the amino acid sequence:

(SEQ ID NO: 3)
MGSETIKPAGAQQPSALQDRLHQKRPSSRSLSFCHGPVDAPPAPAGAAGP
LGT

In another embodiment, the human insulin isoform is an insulin uORF isoform referred to as INSUB and has at least 95% sequence identity to (such as at least 96%, 97%, 98%, or 99% identity to) or includes or consists of the amino acid sequence:

(SEQ ID NO: 4)
MGSETIKPAGAQQPSALQDRLHQKRPSSRSVPRAFASDHCPSAMALWMRL
LPLLALLALWGPDPAAAFVNQHLCGSHLVEALYLVCGERGFFYTPKTRRE
AEDLQVGQVELGGGPGAGSLQPLALEGSLQKRGIVEQCCTSICSLYQLEN
YCN

In another embodiment, the human insulin isoform is an insulin uORF isoform referred to as INSUC and has at least 95% sequence identity to (such as at least 96%, 97%, 98%, or 99% identity to) or includes or consists of the amino acid sequence:

(SEQ ID NO: 5)
MGSETIKPAGAQQPSALQDRLHQKRPSSRSVPRAFASGGLRIPGWLDPRS
LSFCHGPVDAPPAPAGAAGPLGT

In another embodiment, the human insulin isoform is an alternatively spliced insulin pORF isoform referred to as INS3B and has at least 95% sequence identity to (such as at least 96%, 97%, 98%, or 99% identity to) or includes or consists of the amino acid sequence:

(SEQ ID NO: 6)
MALWMRLLPLLALLALWGPDPAAAFVNQHLCGSHLVEALYLVCGERGFFY
TPKTRREAEDLQGSLQPLALEGSLQKRGIVEQCCTSICSLYQLENYCN

### B. Cα Peptides

Also provided are newly identified C peptides, referred to herein as Cα peptides. In some embodiments, the Cα peptides are isolated or purified.

In one embodiment, the Cα peptide has at least 95% sequence identity to (such as at least 96%, 97%, 98%, or 99% identity to) or includes or consists of the amino acid sequence:

| (SEQ ID NO: 7) | |
|---|---|
| | EAEDLQGSLQPLALEGSLQ |

In another embodiment, the Cα peptide has at least 95% sequence identity to (such as at least 96%, 97%, 98%, or 99% identity to) or includes or consists of the amino acid sequence:

EAEDLQGSLQPLALEGSLQK (CaK peptide; SEQ ID NO: 8)

In additional embodiments, the Cα peptides disclosed herein include one or more modifications, which is some examples may increase stability of the peptide (for example, resistance to proteolysis), solubility, and/or provide other desirable properties (such as improving tissue penetration or penetration of blood-brain barrier). While particular N- or C-terminal modifications are discussed below, each of the modifications may be at the N-terminus of the peptide, the C-terminus of the peptide, or both. In some examples, a Cα peptide is modified to include an N-terminal acetyl group and/or a C-terminal amide. In additional examples, the Cα peptide is modified with a polyethylene glycol (PEG) moiety (e.g., is a pegylated peptide). In other examples, the modifications include one or more N-methyl amides in the linear portion of the peptide. In yet further examples, a carbon chain (such as a 1-8 carbon chain) is added at the C-terminus of the Cα peptide. In still further examples, an aminoalkyl thiol (such as a C1-C8 alkyl) is added to the C-terminus of the peptide. In additional examples a Cα peptide is cyclized. In some embodiments, the cyclization is by head to tail amino and carboxyl link (and in some examples amino acid side chain protection and deprotection before and after the cyclization, respectively). In still further examples, a modified Cα peptide includes N-terminal caprylic acid (sodium N-(8-[2-hydroxybenzoyl]amino) or an N-terminal fatty acid (such as C14-C18-fatty acid). In another example, a Cα peptide is conjugated to vitamin B12, for example, by fusion of succinic acid modified N-terminal Cα-peptide to hydroxyl group of vitamin B12 (see, e.g., Petrus et al., Chem. Med. Chem. 4:421-426, 2009). Modified peptides can be synthetically produced.

In some examples, the disclosed Cα peptides may be in the form of one or more pharmaceutically acceptable salts or esters. Pharmaceutically acceptable salts include those formed from cations such as sodium, potassium, aluminum, calcium, lithium, magnesium, zinc, and from bases such as ammonia, ethylenediamine, N-methyl-glutamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)aminomethane, and tetramethylammonium hydroxide. The salts may be prepared by standard procedures, for example by reacting the free acid with a suitable organic or inorganic base. Representative bases include ammonium hydroxide, sodium hydroxide, potassium hydroxide, lithium hydroxide, calcium hydroxide, magnesium hydroxide, ferrous hydroxide, zinc hydroxide, copper hydroxide, aluminum hydroxide, ferric hydroxide, isopropylamine, trimethylamine, diethylamine, triethylamine, tripropylamine, ethanolamine, 2-dimethylaminoethanol, 2-diethylaminoethanol, lysine, arginine, histidine, and the like. Description of suitable pharmaceutically acceptable salts can be found in Handbook of Pharmaceutical Salts, Properties, Selection and Use, Wiley VCH (2002).

Pharmaceutically acceptable esters include, but are not limited to, methyl, ethyl, propyl, butyl, pentyl, hexyl, cyclopropyl, cyclobutyl, cyclopentyl, cyclohexyl, cycloheptyl, cyclopentenyl, cyclopentadienyl, cyclohexenyl, cyclohexadienyl, phenyl, pyridinyl, benzyl, and the like.

Pharmaceutically acceptable esters can be prepared by, for example, by treating the compound with an appropriate amount of carboxylic acid, ester, acid chloride, acid anhydride, or mixed anhydride agent that will provide the corresponding pharmaceutically acceptable ester. Typical agents that can be used to prepare pharmaceutically acceptable esters include, for example, acetic acid, acetic anhydride, acetyl chloride, benzylhalide, benzaldehyde, benzoylchloride, methyl ethylanhydride, methyl phenylanhydride, methyl iodide, and the like.

### C. Tryptic Peptides

Also provided are tryptic peptides of the disclosed insulin isoforms and Cα peptides. In particular embodiments, the tryptic peptides include or consist of the amino acid sequences shown in Table 3. In some examples, the tryptic peptides are provided as a set of one or more (such as 1, 2, 3, 4, 5, 6, 7, 8, 9, 10, 11, 12, 13, 14, 15, 16, 17, 18, 19, 20, 21, or 22) of the peptides of Table 3. In one non-limiting example, the set of peptides includes or consists of Pep-A (SEQ ID NO: 55), Pep-B (SEQ ID NO: 54), Pep-Cα (SEQ ID NO: 56), Pep-CαK (SEQ ID NO: 57), and Pep-UF (SEQ ID NO: 64). One or more of the peptides maybe modified by carbamidomethylation of one or more cysteine residues and/or inclusion of an isotope label (e.g., deuterium, carbon-13, and/or nitrogen-15) on one or more amino acids. As discussed below, the tryptic peptides can be used in methods of detecting the insulin isoforms or Cα peptides, for example, utilizing a mass spectrometry assay.

### III. Methods of Treating a Disease or Disorder

Provided herein are methods of treating a disease or disorder utilizing one or more of the disclosed insulin isoforms or Cα peptides. In particular embodiments, the methods include treating diabetes or Alzheimer's disease (AD). In some examples, the methods include treating or inhibiting complications of T1DM or T2DM. In other embodiments, the methods include inhibiting or decreasing amyloidosis, for example pancreatic islet amyloidosis (e.g., T2DM islet amyloidosis) or brain amyloidosis.

In one example, the methods include treating a subject with diabetes by administering to the subject an insulin Cα peptide disclosed herein. In another example, the methods include inhibiting or decreasing pancreatic islet amyloidosis in a subject by administering an insulin Cα peptide disclosed herein to the subject. In some non-limiting examples, the Cα peptide includes or consists of the amino acid sequence of SEQ ID NO: 7 or SEQ ID NO: 8. In additional examples, the Cα peptide includes one or more modifications, which in some non-limiting examples includes N-terminal acetylation, C-terminal amidation, cyclization, PEGylation, modification with caprylic acid, fatty acids, or vitamin B12.

In some examples, the Cα peptide is administered to a subject with diabetes in combination with mature insulin. For example, the subject may be administered a composition including mature insulin and a Cα peptide. In one example, the insulin and Cα peptide are administered at a molar 1:1 ratio. In other examples, the insulin and Cα peptide are administered at a molar 1:2, 1:3, 1:4, 1:5, 1:6, 1:7, 1:8, 1:9, or 1:10 ratio. However, other ratios of mature insulin and Cα peptide can be selected by one of ordinary skill in the art, for example, based on in vitro assays, pre-clinical experiments, and/or clinical trials.

In some embodiments, the subject has Type 1 diabetes (T1DM). In some examples, the subject has developed one or more complications of T1DM, such as diabetes-associated retinopathy, vasculopathy, nephropathy, and/or neuropathy. In other examples, the subject has been diagnosed with T1DM for at least 5 years, such as at least 5, 6, 7, 8, 9, 10 years or more. In particular examples, administering a Cα peptide of the disclosure (for example, in combination with mature insulin) to the subject treats or ameliorates one or more signs or symptoms of T1DM or a complication thereof in the subject.

In other embodiments, the subject has Type 2 diabetes (T2DM). In some examples, the subject has developed islet amyloidosis. In other examples, the subject with T2DM has begun treatment with insulin. In particular examples, administering a Cα peptide to the subject decreases or inhibits formation of amyloid plaques in pancreatic islets of the subject.

In other embodiments, the subject has Alzheimer's disease. In particular examples, administering a Cα peptide of the disclosure to the subject treats or ameliorates one or more signs or symptoms of Alzheimer's disease in the subject. For example, administering a Cα peptide to the subject in some examples decreases or inhibits formation of amyloid plaques (such as A3 plaques) in the brain of the subject. In some examples, the methods include treating a subject with AD by administering to the subject an insulin Cα peptide disclosed herein. In another example, the methods include inhibiting or decreasing brain amyloidosis in a subject by administering an insulin Cα peptide disclosed herein to the subject. In some non-limiting examples, the Cα peptide includes or consists of the amino acid sequence of SEQ ID NO: 7 or SEQ ID NO: 8. In additional examples, the Cα peptide includes one or more modifications, which in some non-limiting examples includes N-terminal acetylation, C-terminal amidation, cyclization, PEGylation, modification with caprylic acid, fatty acids, or vitamin B12. In some examples, a modification that increases the ability of the peptide to cross the blood-brain barrier is selected.

The disclosed peptides can be administered by any means known to one of skill in the art, such as by intramuscular, subcutaneous, intraperitoneal, or intravenous injection, but even oral, nasal, or anal administration is contemplated. The disclosed peptides can also be administered topically, transdermally, or by local injection. In some embodiments, administration is orally, by intravenous injection, or subcutaneous injection. To extend the time during which the peptide is available, the peptide can be provided as an implant, an oily injection, or as a particulate system. The particulate system can be a microparticle, a microcapsule, a microsphere, a nanoparticle, a nanocapsule, or similar particle. In other examples, the peptide is present in an absorbable matrix, such as an absorbable polymer (e.g., polylactide, polyglycolide, polylactide glycolide, etc.). One of ordinary skill in the art is aware of methods of administering peptides to a subject. See, e.g., Banga, “Parenteral Controlled Delivery of Therapeutic Peptides and Proteins,” in Therapeutic Peptides and Proteins, Technomic Publishing Co., Inc., Lancaster, P A, 1995. They may be administered in different forms, including but not limited to solutions, emulsions and suspensions, microspheres, particles, microparticles, nanoparticles, and liposomes.

In some examples, the provided peptides are combined with a pharmaceutically acceptable carrier or vehicle for administration to human or animal subjects. Examples of suitable pharmaceutically acceptable carriers, vehicles, or excipients include sterile aqueous or non-aqueous solutions, suspensions, and/or emulsions. Examples of non-aqueous solvents include propylene glycol, polyethylene glycol, vegetable oils such as olive oil, and injectable organic esters such as ethyl oleate. Aqueous carriers include water, alcoholic/aqueous solutions, emulsions, or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

In another embodiment, it may be desirable to administer the peptides or pharmaceutical compositions locally to the area in need of treatment. This may be achieved by, for example, and not by way of limitation, local or regional infusion or perfusion, topical application, injection, catheter, suppository, or implant (e.g., implants formed from porous, non-porous, or gelatinous materials, including membranes, such as sialastic membranes or fibers), and the like. In other embodiments, the peptides or compositions are administered using a patch, such as a transdermal microneedle patch (see, e.g., Wang et al., J. Mater. Chem. B 8:9335-9342, 2020).

The peptides can be conveniently presented in unit dosage form and prepared using conventional pharmaceutical techniques. Such techniques include the step of bringing into association the active ingredient and the pharmaceutical carrier(s) or excipient(s). In general, the formulations are prepared by uniformly and intimately bringing into association the active ingredient with liquid carriers. Formulations suitable for parenteral administration include aqueous and non-aqueous sterile injection solutions which may contain anti-oxidants, buffers, bacteriostatic agents, and/or solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampoules and vials, and may be stored in a freeze-dried (lyophilized) condition requiring only the addition of a sterile liquid carrier, for example, water for injections, immediately prior to use. Injection solutions and suspensions may be prepared from sterile powders, granules and tablets commonly used by one of ordinary skill in the art.

In certain embodiments, unit dosage formulations are those containing a dose or unit, or an appropriate fraction thereof, of the administered ingredient. It should be understood that in addition to the ingredients particularly mentioned above, formulations encompassed herein may include other agents commonly used by one of ordinary skill in the art.

The amount of the peptide(s) that will be effective depends on the nature of the disorder or condition to be treated, as well as the stage of the disorder or condition. Effective amounts can be determined by standard clinical techniques. The precise dose of the peptide(s) to be employed in the formulation will also depend on the route of administration, and should be decided according to the judgment of the health care practitioner and each subject's circumstances. An example of such a dosage range is about 1 μg to 500 mg (such as about 1 μg to 100 kg, about 10 μg to 1 mg, about 100 μg to 2 mg, about 500 μg to 4 mg, about 750 μg to 5 mg, about 1 mg to 10 mg, about 5 mg to 25 mg, about 15 mg to 50 mg, about 30 mg to 75 mg, about 50 mg to 100 mg, about 75 mg to 250 mg, or about 100 mg to 500 mg in single or divided doses. Unit dosage forms are also possible, for example 0.01 mg, 0.05 mg, 0.1 mg, 0.25 mg, 0.5 mg, 1 mg, 1.5 mg, 2 mg, 2.5 mg, 3 mg, 4 mg, 5 mg, 10 mg, 15 mg, 20 mg, 25 mg, 50 mg, 100 mg, 150 mg, 200 mg, or up to 500 mg per dose. In some non-limiting examples, the dose is about 0.8 mg or 2.4 mg, for example, administered weekly. In other examples, the dosage range is about 0.001 mg/kg to 200 mg/kg (such as about 0.001 mg/kg to 0.005 mg/kg, about 0.005 mg/kg to 0.01 mg/kg, about 0.01 mg/kg to 0.025 mg/kg, about 0.025 mg/kg to 0.075 mg/kg, about 0.05 mg/kg to 0.5 mg/kg, about 0.25 mg/kg to about 1 mg/kg, about 0.5 mg/kg to about 2.5 mg/kg, about 1 mg/kg to about 5 mg/kg, about 5 mg/kg to about 10 mg/kg, about 10 mg/kg to about 25 mg/kg, about 20 mg/kg to about 50 mg/kg, about 25 mg/kg to about 75 mg/kg, about 50 mg/kg to about 100 mg/kg, about 75 mg/kg to about 150 mg/kg, or about 100 mg/kg to about 200 mg/kg). However, other higher or lower dosages and frequency of administration also could be used, as can be determined by in vitro and/or in vivo testing.

The specific dose level and frequency of dosage for any particular subject may be varied and will depend upon a variety of factors, including the activity of the specific compound, the metabolic stability and length of action of that compound, the particular disease or disorder to be treated, the age, body weight, general health, sex, diet, mode and time of administration, rate of excretion, and/or any drug combinations administered. Treatment can involve daily or multi-daily, weekly, bi-monthly, or monthly doses of compound(s) over a period of a few days or weeks to months, or even years.

The pharmaceutical compositions of the present disclosure can be administered at about the same dose throughout a treatment period, in an escalating dose regimen, or in a loading-dose regime (e.g., in which the loading dose is about two to five times the maintenance dose). In some embodiments, the dose is varied during the course of a treatment based on the condition of the subject being treated, the severity of the disease or condition, the apparent response to the therapy, and/or other factors as judged by one of ordinary skill in the art.

The disclosed peptides can be used alone or in combination therapy with other compositions or drugs used to treat the described conditions. In embodiments where the subject being treated has diabetes, one or more additional treatments for diabetes or diabetes-associated conditions may be administered, for example, one or more of insulin (such as an insulin isoform identified herein, and/or a different form of insulin from those disclosed herein), biguanides (such as metformin), thiazolidinediones (such as ciglitazone, pioglitazone, rosiglitazone, or lobeglitzone), sulfonylureas (such as glyburide, glimepiride, glibornuride, gliclazide, glipizide, gliquidone, glisoxepide, or glyclopyramide), incretins (such as exenatide, liraglutide, sitagliptin, saxagliptin, alogliptin, or linagliptin), and sodium glucose co-transporter 2 (SGLT2) inhibitors (such as canagliflozin, dapagliflozin, or empagliflozin). In embodiments, where the subject being treated has AD, one or more additional treatments for AD or AD-associated conditions may be administered, for example, one or more of galantamine, rivastigmine, donepezil, memantine, and aducanumab. A skilled clinician can select appropriate additional therapies for a subject being treated using the methods disclosed herein.

### IV. Methods of Detecting Insulin Isoforms

Also provided herein are methods of detecting insulin isoforms or Cα peptides, for example, in a sample from a subject. In some embodiments, the methods further include diagnosing the subject as having or being at increased risk of a disease or disorder, such as diabetes or Alzheimer's disease.

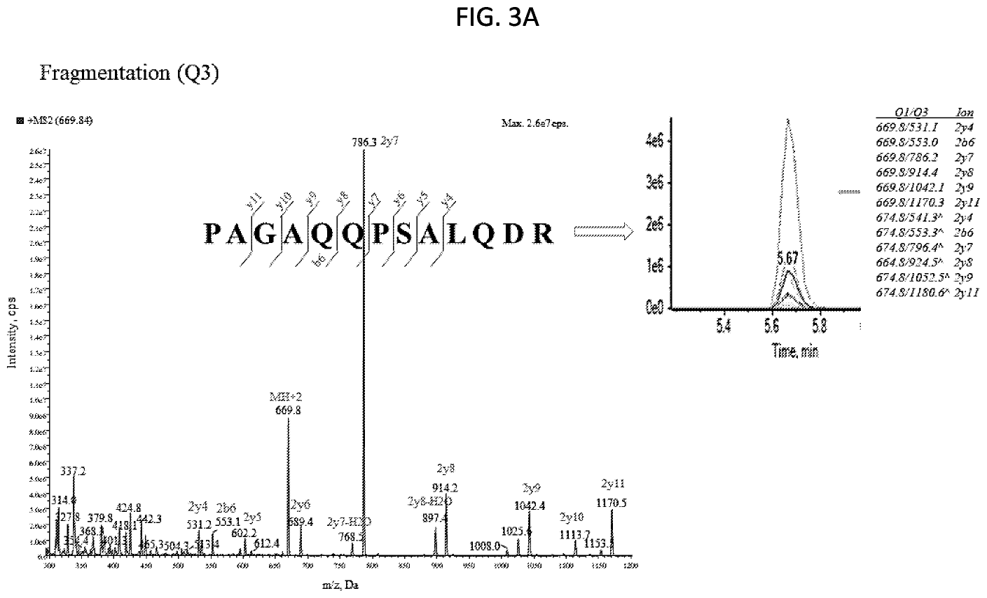

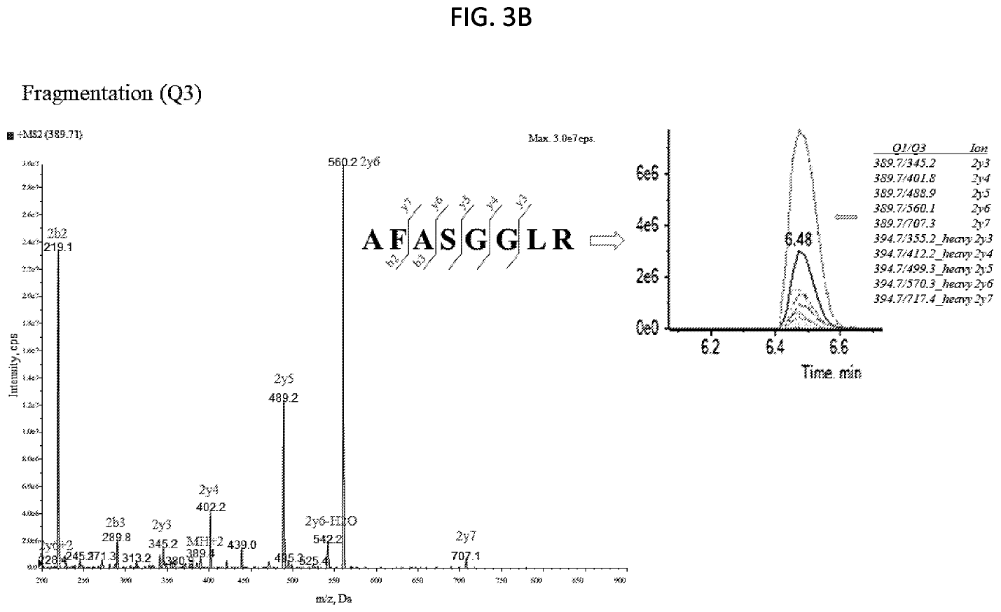

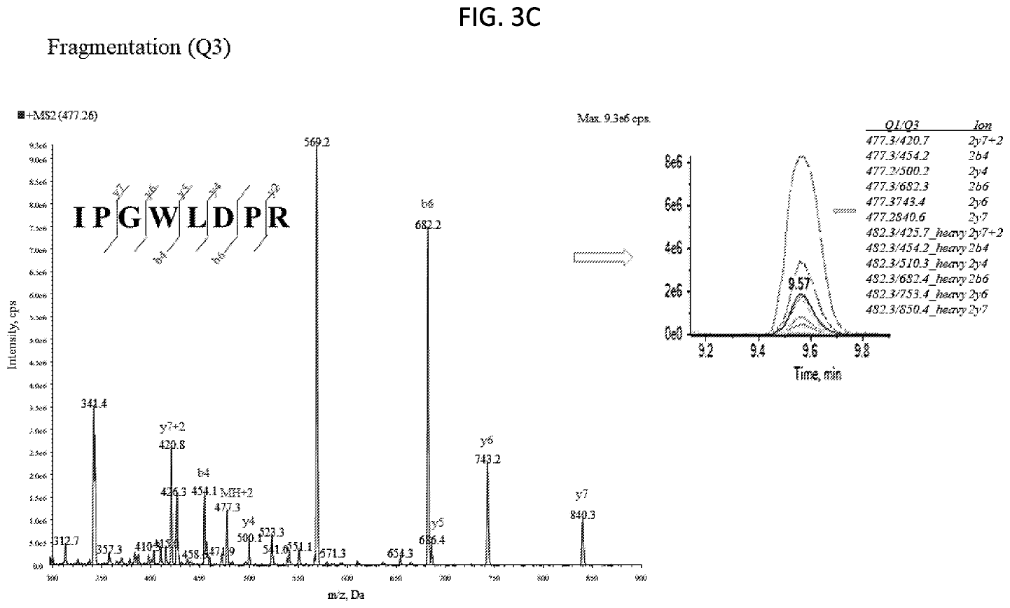

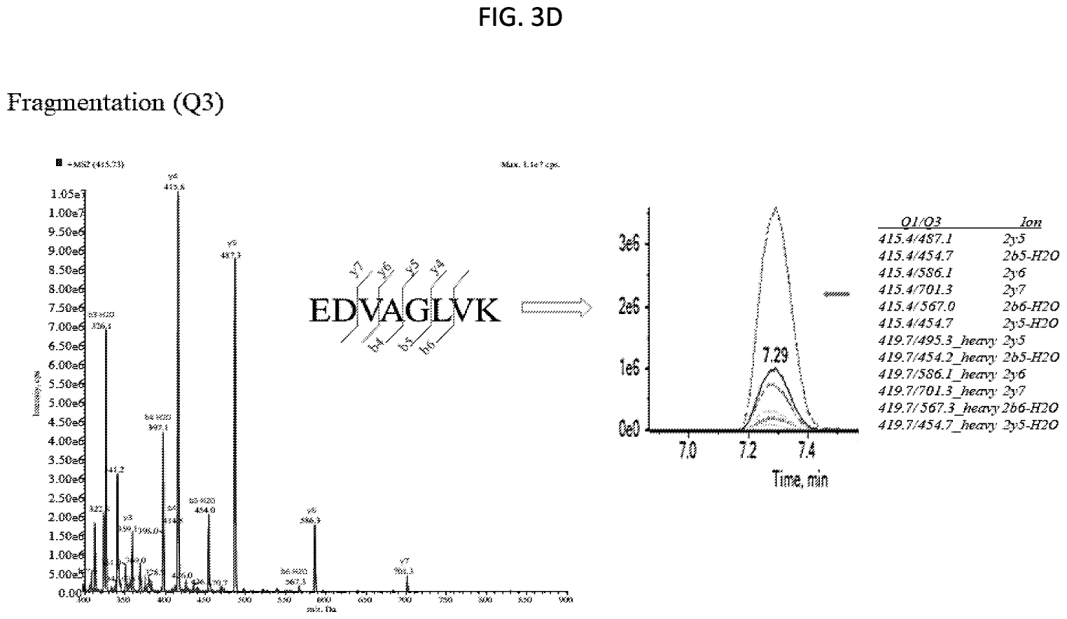

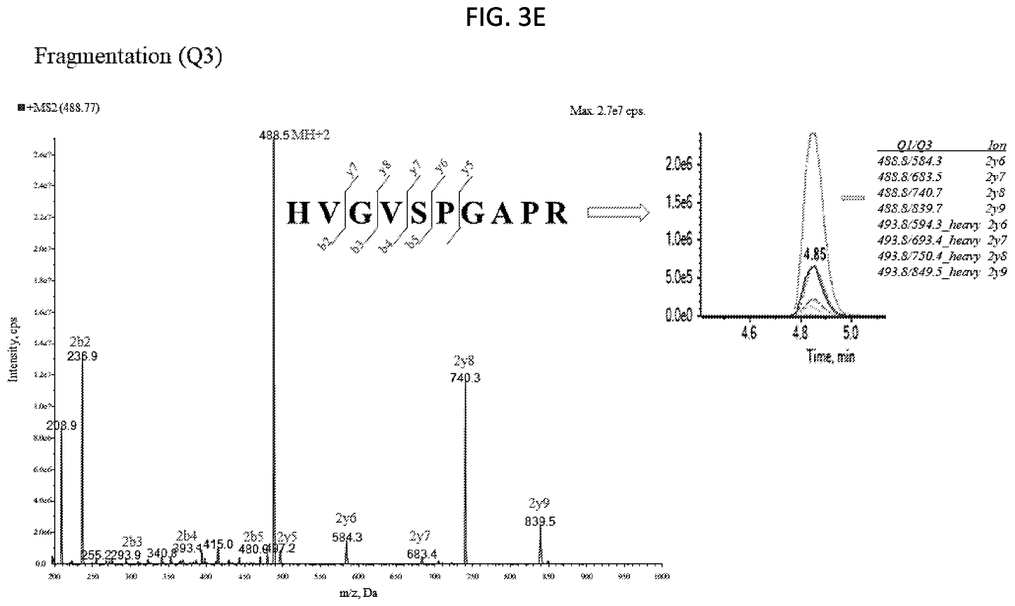

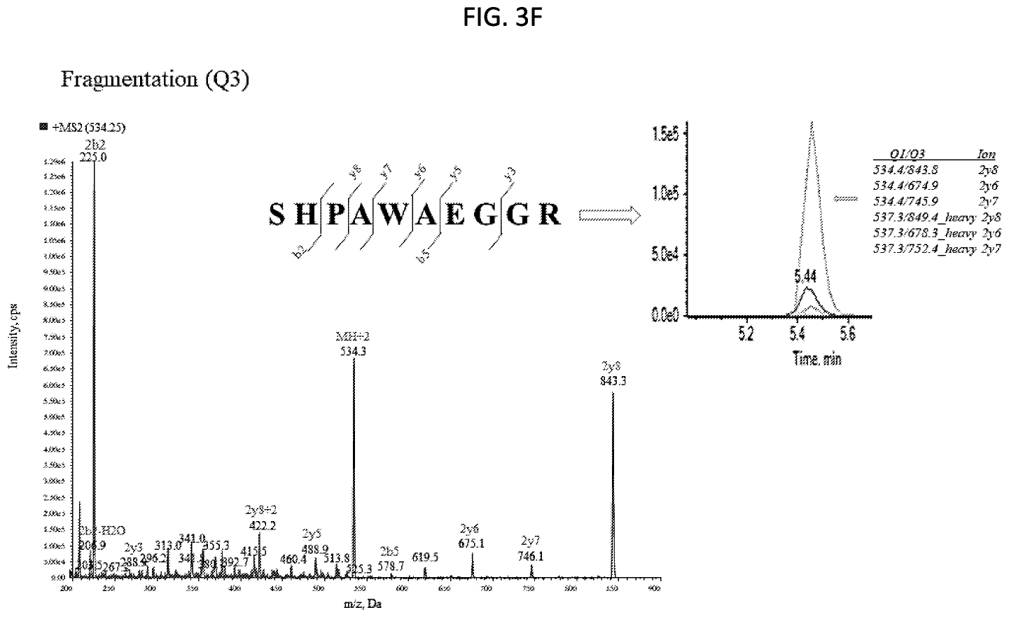

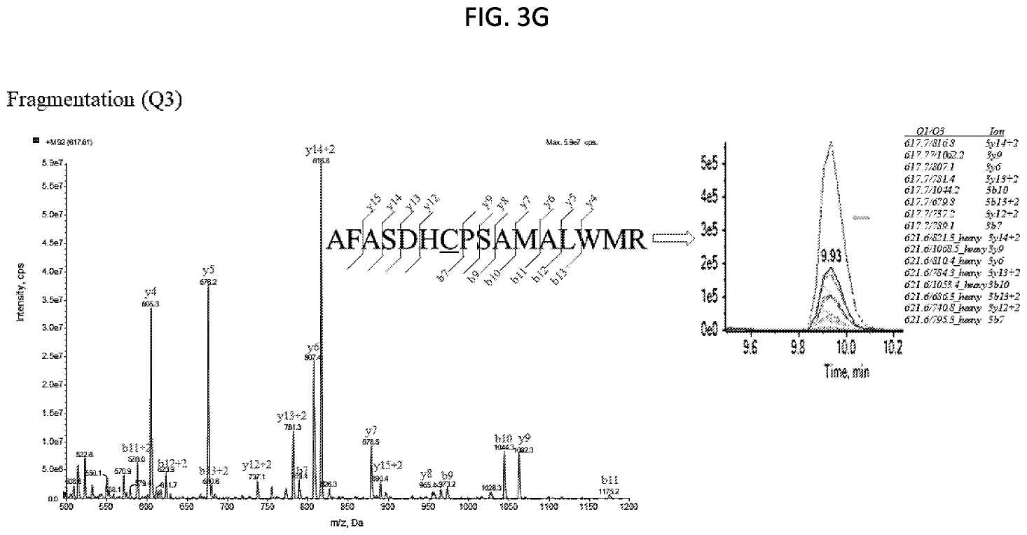

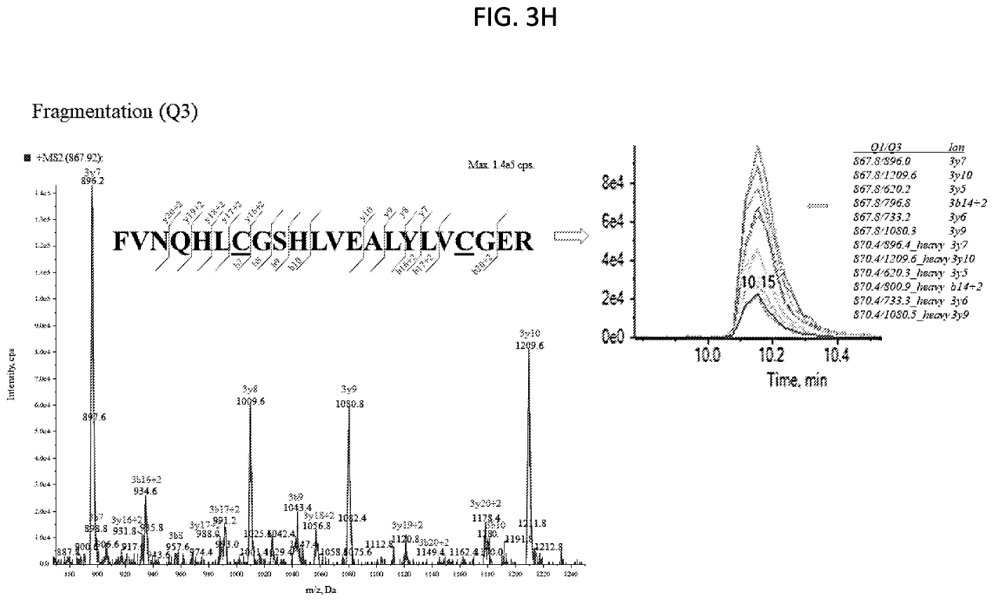

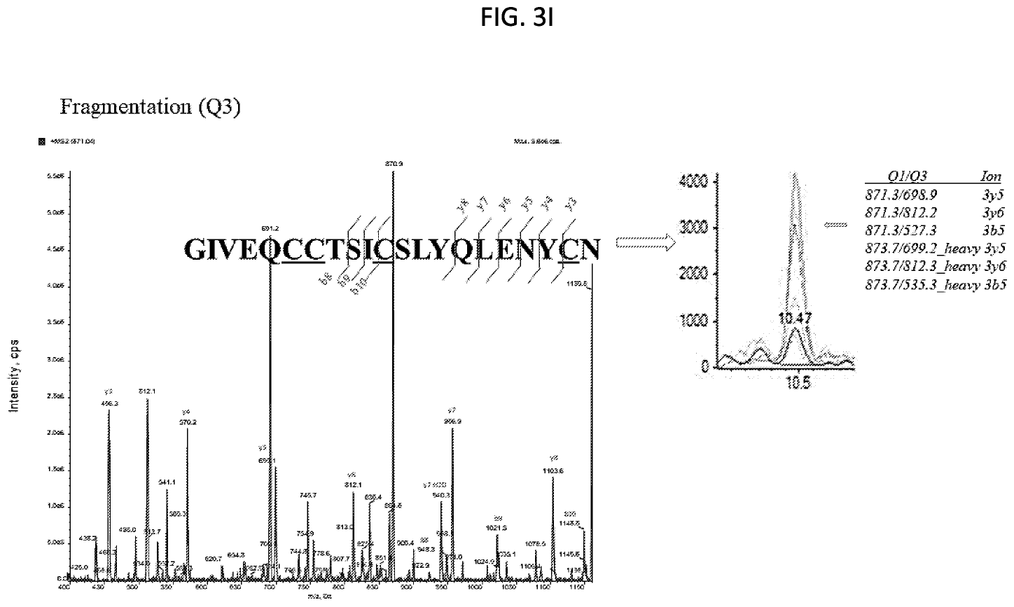

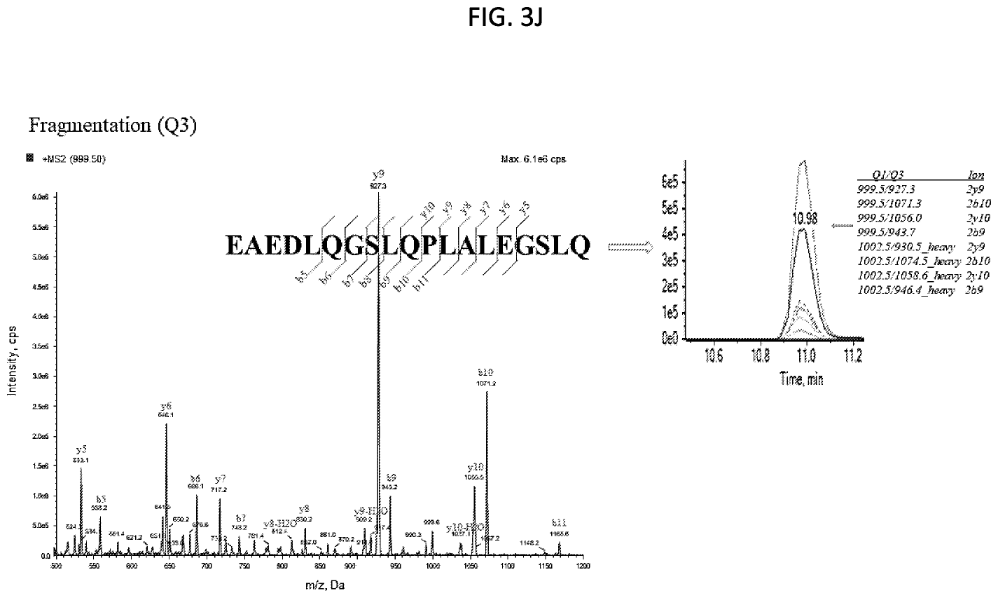

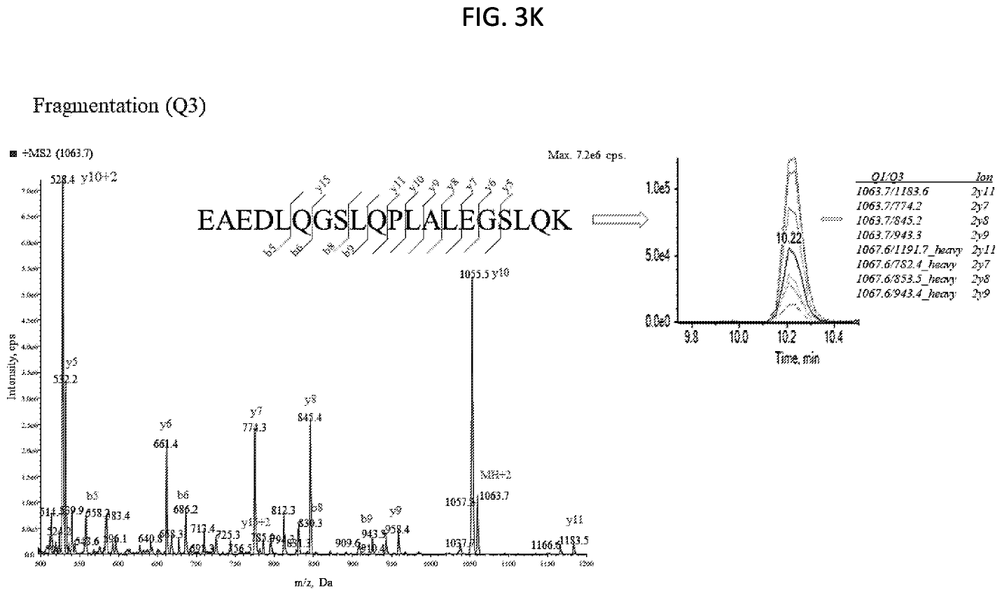

In some embodiments, the methods include detecting one or more of the disclosed insulin isoforms or Cα peptides using a tandem mass spectrometry (MS) assay. In some examples, the method is a selected reaction monitoring (SRM)-MS assay. In one non-limiting example, the methods include tryptic digestion of a sample followed by desalting. The resulting preparation is analyzed by liquid chromatography (LC)-MS-MS. Presence and amount of insulin isoforms and Cα peptides are determined based on the tryptic peptides provided in Table 3 (see also, FIGS. 3A-3K). In some examples, the amount of insulin isoform(s) or Cα peptide(s) in a sample is determined using a standard curve. Exemplary methods are described in Examples 1, 6, and 7.

In particular embodiments, the methods include SRM analysis of tryptic peptides. The methods include resuspending tryptic digests in 0.1% formic acid and mixing well. Stable isotope-labeled standard (SIS) peptides mixture is added to a final concentration to 100 fmol/l per each SIS peptide. The mixture is mixed well by vortex and then centrifuged (e.g., 14,000 rpm for 10 min at RT). SRM analysis is carried out. An exemplary instrument is a QTRAP 5500 mass spectrometer with a Shimadzu LC-HPLC equipped with LC-20ADXR pumps for solvent and sample delivery and a 2.1 mm×100 mm, 130 Å pore size, 3.5 m particle size C18 column for the peptide separations; however, other suitable instrumentation can be used. An exemplary linear gradient is: 0 min 5% B (ACN in 0.1% FA); 10 min 36% B; 12 min 90% B; 13.5 min 90% B; 14 min 5% B at a flow rate of 0.2 ml/min. The gradient can be extended for improved separation, depending on the samples. QTRAP 5500 mass spectrometer with electrospray ionization (ESI) source controlled by Analyst 1.5 software may be used for all LC-SRM-MS detection and analysis. Mass spectrometric analyses are performed in positive ion mode. ESI interface parameters may be set as follows: capillary temperature 650° C. and a curtain gas setting of 30 psi.

Analysis of SRM data is quite simple compared to most other mass spectrometric analysis. The output of SRM analysis is an elution profile (chromatogram of each SRM Q1/Q3 ion pair). Data analysis involves recognition and integration of the peak that is specific to the target peptides. An exemplary data analysis protocol is as follows:
1. Process the SRM data with assistance of MultiQuant 3.1 software using the scheduled SRM algorithm for peak integration
2. Default Peak integration settings to detect the endogenous peak based on the SIS peptide's retention time (RT).
3. Use a peak-splitting factor of 1 and default values for noise percentage and baseline subtraction window.
4. Manually inspect to ensure that the MultiQuant software recognized and integrated the correct peak for each SRM transition ion.
5. Work with ratios of endogenous peak are to SIS peptide peak are (light/heavy).
6. Sum all transition peak area ratio to get peak area ratio at the peptide level.
7. Output of MultiQuant for linear regression weighted by 1/χ2 to prevent higher values from over-contributing to the liner regression in a dilution series.

In some embodiments, sample preparation prior to assay includes reducing disulfide bonds in a sample (such as delipidated plasma or CSF sample), alkylation of free cysteines (for example using iodoacetic acid), and tryptic digestion. In some examples, the tryptic digests undergo solid phase extraction and drying prior to assay.

Although specific detection methods are provided herein, other methods of detecting the disclosed insulin isoforms and Cα peptides can also be used. These assays include, but are not limited to, ELISA, radioimmunoassay, or other MS methods.

The sample can be any type of specimen in which a disclosed insulin isoform or Cα peptide is present or can be present. In some embodiments, the sample is a biological specimen containing nucleic acids (for example, DNA, RNA, and/or mRNA), proteins, or combinations thereof, obtained from a subject. Examples include, but are not limited to, peripheral blood, serum, plasma, urine, saliva, cerebrospinal fluid, tears, sweat, exosomes, tissue, tissue biopsy, fine needle aspirate, surgical specimen, and autopsy material. In some examples, a sample includes blood, serum, plasma, or urine. In some embodiments, the sample is from a subject having diabetes or suspected of having diabetes or being at increased risk of diabetes (for example, a subject with pre-diabetes or metabolic syndrome). In other embodiments, the sample is from a subject having AD, or suspected of having or being at increased risk of AD.

In some embodiments, the amount of the insulin isoform and/or Cα peptide measured is compared to a control. A “control” refers to a sample or standard used for comparison with an experimental sample. In some embodiments, the control is a sample obtained from a healthy subject (such as a subject without diabetes or AD). In other embodiments, the control is a historical control or standard reference value or range of values (such as a previously tested control sample, such as a group of subjects with diabetes or AD, or group of samples from subjects that do not have diabetes or AD). In further examples, the control is a reference value, such as a standard value obtained from a population of normal individuals that is used by those of skill in the art. Similar to a control population, the value of the sample from the subject can be compared to the mean reference value or to a range of reference values (such as the high and low values in the reference group or the 95% confidence interval). In other examples, the control is the subject (or group of subjects) treated with placebo compared to the same subject (or group of subjects) treated with the therapeutic compound, for example, as in a cross-over study.

In some examples, the methods further include identifying a subject as having or being at risk of a condition, based on detecting presence and/or amount of one or more of the disclosed insulin isoforms or Cα peptides. In some examples, a subject is diagnosed as having diabetes (for example, T2DM) if the amount of Cα peptide is increased compared to a control. In other examples, a subject is diagnosed as having diabetes or being at risk of diabetes if the ratio of Cα peptide to CαK peptide is decreased compared to a control.

In other examples, a subject is diagnosed as having AD if the amount of INSU2 (e.g., SEQ ID NO: 2) is increased compared to a control (for example, compared to a subject or reference value that does not have AD). In some examples, the amount of INSU2 is detected using the amount of Pep-UF (SEQ ID NO: 64) in the detection assays described herein.

In some embodiments, the amount of the insulin isoform or Cα peptide is increased by about 10%, 20%, 30%, 40%, 50%, 60%, 70%, 80%, 90%, 1.2-fold, 1.5-fold, 2-fold, 3-fold, 4-fold, 5-fold, 10-fold, or more compared to the control. In other embodiments, the amount of the insulin isoform or Cα peptide is decreased by about 10%, 20%, 30%, 40%, 50%, 60%, 70%, 80%, 90%, or more compared to the control. In additional examples, the amount of the insulin isoform or Cα peptide is significantly different (for example, statistically significantly different) from the control.

In some embodiments, the disclosed methods further comprising treating a subject identified as having diabetes or AD. In particular examples, the subject is treated as described in Section III, above. In other examples, the subject is treated with one or more therapies for the identified disorder, such as one or more of insulin (such as an insulin isoform identified herein, and/or a different form of insulin from those disclosed herein), biguanides (such as metformin), thiazolidinediones (such as ciglitazone, pioglitazone, rosiglitazone, or lobeglitzone), sulfonylureas (such as glyburide, glimepiride, glibornuride, gliclazide, glipizide, gliquidone, glisoxepide, or glyclopyramide), incretins (such as exenatide, liraglutide, sitagliptin, saxagliptin, alogliptin, or linagliptin), and sodium glucose co-transporter 2 (SGLT2) inhibitors (such as canagliflozin, dapagliflozin, or empagliflozin) if the subject has diabetes, or one or more of galantamine, rivastigmine, donepezil, memantine, and aducanumab, if the subject has AD.

### EXAMPLES

The following examples are provided to illustrate certain particular features and/or embodiments. These examples should not be construed to limit the disclosure to the particular features or embodiments described.

### Example 1

### Materials and Methods

Source of tissue samples. Islets from human non-diabetic (n=13) and T2DM (n=9) postmortem pancreata were provided by the NIDDK-funded integrated Islet Distribution Program (IIDP) at City of Hope, NIH Grant #2UC4DK098085 (iidp.coh.org/). Human postmortem control (n=20), AD (n=20) and Huntington Disease (HD, n=8) samples of middle temporal gyrus (MTG) and middle frontal gyrus (MFG) were obtained from the Division of Neuropathology, Department of Pathology, Johns Hopkins University School of Medicine, whose diagnoses were confirmed by autopsy and from which data have already been published (Airavaara et al., J. Biol. Chem. 286:45093-45102, 2011). Frozen human CP sections and blood samples (non-diabetic; n=5, T1DM; n=6, T2DM; n=4) were from the Human Brain Collection Core, NIMH (Hoffman et al., Sci. Data 6:180, 2019) (Table 1). Mouse pancreas, islets, CP and cortex were dissected as we previously described (Mazucanti et al., JCI Insight, 4:e131682, 2019).

TABLE 1
Clinical data of human post-mortem choroid plexus samples

| Age | Sex | Race | PMI | Ph | RIN | Smoker | BMI | Axis III | Cause of Death |
|---|---|---|---|---|---|---|---|---|---|
| 35.9 | M | African | 49.5 | 5.88 | 4.2 | No | 19.1 | Diabetes Mellitus, | THREE VESSEL |
| | | American | | | | | | Type I, (IDDM) | OCCLUSIVE |
| | | | | | | | | | CORONARY |
| | | | | | | | | | ATHEROSCLEROSIS |
| 56.3 | M | African | 31.5 | 6.09 | NA | Unknown | 31.3 | Diabetes Mellitus, | PULMONARY |
| | | American | | | | | | Type I, (IDDM) | EMBOLISM DUE TO |
| | | | | | | | | | DEEP VENOUS |
| | | | | | | | | | THROMBOSIS |
| 61.2 | F | Caucasian | 24.5 | 5.99 | 5.9 | No | 31.4 | Diabetes Mellitus, | CORONARY |
| | | | | | | | | Type I, (IDDM) | ARTERIOSCLEROSIS |
| 48.5 | F | African | 23.5 | 6.25 | 5.9 | Unknown | 26.9 | Diabetes Mellitus, | SEVERE GENERALIZED |
| | | American | | | | | | Type I, (IDDM) | ATHEROSCLEROSIS |
| | | | | | | | | | DUE TO |
| | | | | | | | | | HYPERTENSION |
| 41.5 | M | Caucasian | 34.5 | 6.47 | 8.4 | No | 27.2 | Diabetes Mellitus, | ATHEROSCLEROTIC |
| | | | | | | | | Type I, (IDDM) | CARDIOVASCULAR |
| | | | | | | | | | DISEASE |
| 84.2 | M | African | 27.5 | 6.27 | NA | No | 41.2 | Diabetes Mellitus, | MULTIPLE BLUNT |
| | | American | | | | | | Type I, (IDDM) | IMPACT INJURIES |
| 41.1 | M | African | 55.5 | 6.73 | NA | No | 24.1 | Diabetes Mellitus, | HYPERTENSIVE |
| | | American | | | | | | Type II (Non- | CARDIOVASCULAR |
| | | | | | | | | IDDM) | DISEASE WITH |
| | | | | | | | | | BIVENTRICULAR |
| | | | | | | | | | DILATION |
| 41.2 | F | African | 32 | 6.62 | 8.9 | Yes | 27.4 | Diabetes Mellitus, | ASHCVD WITH |
| | | American | | | | | | Type II (Non- | CORONARY ARTERY |
| | | | | | | | | IDDM) | STENOSIS |
| 54.9 | M | African | 27 | 6.11 | 8.4 | No | 42.1 | Diabetes Mellitus, | PULMONARY EBLOLUS |
| | | American | | | | | | Type II (Non- | DUE TO DEEP VENOUS |
| | | | | | | | | IDDM) | THROMBOSIS |
| 56.5 | M | African | 24 | 6.51 | 8.9 | No | 40.3 | Diabetes Mellitus, | PULMONARY EBLOLUS |
| | | American | | | | | | Type II (Non- | DUE TO DEEP VENOUS |
| | | | | | | | | IDDM) | THROMBOSIS |
| 27.6 | F | African | 21 | 6.41 | 8.3 | No | 52 | Non-Diabetes | ARRHYTHMOGENIC |
| | | American | | | | | | | RIGHT VENTRICULAR |
| | | | | | | | | | DYSPLASIA |
| 45.3 | M | African | 21.5 | 6.73 | 8.1 | No | 68.9 | Non-Diabetes | HYPERTENSIVE |
| | | American | | | | | | | CARDIOVASCULAR |
| | | | | | | | | | DISEASE AND MORBID |
| | | | | | | | | | OBESITY |
| 52.6 | F | African | 26 | 6.38 | 9.1 | No | 36.9 | Non-Diabetes | ACUTE FIBRINOUS |
| | | American | | | | | | | PERICARDITIS WITH |
| | | | | | | | | | CARDIAC TAMPONADE |
| 66.7 | F | Caucasian | 29.5 | 6.48 | 7 | No | 40.3 | Non-Diabetes | RUPTURED THORACIC |
| | | | | | | | | | AORTIC ANEURYSM |
| 45.4 | F | African | 43 | 6.43 | 8.1 | No | 21.9 | Non-Diabetes | RUPTURED |
| | | American | | | | | | | DISSECTING THORACIC |
| | | | | | | | | | AORTIC ANEURYSM |

PMI: postmortem interval, RIN: RNA integrity number; BMI: body mass index

Insulin levels were measured in plasma of non-diabetic adults after overnight fast and 2 hours after continuous intravenous glucose (2 hr-JVG) administration (glucose clamp methodology as previously described (Mager et al., J. Pharmacol. Exp. Ther. 311:830-835, 2004) whereby circulating glucose was clamped at fasting levels+98 mg/dL glucose during glucose infusion). This was undertaken to confirm that the MS-based assay (see below) was capable of quantifying endogenous INS products in controlled settings and for comparison with measurements in the same samples by ELISA.

TABLE 2
Clinical data of post-mortem human islet samples

| | | | HBA1c | Origin/ | | Diabetes | Glucose | | Warm | Cold | Est. | Est. | Total |
|---|---|---|---|---|---|---|---|---|---|---|---|---|---|
| | | BMI | or | source of | History of | duration | therapy | Cause | ischemia | ischemia | purity | viability | culture |
| Age | Sex | (kg/m2) | glucose | islets | diabetes? | (years) | at death | of death | time (h) | time (h) | (%) | (%) | time(h) |
| 41 | M | 28.5 | 165.0 | IIDP | No | | | Head | 0.4 | 12.8 | 95 | 95 | 38 |
| | | | mg/dl | | | | | trauma | | | | | |
| 37 | F | 19.7 | 122.4 | IIDP | No | | | Anoxia | 0 | 6.2 | 95 | 95 | 82 |
| | | | mg/dl | | | | | | | | | | |
| 51 | M | 35.6 | 187.8 | IIDP | Yes | 0-5 | Oral med | Stroke | 0 | 9.9 | 90 | 95 | 95 |
| | | | mg/dl | | | | | | | | | | |
| | | | A1c = 7.1 | | | | | | | | | | |
| 35 | M | 31.5 | 100.6 | IIDP | No | | 1 | Head | 0 | 14.5 | 90 | 95 | 96 |
| | | | mg/dl | | | | | trauma | | | | | |
| 46 | F | 21.5 | 136.2 | IIDP | No | | | Stroke | 0 | 4.2 | 95 | 95 | 48 |
| | | | mg/dl | | | | | | | | | | |
| 37 | M | 22.9 | 96.80 | IIDP | No | | | Anoxia | 0 | 7.4 | 95 | 90 | 22 |
| | | | mg/dl | | | | | | | | | | |
| 39 | M | 31.9 | 125.0 | IIDP | No | | | Stroke | 0 | 12.0 | 80 | 93 | 96 |
| | | | mg/dl | | | | | | | | | | |
| 41 | F | 19.1 | 230.0 | IIDP | No | | | Head | 0 | 6.9 | 94 | 94 | 42 |
| | | | mg/dl | | | | | trauma | | | | | |
| 51 | F | 22.5 | 179.6 | IIDP | No | | | Stroke | 0 | 11.3 | 95 | 95 | 111 |
| | | | mg/dl | | | | | | | | | | |
| 46 | F | 35.9 | 262.4 | IIDP | Yes | 0-5 | Oral med | Stroke | 0 | 6.9 | 50 | 92 | 18 |
| | | | mg/dl | | | | | | | | | | |
| | | | A1c = 6.8 | | | | | | | | | | |
| 52 | F | 42.8 | 237.4 | IIDP | Yes | 0-5 | Oral med | Stroke | 0 | 6.9 | 90 | 95 | 68 |
| | | | mg/dl | | | | | | | | | | |
| | | | A1c = 6.6 | | | | | | | | | | |
| 59 | M | 27.7 | 199.8 | IIDP | Yes | 6-10 | Oral med | Stroke | 0 | 6.1 | 85 | 95 | 59 |
| | | | mg/dl | | | | | | | | | | |
| | | | A1c = 6.5 | | | | | | | | | | |
| 28 | M | 29.2 | 232.0 | IIDP | No | | | Head | 0 | 3.5 | 85 | 98 | 66 |
| | | | mg/dl | | | | | trauma | | | | | |
| 45 | M | 36.4 | 174.0 | IIDP | No | | | Stroke | 0 | 9 | 90 | 93 | 24 |
| | | | mg/dl | | | | | | | | | | |
| 61 | M | 27.0 | 146.4 | IIDP | No | | | Head | 0 | ND | 80 | 98 | 70 |
| | | | mg/dl | | | | | trauma | | | | | |
| 27 | M | 30 | 108.00 | IIDP | No | | | Head | 0.53 | 4.3 | 95 | 99 | 43 |
| | | | mg/dl | | | | | trauma | | | | | |
| 16 | M | 31.5 | 209.2 | IIDP | No | | | Anoxia | 0 | 9.4 | 70 | 95 | 21 |
| | | | mg/dl | | | | | | | | | | |
| 26 | M | 28.7 | 142.0 | IIDP | Yes | 0-5 | Oral med | Head | 0 | 7.3 | 90 | 95 | 55 |
| | | | mg/dl | | | | | trauma | | | | | |
| | | | A1c = 6.5 | | | | | | | | | | |
| 37 | F | 38.1 | 253.8 | IIDP | Yes | 0-5 | Oral med | Stroke | 0 | 4.7 | 85 | 94 | 22 |
| | | | mg/dl | | | | | | | | | | |
| | | | A1c = 8.2 | | | | | | | | | | |
| 45 | M | 27.2 | 246.0 | IIDP | Yes | 0-5 | Oral med | Stroke | 0 | 11.4 | 85 | 95 | 101 |
| | | | mg/dl | | | | | | | | | | |
| | | | A1c = 6.5 | | | | | | | | | | |
| 54 | M | 24.5 | 122.4 | IIDP | Yes | 6 | Oral med | Stroke | 0 | 10.1 | 80 | 92 | 18 |
| | | | mg/dl | | | | | | | | | | |
| | | | A1c = 4.8 | | | | | | | | | | |
| 45 | M | 36 | 258.8 | IIDP | Yes | ND | Insulin | Stroke | 0 | 5.9 | 73 | 98 | 20 |
| | | | mg/dl | | | | 13.5 U | | | | | | |
| | | | A1c = 6.6 | | | | | | | | | | |
| 46 | F | 33.2 | 164.2 | IIDP | Yes | >10 years | Insulin | Stroke | 0 | 5.8 | 95 | 95 | 30 |
| | | | mg/dl | | | | 20 U | | | | | | |
| | | | A1c = 10.7 | | | | | | | | | | |
| 54 | M | 33.7 | 293.8 | IIDP | Yes | 0-5 | Insulin 8 | Stroke | 0 | 4.5 | 95 | 94 | 88 |
| | | | mg/dl | | | | U | | | | | | |
| | | | A1c = 8.0 | | | | | | | | | | |

Bioinformatics and Sanger sequencing of insulinoma cDNA clones. Sequencher 5.4.6 software (Gene Codes Corporation, Ann Arbor, MI, USA) was used to assemble pancreatic islet and insulinoma EST sequences (www.ncbi.nlm.nih.gov/dbEST/) that are homologous to human INS gene. National Center for Biotechnology Information (NCBI) BLAST (blast.ncbi.nlm.nih.gov/Blast.cgi) was used to search INSU1 uORF specific sequence against dbESTs of primate and other species. SignalP 4.1 Server (cbs.dtu.dk/services/SignalP/) (Petersen et al., Nat. Methods 8:785-786, 2011) and SecretomeP 2.0 Server (cbs.dtu.dk/services/SecretomeP/) (Bendtsen et al., Protein Eng. Des Sel 17:349-356, 2004) of ExPASy portal were used to predict signal peptide and non-signal peptide triggered protein secretion of INS isoforms, respectively. Human Splicing Finder (www.umd.be/HSF/HSF.shtml) was used to predict potential INS exon-3 intra-exonal splicing events (Desmet et al., Nucleic Acids Res. 37:e67, 2009). GlobPlot 2 (globplot.embl.de/) (Linding et al., Nucleic Acids Res. 31:3701-3708, 2003) was used to predict intrinsic protein disorder domain and globularity of INSU1 isoforms. Primate evolutionary nonsynonymous (Ka) and synonymous (Ks) substitution rates of INSU1 specific sequence was calculated (kakscalculator.herokuapp.com/) by Jukes-Cantor and Kimuras two parameter methods (Zhang et al., Genomics Proteomics Bioinformatics 4:259-263, 2006). The LASAGNA 2.0 (biogrid-lasagna.engr.uconn.edu/lasagna_search/) algorithm (Lee et al., Biotechniques 54:141-153, 2013) was used to search for transcription factor binding sites with the 1 kb 5′-flanking sequences of human INSU1 and mouse Ins2-V4. SRAMP (a sequence-based N6-methyladenosine m6A modification site predictor) was used to predict potential mammalian N6-methyladenosine m6A sites with RNA secondary structures of 5′UTR of INSU1 (Maurer-Stroh et al., J. Mol. Biol. 317:541-547, 2002). The cellular localization of INSU1 was predicted by SPOCTOPUS (Viklund et al., Bioinformatics 24:2928-2929, 2008). The extracellular glycosylation sites were predicted by NetGlycate (cbs.dtu.dk/services/NetGlycate/) (Johansen et al., Glycobiology 16:844-853, 2006) and NetOGlyc (cbs.dtu.dk/services/NetOGlyc/) (Steentioft et al., EMBO J. 32:1478-1488, 2013). Trypsin cleavage sites and efficiency (Siepen et al., J. Proteome Res. 6:399-408, 2007) for INSU1 were predicted by MC:pre (king.smith.man.ac.uk/mcpred/) and ProteinProspector (prospector.ucsf.edu). IMAGE cDNA clones were ordered from Source Bioscience (sourcebioscience.com/) and the inserts were sequenced by Sanger sequencing service of Eurofins Genomics (Louisville, KY, USA).

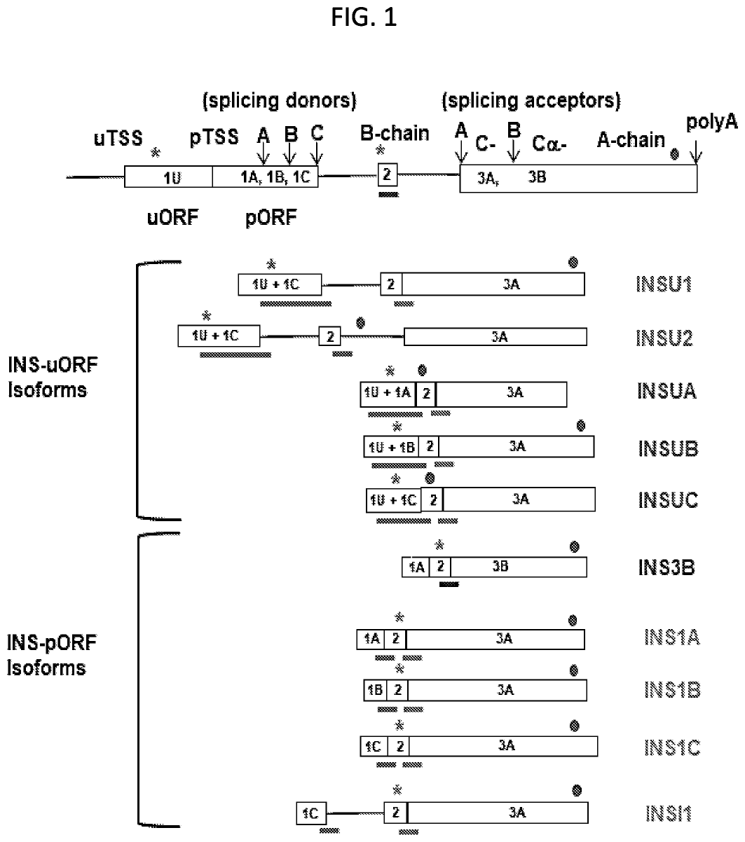

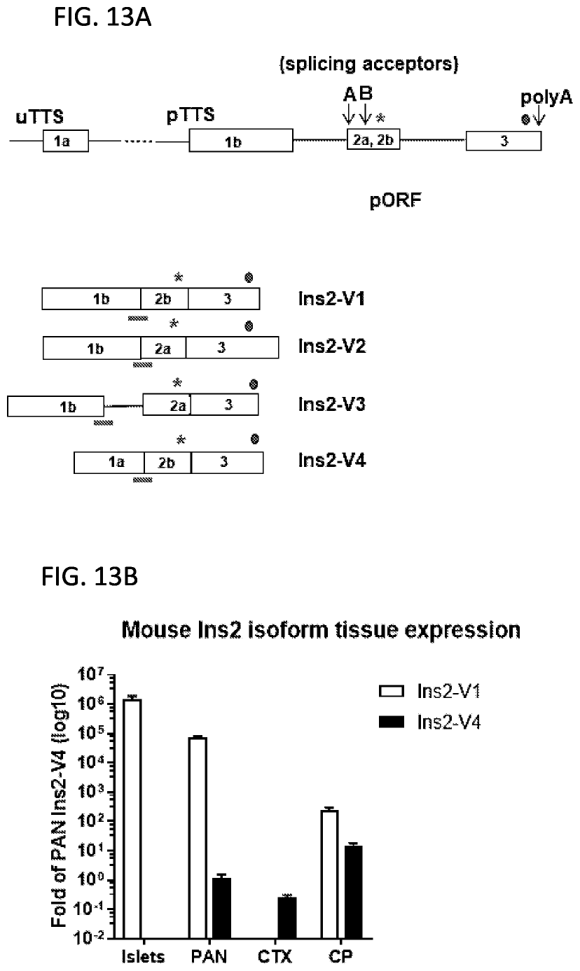

RNA isolation, cDNA synthesis and RT-qPCR. Total RNA was extracted from human islets, MTG, MFG, and CP, as well as mouse pancreas, islets, CP, and cortex using the Trizol (Thermo Fisher Scientific, Waltham, MA) protocol. Single strand cDNA was synthesized from total RNA using qScript™ XLT cDNA SuperMix (QuantaBio, Beverly, MA). For quantitative real-time PCR assessments of insulin isoform mRNAs, isoform-specific primers, and minor groove binding (MGB) FAM-labeled TaqMan probes were designed using Primer Express Software (Table 2). Splicing junction specific Taqman probes were designed for pORF isoforms (INS1A, 1B, 1C, I1, and 3B) and the uORF specific forward primer overlapping the INSU isoform translation initiation methionine and the TaqMan probes at the splicing junctions of exon-1UA/exon-2 (INSUA), exon-1UB/exon-2 (INSUB), exon-1UC/exon-2 (INSUC), and exon-1UC/intron-1 (INSU1) (FIG. 1) to measure expression of the uORF in human tissues. Since the exon-intron junction size of INSU2 is more than the amplicon limit for TaqMan assay, INSU2 transcription levels were measured by averaged values of INSU1 and EX2-I2 probes (FIG. 1). Splicing junction specific Taqman probes were also designed for mouse Ins2 isoforms (FIG. 13A: Ins2-V1, -V2, -V3 and -V4). Commercial FAM-labeled TaqMan probes of INS exon-2 and -3 junction (EX2-3A, Hs00355773_ml), IGF1 (Hs01547656_ml), GAD2 (Hs00609534_ml), PTPRN (Hs00160947_ml), SLC30A8 (Hs00545183_ml), ICA1 (Hs01119158_ml), MAFA (Hs01651425_s1), NEUROD1 (Hs01922995_s1), ISL1 (Hs00158126_ml), NKX6-1 (Hs00232355_ml), PDX1 (Hs00236830_ml), PAX4 (Hs00173014_ml), NEUROG3 (Hs01875204_s1), and endogenous control GAPDH (VIC-labeled, Cat #4326317E) were from Thermo Fisher Scientific. The duplex fluorescent TaqMan assay was performed in replicates (StepOnePlus™ real-time PCR system) and the relative fold change was calculated using the formula: 2\hat{ }(−ΔΔCt) (Liu et al., J. Neurochem. 128:173-185, 2014). Droplet Digital PCR (ddPCR) absolute values were derived from Poisson distribution of positive and negative droplets (QX200 ddPCR System (Bio-Rad, Philadelphia, PA) that were normalized with endogenous control β2 microglobulin (B2M Vic-labeled, Cat #4326319E).

TABLE 3
Splicing junction specific TaqMan probe and primer sequences of
human INS and mouse Ins2 isoforms

| Isoform | TaqMan probe | Forward primer | Reverse primer |
|---|---|---|---|
| EX2 | TGAACCAACACCTGTG | CCCAGCCGCAGCCTTT | AGAGAGCTTCCACCAGG |
| | CG (SEQ ID NO: 9) | (SEQ ID NO: 10) | TGTGA (SEQ ID NO: 11) |
| EX2-3A* | Hs00355773_m1 | Exon-2 (junction | Exon3A (junction 126 |
| | | 126 bp amplicon) | bp amplicon) |
| EX2-12 | ACGAGGCTTCTTCTACA | TCACACCTGGTGGAAG | GGCAGCAATGGGCAGTT |
| | CAC (SEQ ID NO: 12) | CTCTCT (SEQ ID NO: 13) | G (SEQ ID NO: 14) |
| INS1A | CCATCAAGCAGATCAC | GGACAGGCTGCATCAG | ACAGGGCCATGGCAGAA |
| | TGT (SEQ ID NO: 15) | AAGAG (SEQ ID NO: 16) | G (SEQ ID NO: 17) |
| INS1B | TTTGCGTCAGATCACT | CCATCAAGCAGGTCTG | GGGCCATGGCAGAAGG |
| | (SEQ ID NO: 18) | TTCCA (SEQ ID NO: 19) | A (SEQ ID NO: 20) |
| INS1C | ACCCCAGATCACTGTC | CCATCAAGCAGGTCTG | GCAGGAGGCGCATCCA |
| | (SEQ ID NO: 21) | TTCCA (SEQ ID NO: 22) | (SEQ ID NO: 23) |
| INSI1 | CCATCAAGCAGGTCTG | GGACAGGCTGCATCAG | AGCCCACCTGACGCAAA |
| | (SEQ ID NO: 24) | AAGAG (SEQ ID NO: 25) | G (SEQ ID NO: 26) |
| INSUA | CCATCAAGCAGATCAC | GGGAGATGGGCTCTGA | ACAGGGCCATGGCAGAA |
| | TGT (SEQ ID NO: 27) | GACTATAA (SEQ ID NO: | G (SEQ ID NO: 29) |
| | | 28) | |
| INSUB | TTTGCGTCAGATCACT | GGGAGATGGGCTCTGA | GGGCCATGGCAGAAGG |
| | (SEQ ID NO: 30) | GACTATAA (SEQ ID NO: | A (SEQ ID NO: 32) |
| | | 31) | |
| INSUC | ACCCCAGATCACTGTC | GGGAGATGGGCTCTGA | GCAGGAGGCGCATCCA |
| | (SEQ ID NO: 33) | GACTATAA (SEQ ID NO: | (SEQ ID NO: 35) |
| | | 34) | |
| INSU1 | CCATCAAGCAGGTCTG | GGGAGATGGGCTCTGA | AGCCCACCTGACGCAAA |
| | (SEQ ID NO: 36) | GACTATAA (SEQ ID NO: | G (SEQ ID NO: 38) |
| | | 37) | |
| INS3B | CAGGCTGCCCTGCAG | TAGAGGGAGCAGATGC | AGGCTTCTTCTACACAC |
| | (SEQ ID NO: 39) | TGGTACA (SEQ ID NO: | CCAAGA (SEQ ID NO: |
| | | 40) | 41) |
| Ins2-V1 | CAAGCAGGAAGGTTAT | CCGCTACAATCAAAAA | CATCCACAGGGCCATGT |
| | TGT (SEQ ID NO: 42) | CCATCA (SEQ ID NO: 43) | TG (SEQ ID NO: 44) |
| Ins2-V2 | CAAGCAGGAAGGTACT | CCGCTACAATCAAAAA | GAGCCAGGCCCACTGAG |
| | C (SEQ ID NO: 45) | CCATCA (SEQ ID NO: 46) | A (SEQ ID NO: 47) |
| Ins2-V3 | ATCCGCTACAATCAA | CCAGTAACCACCAGCC | AGATAGGCTTCCTGCTT |
| | (SEQ ID NO: 48) | CTAAGT (SEQ ID NO: 49) | GCTGATG (SEQ ID |
| | | | NO: 50) |
| Ins2-V4 | CTGGAGGTTATTGTTTC | GCTCCTACGCTGAAATT | AAGGTGCTGCTTGACAA |
| | AA (SEQ ID NO: 51) | CCAA (SEQ ID NO: 52) | AAGC (SEQ ID NO: 53) |

* ThermoFisher Scientific, proprietary sequences

RNAscope fluorescent in situ hybridization (ISH) in human islets and brain. RNAscope ISH probes were custom designed for INSU isoform (11 ZZ pairs targeting 2-272 of uTSS and 273-732 of pTSS nucleotide sequence of MT335691 in C1 or C2 channel). TTR (18 ZZ pairs targeting 2-917 nucleotides of NM_000371.3 in C1 channel), IAPP (16 ZZ pairs targeting 424-1947 nucleotides of NM_000415.2 in C3 channel), and RBFOX3 (NEUN: 20 ZZ pairs target region 720-2217 of NM_001082575.2 in C3 channel) were from cataloged probes from Advanced Cell Diagnostics Inc. (ACD, Hayward, CA). Isolated islets were individually handpicked and embedded with Shandon M-1 Embedding Matrix (Thermo Fisher Scientific), frozen in ethanol/dry ice bath, and stored at −80° C. before sectioning. Human postmortem MTG was used for duplex fluorescent ISH with INSU and NEUN probes. Sections (15 μm) of CP and MTG were obtained in a cryostat microtome and then fixed with 10% neutral buffered formalin (NBF) immediately before hybridization and staining. Pretreatment of brain sections, probe reactions, and labeling was performed according to RNAscope Multiplex Fluorescent Detection Kit v2 protocol, and as previously described (Liu et al., Acta Pharmacol. Sin. 40:387-397, 2019). The negative control probe was a universal control probe targeting bacterial Dapb gene (GenBank accession number: EF191515) from the Bacillus subtilis strain. The positive controls were human probes (Cat No. 320861) for RNAscope Multiplex Fluorescent Assay POLR2A (C1) and PPIB (C2), UBC (C3). Images were acquired using a Zeiss LSM 880 confocal microscope.

Enzyme-linked immunosorbent assay (ELISA) for plasma and postmortem frozen blood. Plasma samples were assayed for insulin (Mercodia, Winston Salem, NC Cat #10-1113-01) and C-peptide (Mercodia, Cat #10-1136-01) by ELISA (Chia et al., Am. J. Physiol. Endocrinol. Metab. 313:E359-E366, 2017). Proteins were precipitated from postmortem frozen blood samples by orderly mixing 1 ml of ice-cold acetone, 0.125 ml trichloroacetic acid (6.1 N), and 0.125 ml of thawed frozen blood and vortex vigorously for 1.5 min. After −20° C. overnight precipitation, the pellets were collected by centrifugation at 13,000×g for 15 min at 4° C. and washed 5 times by vigorous vortex and centrifugation at 8,000×g for 5 min with 1 ml of acetone and air dry completely before dissolving in 0.5 ml phosphate-buffered saline (PBS) for C-peptide ELISA (Palazzoli et al., J. Pharm. Biomed. Anal. 150:25-32, 2018).

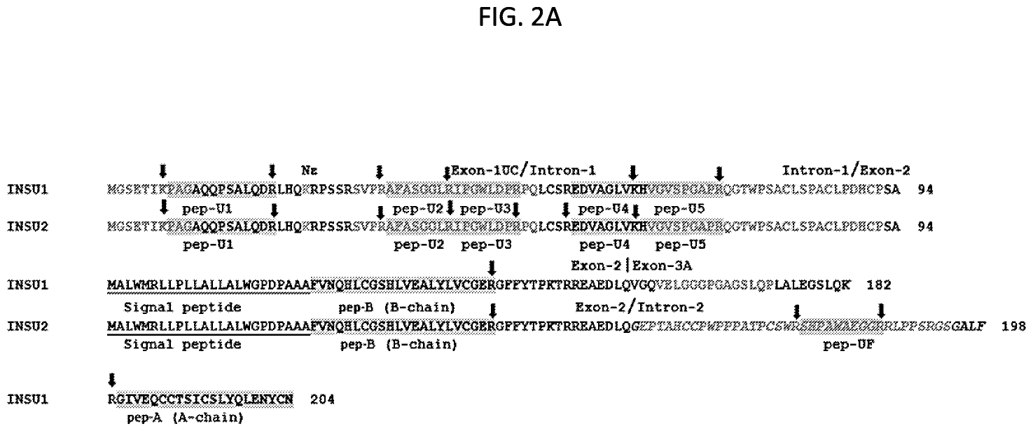

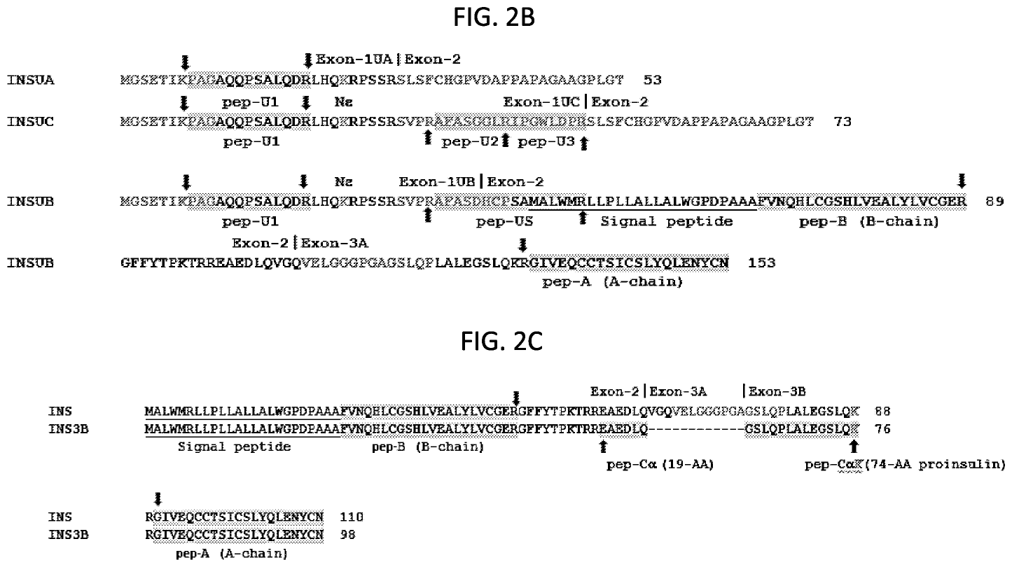

Development of mass-based Selected Reaction Monitoring (SRM-MS) assay. Briefly, the method includes: 1) the selection of potential tryptic peptides based on the functional and alternative splicing sites, i.e. pep-U1, -U2, and -U3 peptides are encoded by the exon-1U, and the pep-U4 and pep-U5 peptides are non-canonically encoded by intron-1, and pep-UF (frameshift) encoded by intron-2 (FIG. 1 and FIGS. 2A-2B). Pep-US is encoded by spliced exon-1UB and exon-2 (FIG. 2B and FIGS. 3A-3G), the tryptic pep-U3 is measured and validated by SRM-MS with stable isotope labeling (FIG. 3C) because the arginine digestion site (0) is flanked by prolines at −1 (promoting) and +1 (inhibiting) sites (Pan et al., Anal. Bioanal. Chem. 406:6247-6256, 2014). The pep-B (B-chain) is encoded by exon-2 and located after the signal peptide, while the pep-A (A-chain) is encoded by exon-3 (FIGS. 3H and 3I) and is the same as the complete A-chain peptide. The 19-AA pep-CD (Cα) and non-processed pep-CαK ((FIG. 2C, CαK, as a surrogate for the 74-AA proinsulin) are derived from INS3B3 that is alternatively spliced exon-2 and exon-3B3 (FIGS. 3J and 3K) the selected peptides were synthesized as isotope-labeled and unlabeled analogues (Table 3) by Genemed Synthesis Inc. (San Antonio, TX), and after reconstitution each peptide concentration was determined by amino acid analysis (New England Peptide, Gardner, MA); 3) the optimal charge state, declustering potential (DP), collision energy (CE), collision cell exit potential (CXP) were selected as detailed elsewhere (Zhu et al., Proteomics 17, doi.org/10.1002/pmic.201600339, 2017). Three to six interference-free precursors and fragment ion masses (transitions) for a given peptide were constituted in the final multiple SRM assay. For further enhancement of SRM sensitivity, the mass spectrometer collected subsets of peaks based on the target analyte retention times (RT) on the column; 4) analytical validations for SRM assay performance.

TABLE 4
Unlabeled and isotope-labeled (marked by asterisk) tryptic
peptide sequences and their molecular weights (MW; dal,
Dalton) for SRM-MS assay.

| | | | | SEQ |
|---|---|---|---|---|
| Peptide | | MW | | ID |
| name | Peptide sequences | (dal) | AA | NO: |
| pep-B | H₂N-FVNQHLC[CAM]GSHLVEALYLVC[CAM]GER-OH | 2601.27 | 22 | 54 |
| pep-A | H₂N-GIVEQC[CAM]C[CAM]TSIC[CAM]SLYQLENYC | 2611.10 | 21 | 55 |
| | [CAM]N-OH | | | |
| pep-Cα | H₂N-EAEDLQGSLQPLALEGSLQ-OH | 1998.00 | 19 | 56 |
| pep-CαK | H₂N-EAEDLQGSLQPLALEGSLQK-OH | 2126.10 | 20 | 57 |
| pep-U1 | H2N-PAGAQQPSALQDR-OH | 1338.67 | 13 | 58 |
| pep-U2 | H2N-AFASGGLR-OH | 778.42 | 8 | 59 |
| pep-U3 | H2N-IPGWLDPR-OH | 953.52 | 8 | 60 |
| pep-U4 | H₂N-EDVAGLVK-OH | 830.46 | 8 | 61 |
| pep-U5 | H₂N-HVGVSPGAPR-OH | 976.53 | 10 | 62 |
| pep-US | H2N-AFASDHC[CAM]PSAMALWMR-OH | 1850.81 | 16 | 63 |
| pep-UF | H₂N-SHPAWAEGGR-OH | 1067.50 | 10 | 64 |
| pep-B* | H₂N-FV\hat{ }NQHLC[CAM]GSHLVEALYLVC[CAM]GER-OH | 2609.23 | 22 | 54 |
| pep-A* | H₂N-GIV\hat{ }EQC[CAM]C[CAM]TSIC[CAM]SLYQLENYC | 2619.01 | 21 | 55 |
| | [CAM]N-OH | | | |
| pep-Cα* | H2N-EA\hat{ }EDLQGSLOPLA\hat{ }LEGSLQ-OH | 2004.00 | 19 | 56 |
| pep- | H₂N-EAEDLQGSLQPLALEGSLQK\hat{ }-OH | 2134.10 | 20 | 57 |
| CαK* | | | | |
| pep-U1* | H2N-PAGAQQPSALQDRA-OH | 1348.00 | 13 | 58 |
| pep-U2* | H₂N-AFASGGLR\hat{ }-OH | 788.42 | 8 | 59 |
| pep-U3* | H2N-IPGWLDPRA-OH | 963.00 | 8 | 60 |
| pep-U4* | H₂N-EDVAGKVK\hat{ }-OH | 838.46 | 8 | 61 |
| pep-U5* | H₂N-HVGVSPGAPRA-OH | 986.53 | 10 | 62 |
| pep-US* | H2N-A\hat{ }FA\hat{ }SDHC[CAM]PSA\hat{ }MA\hat{ }LWMR-OH | 1862.00 | 16 | 63 |
| pep-UF* | SHPA\hat{ }WA\hat{ }EGGR-OH | 1073.14 | 10 | 64 |

CAM = carbamidomethylation of cysteine residues
\hat{ }= isotope labeled amino acid residues. Valine and alanine residues were labeled with deuterium; lysine and arginine were labeled with carbon-13 and nitrogen-15

In greater detail, under a stereo microscope, approximately 200 intact islets from individual donors were handpicked into a polypropylene tube containing 1 ml of ice-cold PBS. After washing twice with 1 ml PBS containing 1× protease inhibitor cocktails, the islets were resuspended in 100 μl of 0.1% RapiGest (w/v) (Waters Corp., Milford, MA) containing 100 mM Tris-HCl (pH 8.0) and 100 nM DTT. For choroid plexus postmortem frozen sections 100 μl of 0.1% RapiGest was added on each slide, and then tissue was scraped off into a clean 1.5 ml of tube. Then, islet and choroid plexus in 0.1% RapiGest were sonicated 3×3 seconds on/30 seconds off on ice. After centrifugation (16,000 g, 20 min at 4° C.), supernatants were collected and protein concentration was determined by BCA assay (Cat #: 23225, Thermo Fisher Scientific, Waltham, MA), and stored at −80° C. until further analysis. Due to low amounts of islet lysate, the islet protein concentration was not determined, and thus relative quantification was used for quantification, e.g., the ratio of CαK/Cα.

Brain MTG and MFG tissues were homogenized in 500 μl of 0.1% RapiGest with 100 mM Tris-HCl (pH 8.0) and 100 nM DTT. The lysates were further solubilized by incubation for 1 hour at 4° C. with continuous rotation. After centrifugation (16,000 g, 20 min at 4° C.), the supernatant was collected, and the protein concentration was determined by protein BCA assay. All islet lysate and 150 μg of brain CP, MTG and MFG lysates were used for the digestion procedure.

Fresh-frozen human plasma was thawed on the day of analysis. After centrifugation (16,000 g, 15 min at 4° C.), cleared fractions were transferred with a loading tip into a fresh 1.5 mL polypropylene tube, discarding insoluble aggregates and the upper layer of floating lipids. This procedure was enough to eliminate the confounding influence of lipids on downstream protein separation procedures. Then 5 μl of delipidated plasma was mixed with 95 μl of 0.1% RapiGest.

Tryptic digestion was performed with an automated robotic procedure aimed at minimizing sample handling variability in a flow for SRM analysis (Zhu et al., Proteomics 17, doi.org/10.1002/pmic.201600339, 2017). Briefly, sample lysate (100 μl) in 0.1% RapiGest were transferred into the reaction plate, incubated 1 hour at 55° C. for denaturation and reduction, followed by 30 min alkylation with a fresh made 0.1 M solution of iodoacetamide (Sigma-Aldrich) to a final concentration of 50 mM at room temperature in the dark. After alkylation, trypsin/LysC mix (Promega, Madison, WI) was added at an enzyme-to-substrate ratio of 1:50. Digestion was carried out for 18 hours at 37° C. and terminated with 10% MS-grade trifluoroacetic acid (Fisher Scientific, Hampton, NH) to a final concentration of 1%. Acidified tryptic digests were cleaned up with 96-well SPE plate (Phenomenex, Torrance, CA) according to manufacturer's instruction. A 96-well plate vacuum manifold (Waters Corp., Milford, MA) was used for all desalting procedures to provide uniform peptide wash, retention, and elution. The elution reagents were evaporated to dryness and stored at −80° C. until SRM analysis. All internal standard peptides of the novel INS uORF isoforms (INSU1 and INSU2) were post-spiked into tryptic digests.

A Shimadzu LC-HPLC equipped with LC-20ADXR pumps (Shimadzu Corp., Columbia, MD) was used for solvent and sample delivery and a 2.1 mm×100 mm, 130 Å pore size, 3.5 m particle size C18 column (Waters Corp.) was used for the peptide separations using the following linear gradient: 0 min 5% B; 10 min 36% B; 12 min 90% B; 13.5 min 90% B; 14 min 5% B at a flow rate of 0.2 ml/min. The total run time was 18 min per sample. Triplicate injections of 10 μl of sample were carried out via the SIL-20AXR autosampler (Shimadzu Corp.). To eliminate possible carryover, the column was re-equilibrated at 50% B for 10 min and a blank run was performed prior to initiating the next sample injection. QTRAP 5500 mass spectrometer with electrospray ionization (ESI) source controlled by Analyst 1.6.3 software (AB Sciex, Framingham, MA) was used for all LC-MS/MS detection and analysis. Mass spectrometric analyses were performed in positive ion mode. ESI interface parameters were set as follows: capillary temperature 650° C. and a curtain gas setting of 30 psi. By using scheduled SRM, a total of 148 SRM transitions from 15 peptides were monitored during an individual sample analysis with Q1 and Q3 set by declustering potential (DE) 10 V and peptide-specific tuned collision energy (CE), entrance potential (EP) and collision cell exit (CXP) voltages for each transition.

Statistical data analysis. GraphPad Prism v8.3 software was used for statistical analysis and data are presented as means±SEM. The normalized expression values of INS isoforms TaqMan fold changes and ddPCR percentage of target/B2M positive droplets, ELISA, and SRM quantitative signals were analyzed with one-way ANOVA, two-way ANOVA (repeated measures, subject matching with Sidak correction), unpaired student t-test, and P<0.05 was considered significant for comparisons of mRNA and protein levels.

### Example 2

### Identification of Upstream Open Reading Frames of INS (INSU) Isoforms

Exon-1 and -3 of INS are alternatively spliced, while exon-2 is a constitutive exon within which a translation initiation site resides (FIG. 1, green asterisk). The classical insulin is transcribed from exon-1A (INS1A), -1B (INS1B), and -1C (INS1C) including one with intron-1 retention (INSI1) (FIG. 1, green lettering). Assembly of 3,544 human insulinoma and islet expressed sequence tags (ESTs) identified that many of them retain intron-1 and -2. In addition, several cDNA clones contain alternative transcription start site (uTSS) upstream of the conventional INS 5′-cap site. Furthermore, the uTSS nucleotide sequence contains a consensus human Kozak ribosomal binding site (TGGGAGATGGGC; SEQ ID NO: 65) for an alternative translation initiation 45 bp upstream of the 5′-cap site (FIG. 1, red asterisk). The uORF of INSU1 is in-frame with the primary open reading frame (pORF) with retention of intron-1 and could be potentially translated to a 204-AA polypeptide while the uORF of INSU2 (insulinoma BioSample: SAMN00164222) retains introns-1 and -2 producing potentially a frameshifted 198-AA polypeptide (FIG. 1, red lettering, FIG. 2A). The presence of INSU1 clones were validated by Sanger sequencing of IMAGE clones (www.imageconsortium.org/) of IMAGp998A2012483Q (BM85746) and IMAGp99800113413Q (BU782803) in both directions. The uORF containing exon-1UA, -1UB, and -1UC could be potentially spliced to exon-2, generating INSUA, INSUB, and INSUC encoding polypeptides of 53, 153 and 73 AA, respectively (FIG. 1, FIG. 2B). INSUA and INSUC contain premature stop codons (FIG. 1, red dot) while INSUB is in frame with the pORF (FIG. 1, FIG. 2B).

### Example 3

### Primate Evolution and Amyloidogenic Propensity of INSU Isoforms

INSU1 uORF (FIG. 2A) is 95.75% identical to that in chimpanzee (Pan troglodytes, NC_036890.1) whose lineage diverged from Homo sapiens approximately 6 MYA (Nei et al., Proc. Natl. Acad. Sci. USA 98:2497-2502, 2001). Based on Jukes-Cantor's 1-parameter (the rate of nucleotide substitution) and Kimura's 2-parameters (the rates of separate nucleotide transition and transversion) models the 94-AA uORFs of human and chimpanzee have 0.03 nonsynonymous and 0 synonymous values, indicating that INSU1 is a fast evolving protein coding isoform (Wang et al., Biol. Direct 6:13, 2011). The coding region of human full-length INSU1 (204 AA) is under purifying selection (selective removal of deleterious alleles) against chimpanzee with Ka/Ks (the ratio of the number of nonsynonymous substitutions per non-synonymous site Ka, in a given period of time, to the number of synonymous substitutions per synonymous site Ks, in the same period) substitution ratio of 0.538 (Zhang et al., Genomics Proteomics Bioinformatics 4:259-263, 2006). The INSU1 of other primate species, gorilla (7MYA, NC_018435.2), orangutan (13 MYA, NC_036914), gibbon (17 MYA, NC_019819.1), rhesus monkey (25 MYA, NC_041767.1), baboon (25 MYA, NC_018165.2), and marmoset (33 MYA, NC_013906.1), are not in-frame with the pORFs (Nei et al., Proc. Natl. Acad. Sci. USA 98:2497-2502, 2001). No significant conservation of the INSU isoforms was found outside of the primate order. Therefore, the uORF isoforms and their promoter evolved in the Neogene period (23 to 2.6 MYA), and continued into the Quaternary period (2.58 to 0.012 MYA) and the current Anthropocene geological time scales.

The uORF of INSU1 lacks its predicted signal peptide cleavage site (SecretomeP prediction) and SPOCTOPUS algorithm (Viklund et al., Bioinformatics 24:2928-2929, 2008) predicted a single transmembrane domain (93-112 AA) that overlaps with the INS pORF signal peptide. The Lys-24 of INSU1 is predicted with a high score of 0.96 (Johansen et al., Glycobiology 16:844-853, 2006) to be NF-carboxymethyllysine (CML) that has potential to serve as a substrate for glycation and formation of advanced glycation end products (AGEs) (Fu et al., J. Biol. Chem. 271:9982-9986, 1996). GlobPlot 2.3 algorithm predicted that the 94-AA of INSU1 uORF contains three (marked by red AAs in FIG. 2A) intrinsically disordered protein regions (IDPRs) that are potentially amyloidogenic (Galzitskaya et al., PLoS Comput. Biol. 2:e177, 2006). The uORF of INSUA, INSUB, and INSUC peptides are predicted to contain IDPRs, all of which contain the Lys-24 CML glycation site (marked by blue K and NF in FIGS. 2A and 2B). The frame shift of INSU2 isoform replaces partial C-peptide and the entire A-chain with a 42-AA polypeptide—a 37-AA region of which is an IDPR (FIG. 2A, red and italic AA).

### Example 4

### Identification of Additional Insulin Isoform INS3B and Cα-Peptide

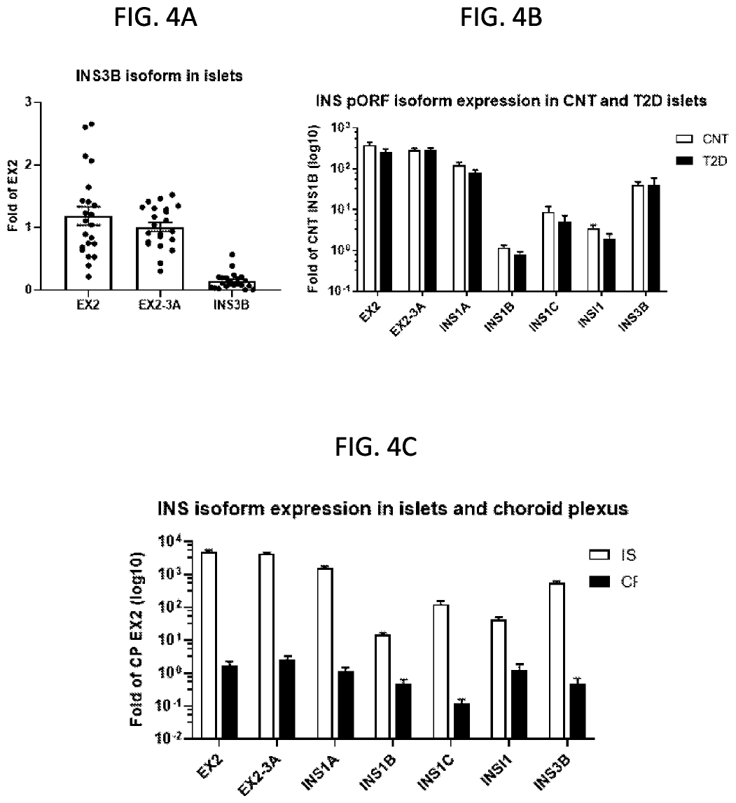

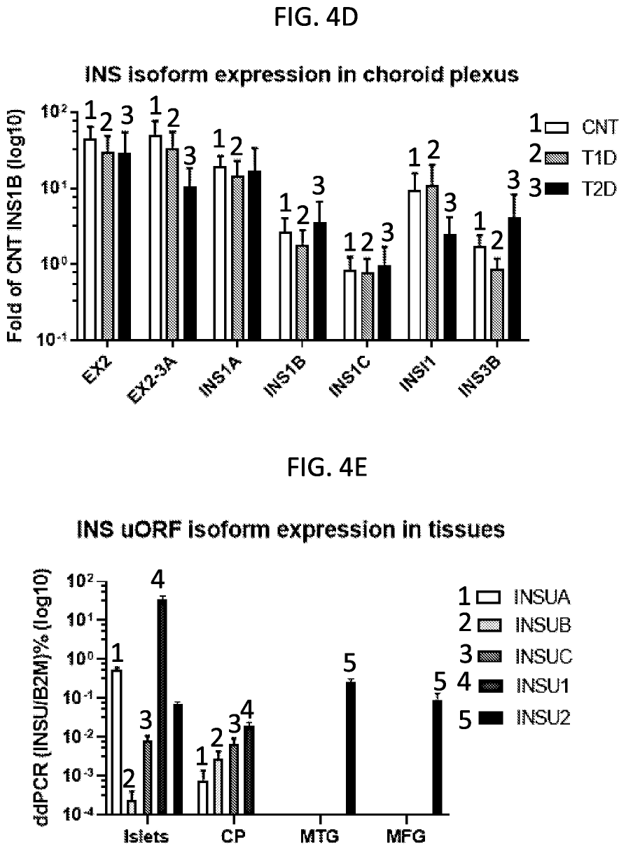

Using Human Splicing Finder (HSF) bioinformatic tool, it was determined that the third exon contains a potential intra-exonal splicing acceptor (>85% HSF matrix) site 36 bp downstream of the conventional exon-3A splicing acceptor site (FIG. 1). This was named the intra-exon-3 spliced isoform INS3B (FIG. 1, purple lettering) that translates to a 74-AA proinsulin within which resides a 19-AA C-peptide (Cα) instead of the classical 86-AA proinsulin with its C-peptide containing 31-AAs (FIG. 2C). The Cα-peptide (EAEDLQGSLQPLALEGSLQ; SEQ ID NO: 66) does not contain a (3-sheet (GQVEL; SEQ ID NO: 67) forming motif (Tsiolaki et al., Biopolymers 108, doi: 10.1002/bip.22882, 2017) that is present in the 31-AA C-peptide, and therefore does not have amyloidogenic properties. Additionally, the proinsulin of INS3B is predicted to have a soluble globular structure without the IDPR site (VELGGGPGAGSLQP; SEQ ID NO: 68) that is found in 31-AA C-peptide (FIG. 2C). The splicing junction specific TaqMan probes of EX2-3A and INS3B (FIG. 1, green and purple bars, respectively) were used to determine the expression levels of classical INS (encoding 31-AA C-pep) and INS3B (encoding 19-AA Cα-pep) in islets where they made up 85.2±7.2% (n=22) and 14.6±2.7% (n=22), respectively, of the total INS expression (FIG. 4A) measured by using the EX2 TaqMan probe (FIG. 1, blue bar): exon-2 exists in all INS isoforms (FIG. 1).

### Example 5

### Tissue Expression of INS Isoforms and Related Molecules

pORF isoforms that had similar expression levels in both control and T2DM islets (FIG. 3B) were detected by RT-qPCR. All the INS pORF isoforms were expressed in both islets and CP, indicating that CP contains similar INS pre-mRNA splicing machinery to β-cells (FIG. 4C). The total INS (EX2 TaqMan probe) mRNA level was 2,827 (±1160) fold higher in islets than in CP. The highly abundant INS1A, INS1C, and INS3B mRNA levels were more than 1000-fold greater in islets than in CP while the lower expressed INS1B mRNA levels were 31-fold higher in islets. The intron-1 retention INSI1 isoform mRNA levels were 33-fold higher in islets (FIG. 4C). In addition, postmortem CP from T1DM and T2DM contained INS isoforms at similar levels (FIG. 4D); it was verified by ELISA in the stored blood samples of those who were receiving insulin therapy that C-peptide was absent in T1DM samples. Additionally, by ddPCR uORF spliced isoforms (INSUA, UB, UC and U1) were detected in both islets and CP but not in MFG or MTG. On the other hand, the non-spliced isoform INSU2 was not detected in CP only while the cortex expressed only INSU2 (FIG. 4E).

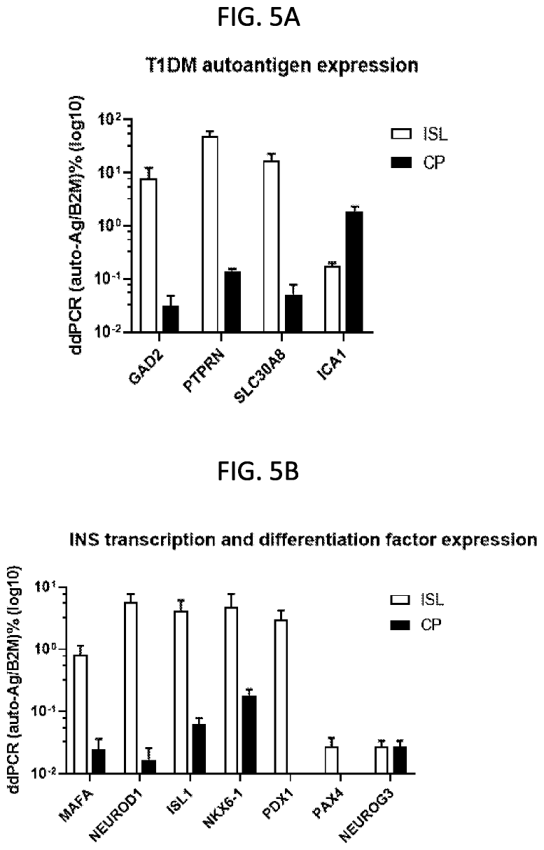

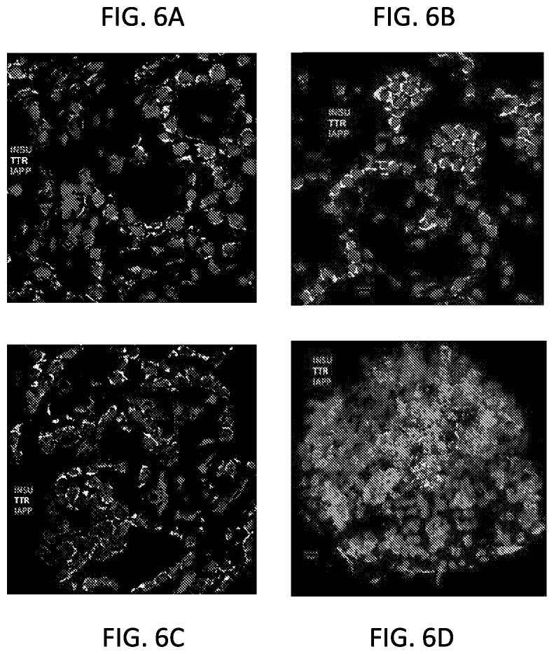

Expression of autoantigens and islet differentiation factors were compared between islets and CP using ddPCR. Except for ICA1 (islet cell autoantigen 1), expression of which was 10-fold higher in CP than in control islets, expression of other known autoantigens (GAD2, PTPRN and SLC30A8) was 253-, 346- and 339-fold higher in islets (FIG. 5A), respectively. Endocrine differentiation factor NEUROG3 (neurogenin-3) mRNA levels were similar, while expression levels of several other critical β-cell transcription factors (MAFA, NEUROD1, ISL1, NKX6-1) were 33-, 344-, 66- and 27-fold higher in islets (FIG. 5B), respectively. PDX1 (pancreatic and duodenal homeobox 1) and PAX4 (paired box gene 4) were absent from CP (FIG. 5B). Using triplex RNAscope ISH assay, it was determined that INS mRNA colocalized with TTR (transthyretin) in control (FIG. 6A), T2DM (FIG. 6B) and T1DM (FIG. 6C) CP samples, and as was previously reported in mice (Mazucanti et al., JCI Insight, 4:e131682, 2019), the IAPP (islet amyloid polypeptide) gene was not present in CP. This observation contrasts with pancreatic islets where INS colocalized with IAPP, as expected in β-cells, but did not co-localize with TTR (FIG. 6D); in islets transthyretin is present in α-cells, as previously reported (Su et al., FEBS Lett. 586:4215-4222, 2012).

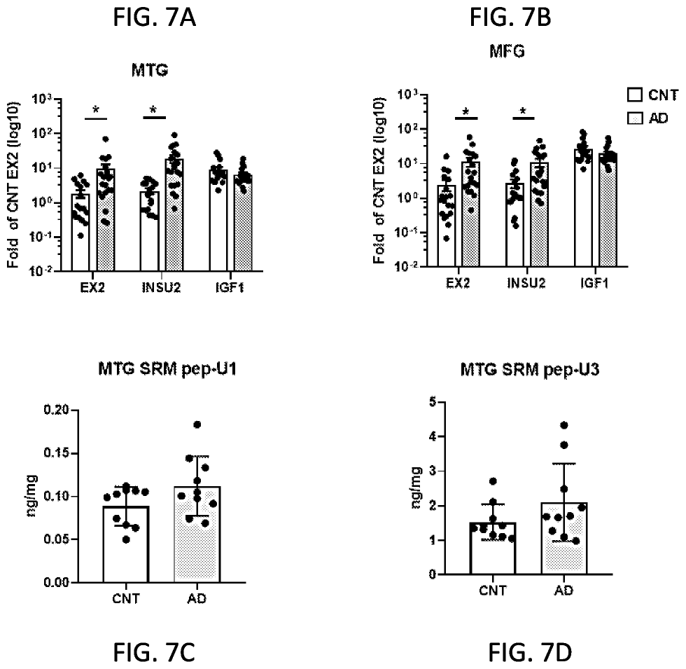

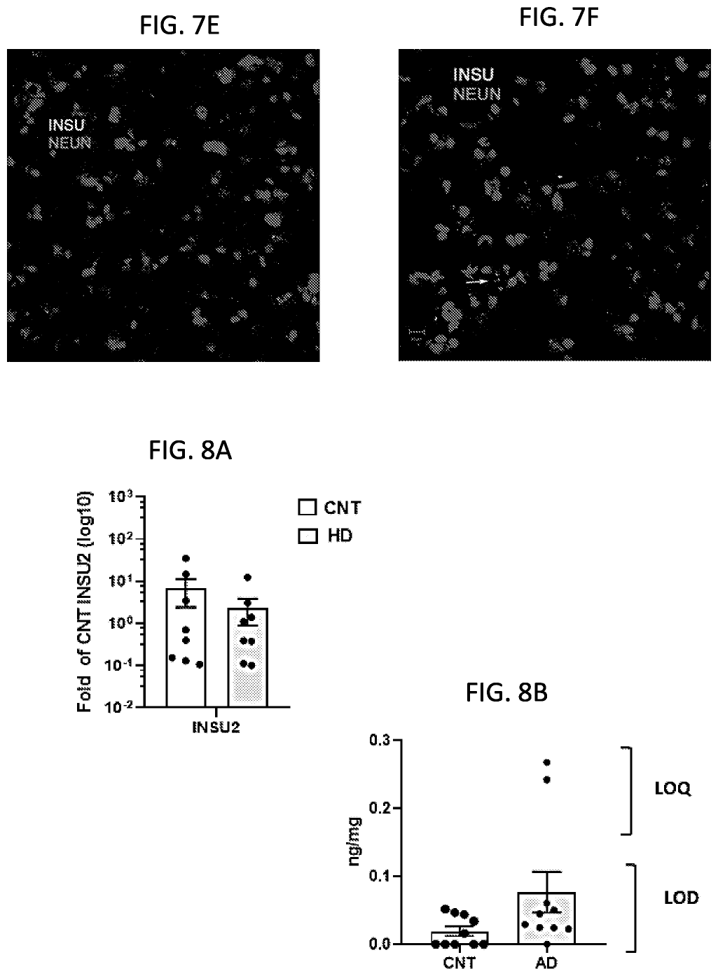

mRNA expression of the pORF spliced isoforms (INS1A, INS1B, INS1C, and INS3B) or the uORF spliced isoforms (INSUA, INSUB, INSUC and INSU1) was not detected in either control or AD MTG and MFG samples by RT-qPCR. However, in contrast, the non-spliced INSU2 isoform was found to be present in both control and AD MTG and MFG samples. The average mRNA level of INSU2 was more than 10-fold higher in AD MTG (p=0.005) and MFG (p=0.011, FIGS. 7A and 7B) while those of Huntington Disease (HD) MTG samples were similar to control (FIG. 8A). IGF1 (insulin-like growth factor 1) expression was slightly lower in the AD brains (FIGS. 7A and 7B). Evidence for higher levels of translational products of uORF isoforms (FIG. 3A-3G)—as measured by pep-U1 (p=0.095) and pep-U3 (p=0.166)—was found in AD MTG samples (FIG. 2A, 7C, 7D), though not reaching significance. SRM transitions of pep-UF (encoded by intron-2) in the two AD MTG samples were above the limit of quantification (LOQ) while the rest of the MTG samples were below LOQ (FIG. 8B). Using RNAscope INSU isoform probe (exon-1U, -2 and -3) and NEUN (RBFOX3) probe duplex ISH assay, it was found that INSU was barely detectable in control MTG samples but colocalized with neuronal marker NEUN in AD MTG samples (FIGS. 7E and 7F).

### Example 6

### SRM-MS Quantification of A- and B-Chains, Cα-Peptide and CαK-Peptide

The SRM-MS assay was quantitatively validated in pooled postmortem cerebrum samples (Table 5) and in pooled human plasma (Table 6).

TABLE 5
Quantitation validation of SRM-MS assay in pooled postmortem cerebrum

| | SRM | | | |
|---|---|---|---|---|
| Target | | ^{b} LOQ | CV | ^{c} Accuracy |
| peptide | ^{a} Linear Regression and R\hat{ }2 | (nM) | (%) | (mean ± SD, %) |
| Pep-B | log₂ Con(nM) = 7.035 + 0.946 * log₂Pep-B1 | 0.59 | 3.22 | 100.54 ± 9.12 |
| | (ratio, L/H); R\hat{ }2 = .998 | | | |
| Pep-A | log₂ Con (nM) = 9.685 + 1.065 * log₂Pep-A | 4.69 | 7.03 | 101.11 ± 6.40 |
| | (ratio, L/H); R\hat{ }2 = .998 | | | |
| Pep-Cα | log₂ Con(nM) = 7.51 + 1.04 * log₂Pep-Cα | 6.25 | 3.43 | 98.28 ± 8.76 |
| | (ratio, L/H)); R\hat{ }2 = .997 | | | |
| Pep-CαK | log₂ Con(nM) = 7.32 + 0.97 * log₂Pep-CαK | 0.26 | 3.56 | 100.28 ± 6.60 |
| | (ratio, L/H); R\hat{ }2 = .999 | | | |
| Pep-U1 | log₂ Con(nM) = 7.636 + 1.017 * log₂Pep-U1 | 0.38 | 2.04 | 100.23 ± 5.53 |
| | (ratio, L/H); R\hat{ }2 = 1 | | | |
| Pep-U3 | log₂ Con(nM) = 6.926 + 1.031* log₂Pep-U3 | 3.32 | 0.81 | 100.26 ± 7.43 |
| | (ratio, L/H); R\hat{ }2 = .999 | | | |
| Pep-U4 | log₂ Con(nM) = 10.655 + 1.02 * log₂Pep-U4 | 2.34 | 3.34 | 100.39 ± 10.08 |
| | (ratio, L/H); R\hat{ }2 = .998 | | | |
| Pep-UF | log₂ Con(nM) = 7.519 + 0.991 * log₂Pep-UF | 0.21 | 5.95 | 100.40 ± 8.57 |
| | (ratio, L/H); R\hat{ }2 = .999 | | | |
| Pep-US | log₂Con (nM) = 8.257 + .929 * log₂Pep-US | 7.81 | 2.87 | 100.30 ± 8.57 |
| | (ratio, L/H); R\hat{ }2 = .998 | | | |

^{a} Linearity was determined by linear regression between measured concentration and peak area ratio (L/H) versus theoretical concentration determined by Light-SIS peptides (unlabeled form). All output of MultiQuant were weighted by 1/χ². Sum of all transition peak area ratio (L/H) to get peak area ratio at the peptide level.
^{b} LOQ was determined from the standard curve, defined as the lowest limits of quantification calibrated with acceptable CV <20% and accuracy within 100 ± 20%.
^{c} Accuracy was estimated by back fitting data to the STD curve and average recovery from all quantified points in the plots. Data are mean ± SD (%). Calibration curve is based on internal standard (IS unlabeled form) concentration and peak area ratio (transition ion peak area/IS peak area) and prepared in a pooled plasma matrix which was prepared from studying plasma samples >=5 points.

TABLE 6
Quantitation validation of SRM-MS assay in pooled human plasma

| | SRM | | | |
|---|---|---|---|---|
| Target | | ^{b} LOQ | CV | ^{c} Accuracy |
| peptide | ^{a} Linear Regression and R2 | ng/ml | (%) | (%) |
| Pep-B | log₂Con(ng/ml) = 6.8 + 0 .94 * log₂Pep-B1, | 1.52 | 3.77 | 100.36 ± 9.74 |
| | ratio (L/H); R\hat{ }2 = .998 | | | |
| Pep-A | log₂Con(ng/ml) = 8.72 + 0.67 * log₂Pep-A, | 6.12 | 12.40 | 100.46 ± 9.87 |
| | ratio (L/H); R\hat{ }2 = .998 | | | |
| Pep-CαK | log₂Con(ng/ml) = 7.118 + .942 * log₂Pep-Cαk, | 3.32 | 3.20 | 100.15 ± 5.46 |
| | ratio (L/H); R\hat{ }2 = .999 | | | |
| Pep-Cα | log₂Con(ng/ml) = 7.256 + .903 * log₂Pep-Cα, | 4.10 | 1.30 | 100.13 ± 5.36 |
| | ratio (L/H); R\hat{ }2 = .999 | | | |
| Pep-U1 | log₂Con(ng/ml) = 5.926 + .988 * log₂Pep-U1, | 1.01 | 1.14 | 100.13 ± 4.86 |
| | ratio (L/H); R\hat{ }2 = 1 | | | |
| Pep-U2 | log₂Con(ng/ml) = 4.43 + 1.046 * log₂ PepU2, | 0.20 | 1.56 | 103.25 ± 11.40 |
| | ratio (L/H); R\hat{ }2 = .998 | | | |
| Pep-U3 | log₂Conng/ml = 4.039 + 1.023 * log₂ Pep-U3, | 0.84 | 1.15 | 100.42 ± 9.48 |
| | ratio (L/H); R\hat{ }2 = .998 | | | |
| Pep-U4 | log₂Con(ng/ml) = 4.686 + 1.502 * log₂Pep-U4, | 0.12 | 1.23 | 98.92 ± 12.57 |
| | ratio (L/H); R\hat{ }2 = .998 | | | |
| Pep-U5 | log₂Con(ng/ml) = 3.099 + 1.056 * log₂Pep-U5, | 0.57 | 1.79 | 100.69 ± 12.22 |
| | ratio (L/H); R\hat{ }2 = .998 | | | |
| Pep-US | log₂Con, ng/ml = 6.87 + .944 * log₂Pep-US, | 14.46 | 2.04 | 114.21 ± 6.07 |
| | ratio (L/H); R\hat{ }2 = .999 | | | |
| Pep-UF | log₂Con(ng/ml) = 5.732 + .958 * log₂Pep-UF, | 7.09 | 9.91 | 100.25 ± 7.22 |
| | ratio (L/H); R\hat{ }2 = .998 | | | |

^{a} Linearity was determined by linear regression between measured concentration and peak area ratio (L/H) versus theoretical concentration determined by Light-SIS peptides (unlabeled form). All output of MultiQuant were weighted by 1/χ². Sum of all transition peak area ratio (L/H) to get peak area ratio at the peptide level.
^{b} LOQ was determined from the standard curve, defined as the lowest limits of quantification calibrated with acceptable CV <20% and accuracy within 100 ± 20%.
^{c} Accuracy was estimated by back fitting data to the STD curve and average recovery from all quantified points in the plots. Data are mean ± SD (%). Calibration curve is based on internal standard (IS unlabeled form) concentration and peak area ratio (transition ion peak area/IS peak area) and prepared in a pooled plasma matrix which was prepared from studying plasma samples >=5 points.

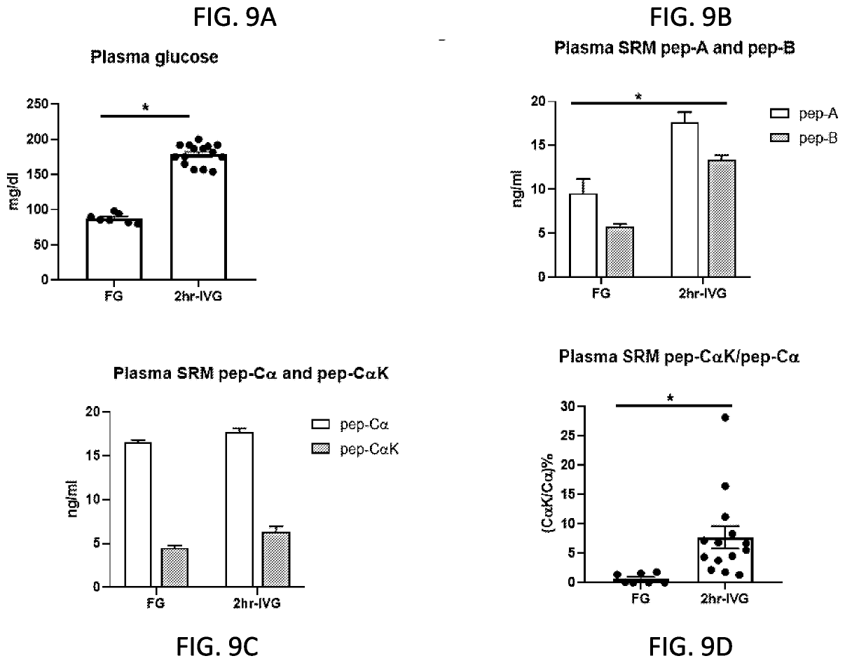

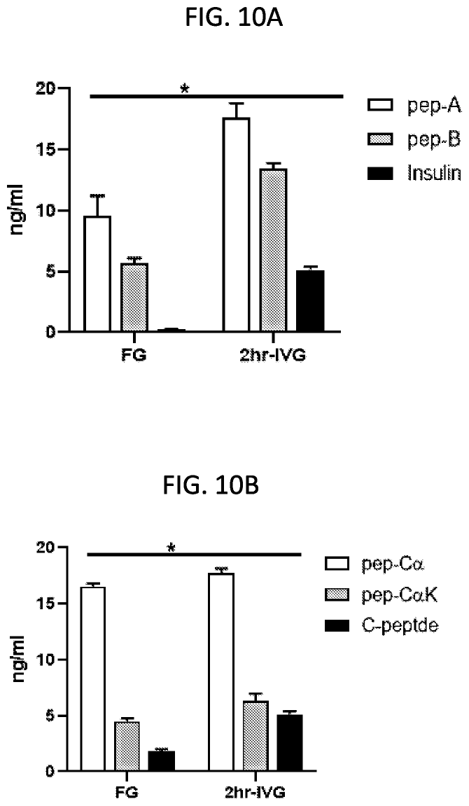

In order to check the robustness of the SRM-MS methodology for measuring translated products, as described above, A-chain, B-chain and Cα-peptide in plasma were measured under controlled conditions, after an intravenous hyperglycemic glucose clamp (IVG) during which circulating glucose levels were held constant at each subject's fasting level+98 mg/dl (all were non-diabetics). Fasting plasma glucose and after 2 hr-IVG were 88±2 and 179±4 mg/dl, respectively (FIG. 9A). There were significant increases after 2 hr-IVG, when measured by SRM, in both A- and B-chains (p<0.0001, FIG. 9B). The ELISA measures total insulin and, as expected, it was also significantly increased after 2 hr-IVG, in concordance with the SRM results (p<0.0001, FIG. 10A). The changes of pep-Cα (Cα) and pep-Cα K (CαK; a surrogate for 74-AA proinsulin) after 2 hr-IVG were much smaller (p=0.0579, FIG. 9C). However, there was a significant difference with ELISA measurement of C-peptide (p=0.002, FIG. 10B), demonstrating that Cα-peptide and 74-AA proinsulin were not as responsive to glucose as the classical C-peptide. The sample-matched ratio of CαK/Cα was calculated and it was found to be significantly increased after 2 hr-IVG (p=0.019, FIG. 9D), demonstrating that 2 hours after continuous hyperglycemia a significant amount of proinsulin, likely from immature granules, is released.

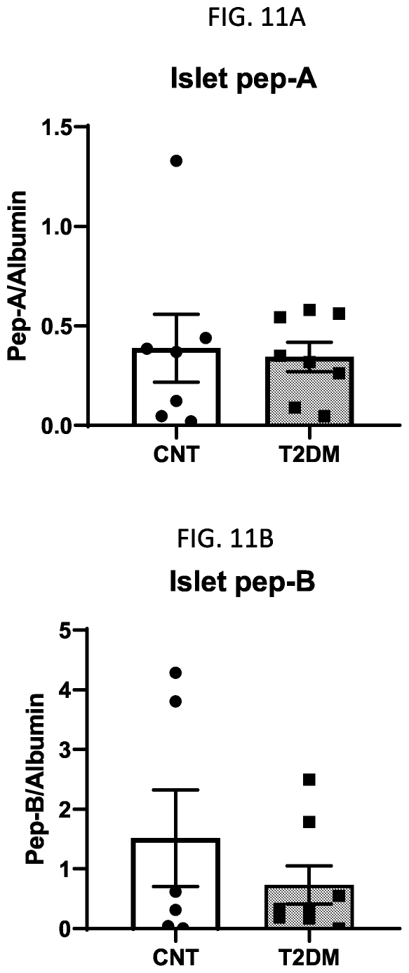

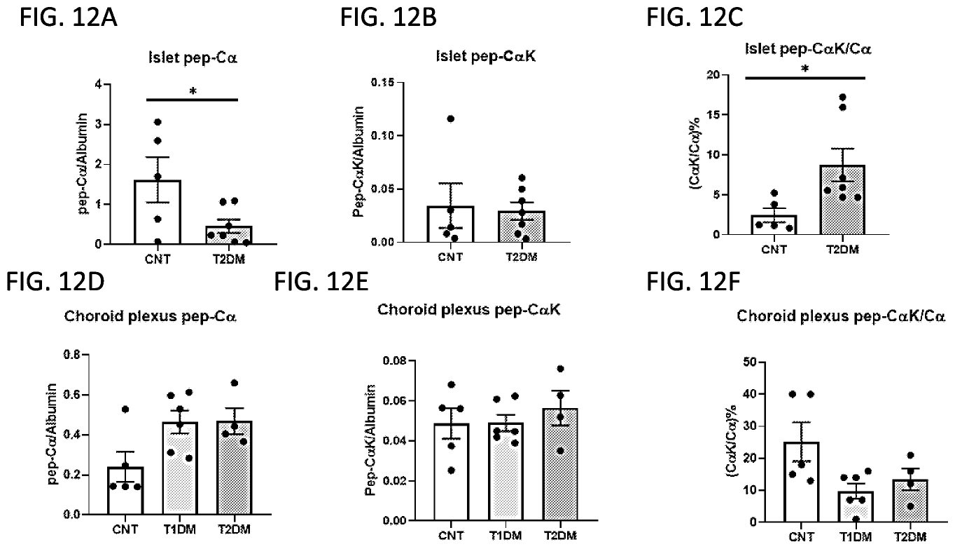

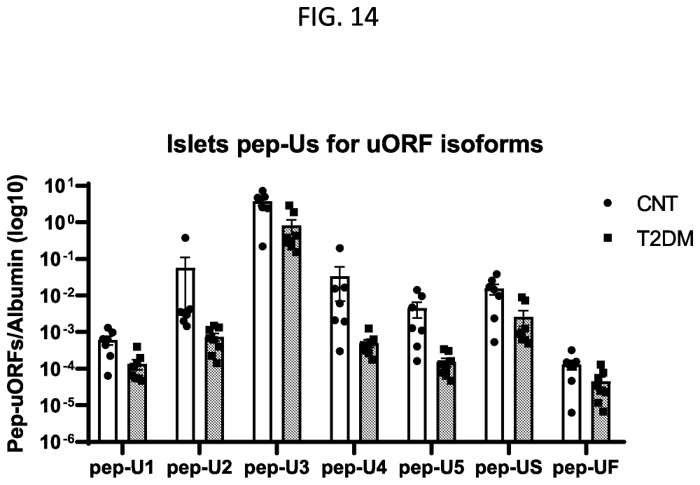

There were no significant changes in A- and B-chain levels extracted from islets between control and T2DM islets (FIGS. 11A and 11B). However, the amount of the Cα peptide was significantly lower in T2DM islets compared with control islets (p=0.047, FIG. 12A). The amounts of the 74-AA proinsulin represented by CαK were similar in control and T2DM islets (FIG. 12B), though the sample-matched ratio of CαK/Cα was significantly increased (p=0.035, FIG. 12C) in T2DM islets, implying proinsulin protease process is compromised in T2DM R cells. There were no significant changes in Cα, CαK, or ratio of CαK/Cα ratio in non-diabetic, T1DM, and T2DM CP samples (FIGS. 12D-12F). The SRM detection of the Cα-peptide again demonstrated insulin presence in postmortem CP individuals on life-long exogenous insulin and whose peripheral blood lacked C-peptide. Islet pepUs were also quantified in control and T2DM islets (FIG. 14).

### Example 7

### Exogenous Peptide-Assisted SRM (EPA-SRM) Proteomic Approach

In order to facilitate studies aimed at achieving further enhancement on SRM sensitivity without sacrificing throughput, an SRM approach, named exogenous peptide-assisted SRM (EPA-SRM) was developed for significantly improving quantitation accuracy of insulin α- and β-chains in plasma without adding any additional processing steps. EPA-SRM capitalizes on using precisely known concentrations of exogenous peptides as an adaptor to give low-abundant target analytes a better analyte-to-background ratio for eliminating potential co-eluting matrix effect in order to have maximum % of the measurement exceed limit of detection (LOD), at which the analyte can not only be reliably quantified but also some predefined goals for accuracy and imprecision (CV %) can be met. EPA-SRM provided further enhancement in SRM sensitivity and increased the overall sensitivity and accuracy in comparison to ELISA and standard SRM. Therefore, EPA-SRM should have broad application in accurate quantification of extremely low abundance but functionally important proteins in any biological sample in which matrix components interfere with SRM performance.

### Experimental Procedures

Development of SRM assay. The first step of SRM requires the development of assays and the selection of proteotypic peptides. Tryptic peptides were selected following the guidelines of Kuzyk and colleagues (Methods Mol. Biol. 1023:53-82, 2013). The selected peptides were synthesized as synthetic heavy-labeled and unlabeled analogues by Genemed Synthesis Inc. (San Antonio, TX, USA), and after reconstituting, each synthetic peptide concentration was determined by amino acid analysis (New England Peptide, Gardner, MA, USA). MS acquisition parameters require optimization and refinement by tuning acquisition in order to select the best transitions per a given peptide. Using synthetic peptides, optimal charge state, declustering potential (DP), collision energy (CE), collision cell exit potential (CXP), and interference detection are chosen, as detailed elsewhere (Zhang et al., Proteomics 17(6), 2017). Three to six interference-free precursor and fragment ion masses for a given peptide, called transitions, constituted the final SRM assay. For further enhancement of SRM sensitivity, the mass spectrometer was scheduled to collect subsets of peak based on the target analyte retention times (RT) on the column. Compared with classical SRM modes, the scheduled SRM provided better signal-to noise due to higher dwell times, and greatly improved reproducibility and accuracy by detecting more data points across chromatographic peaks. Details of SRM parameters are shown in Table 7.

TABLE 7
Selection of peptides and transitions optimized in scheduled
SRM for insulin peptides and human serum albumin (HSA) peptide

| | | | | Final SRM | |
|---|---|---|---|---|---|
| | MH+ | | Retention | Transitions | |
| Signature peptides | (mono) | Charges | time (min) | Q1 (Da) | Q3 (Da)^{b} |
| 169 INS β-chain2_GFFYTPK | 859.43 | 2 | 8.8 | 430.2 | 205.2, 244.5, |
| (SEQ ID NO: 71) | | | | | 344.9, 351.7, |
| | | | | | 508.2, 655.3 |
| 170 INS β-chain2_GFFYTPKA | 867.00 | 2 | 8.8 | 434.2 | 205.1, 252.2, |
| (SEQ ID NO: 71) | | | | | 353.2, 352.2, |
| | | | | | 516.3, 663.3 |
| 189 INS β-chain1_FVNQHLC | 2601.27 | 3 | 10.2 | 867.8 | 896.0, 1209.6, |
| (cam)GSHLVEALYLVC(cam)GERª | | | | | 602.2, 796.8, |
| (SQ ID NO: 54) | | | | | 733.2, 1080.3 |
| 190 INS β-chain1_FVANQHLC | 2609.00 | 3 | 10.2 | 870.4 | 896.4, 1209.6, |
| (cam)GSHLVEALYLVC(cam)GERª | | | | | 602.3, 800.9, |
| (SEQ ID NO: 54) | | | | | 733.3, 1080.5 |
| 175 INS α-chain_GIVEQC(cam) | 2611.09 | 3 | 10.4 | 871.3 | 698.9, 812.2, |
| C(cam)TSIC(cam)SLYQLENYC | | | | | 527.3 |
| (cam)N^{a} (SEQ ID NO: 55) | | | | | |
| 176 INS α-chain_GIV\hat{ }EQC(cam) | 2619.00 | 3 | 10.4 | 873.7 | 699.2, 812.3, |
| C(cam)TSIC(cam)SLYQLENYC | | | | | 535.3 |
| (cam)N^{a} (SEQ ID NO: 55) | | | | | |
| 1 HSA_LVNEVTEFAK (SEQ ID NO: | 1149.00 | 2 | 8.2 | 575.3 | 937.4, 694.3, |
| 70) | | | | | 823.5 |
| 2 HSA_LVNEVTEFAKA\hat{ } (SEQ ID | 1157.00 | 2 | 8.2 | 579.3 | 945.4, 702.3, |
| NO: 70) | | | | | 831.5 |

^{a}Cysteines are synthesized as carbamidomethyl (cam) cysteines
^{b}All SRM transitions were monitored and summed to determine LOQ, except for HSA

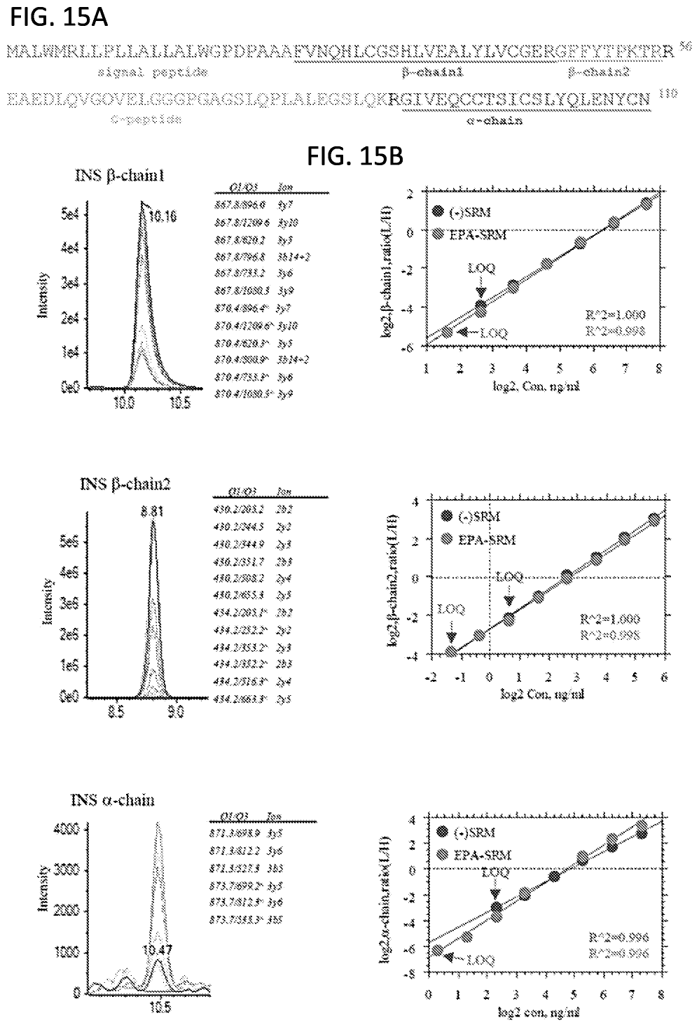

Sample preparation and measurements. Plasma insulin was measured in 22 healthy adults that underwent an oral glucose tolerance test (OGTT), in which 7 plasma samples were from 0 min (referred as fasting) and 15 samples from 2 hr. (referred as nonfasting) during OGTT. A pooled plasma was prepared from studying samples which were used for matrix-matched calibration curves and quality control (QC). All plasma samples were thawed on the day of analysis and centrifuged at 14,000×g for 15 min at 4° C. Cleared fractions were transferred with a loading tip into a fresh 1.5 ml polypropylene tube, discarding insoluble aggregates and the upper layer of floating lipids. Delipidated plasma was used parallel for both SRM and ELISA. For the ELISA a commercially available ELISA kit (Mercodia AB, Uppsala, Sweden) was used according to the manufacturer's recommendations. For SRM, an automated platform for sample dilution, denaturation, reduction, alkylation, trypsin digestion, and semi-automated solid-phase extraction of tryptic digest were conducted as described in detail elsewhere (Zhu et al., Proteomics 17(6), 2017). Due to the inherent instability of electrospray ionization, we added the heavy isotope-labeled peptide mixture (SISs) into the samples to achieve accurate and precise quantification. The SIS was a mixture of heavy-isotope labeled peptides synthesized with incorporated ¹⁵N and ¹³C isotopes corresponding to the targeted natural peptides. The dryness of tryptic digest was reconstituted with 80 μl of 0.1% formic acid and then equally divided into two parts, one of which was used for (−)SRM by adding an additional 10 μl of formic acid containing only SISs at final concentration of 100 fmol/μl, and the remaining part was used for EPA-SRM by adding 10 μl of 0.1% formic acid containing not only a SISs mixture at final concentration of 100 fmol/μl but also a mixture of exogenous peptides from peptide-β1 at final concentration of 15.6 ng/ml (6 fmol/μl), peptide-β2 at 15.9 ng/ml (18.5 fmol/μl) and for peptide-α at 15.7 ng/ml (6 fmol/μl), nearly 1:20 dilution for SRM analysis. Both natural and heavy isotope-labeled peptides were exactly coeluted with the target peptide in the chromatographic separation (FIG. 15). The SIS peptide spiking concentration was optimized to obtain an SRM signal for each peptide of about 10⁴ CPS (counts per second). Eight QC from a pooled plasma were prepared for the data quality control.

The scheduled SRM assay was run on a 5500 QTRAP (Sciex, Framingham, MA, USA) mass spectrometer equipped with an electrospray ionization source, a CBM-20A command module, and LC-20ADXR pump (Shimadzu Corp. Columbia, MD, USA) for solvent and sample delivery. A 2.1 mm×100 mm, 130 Å pore size, 3.5 m particle size C18 column (Waters Corp.) for the peptide separations using a linear gradient starting from 5% phase B increasing to 36% phase B within 10 min at a flow rate of 0.2 ml/min. Mobile phases consisted of water in 0.1% formic acid (phase A) and acetonitrile in 0.1% formic acid (phase B). The total run time was 18 min per sample. Triplicate injections of 10 μl of sample were carried out via the SIL-20AXR autosampler (Shimadzu Corp.). To eliminate possible carryover, the column was re-equilibrated at 50% B for 10 min and a blank run was performed prior to initiating the next sample injection. All sample data were collected using Analyst software version 1.6 and processed using MultiQuant software (version 3.02 with Scheduled-MRM-Algorithm) (Sciex) and each peak area was manually inspected to ensure correct peak detection and accurate integration. All outputs of MultiQuant were weighted by 1/χ².

Absolute quantitative SRM analysis. Calibration and characterization of limits of quantitation and variability are important aspects of any absolute quantitative assay (Azadeh et al., AAPS J 20(1):22, 2017). A comparative set of methods and approaches for EPA-SRM and (−)SRM assay calibration, regression analysis, dealing with natural signal, characterization of assay performance and precision were presented. For EPA-SRM and (−)SRM, a series of samples were analyzed that contained the sample matrix from the pooled plasma, a fixed concentration of SIS peptide (100 fmol/μl) and varied concentration of the analyte peptide (unlabeled SIS form) from 0.1 ng/ml to 1560 ng/ml by serial dilution that were added to each pooled plasma digest prior to solid-phase extraction (SPE) in which peptides were bound onto a solid support, interferences were washed off and peptides were eluted for further workup and SRM analysis. After SPE, the heavy-SIS peptide mixture (labeled form) at a final concentration of 100 fmol/μl was added to all digests including a blank digest. To differentiate from (−)SRM, an additional exogenous light-SIS peptide mixture at a precisely known concentration was added to each digest including a blank digest for EPA-SRM. These calibration standards provided similar overall composition between calibration curves and real sample matrix. A given peptide relative quantitation value was obtained by summing peak area ratio (light/heavy) from all target peptide transitions and then averaging over three technical runs. This should provide more robust results compared with individual transition, especially for low abundant peptides. Data are the binary logarithm transformed and plotted as measured concentration versus theoretical concentration for determining sample concentrations. The concentration spans of calibration curves were at least two orders of magnitude and bracket the lowest and upper limits of quantitation. All the calibration graphs with linear fitting correlation coefficient R² and LOQ are shown in FIG. 15B, right

### Results and Discussion

The amino acid sequence feature of human preproinsulin and peptide selection for the SRM analysis is shown in FIG. 15A. To validate EPA-SRM assay, specificity, linearity, LOQ, precision, and accuracy were evaluated and compared to those parameters of the (−)SRM assay. The specificity was assessed by confirming that there were no interference peaks in the plasma-matched matrix at the same retention time (FIG. 15B, left). On standard calibration curves, both EPA-SRM and (−)SRM quantitative response for 30 transitions representing the three signature insulin peptides (β1, β2 and α) had excellent linearity over 3-4 orders of magnitude (R²>=0.996) shown in FIG. 15B, right. The noise contributed by the sample matrix plays a major role in the magnitude of the calculated LOQ. As expected, with reduced noise signals by exogenous peptides, further enhancement of SRM sensitivity for all three insulin peptides was achieved by EPA-SRM, and, as a result, the LOQs were 3.05 ng/ml for β-chain1, 0.39 ng/ml for β-chain2 and 12.24 ng/ml for α-chain versus 6.01, 0.78 and 48.96 ng/ml for (−)SRM, respectively. At those concentration ranges, recoveries were nearly 100%, and CVs were <10% for both EPA-SRM and (−)SRM (Table 8).

TABLE 8
Comparison of quantification between EPA-SRM and (−)SRM assays

| | (−)SRM | | | | EPA-SRM | | | |
|---|---|---|---|---|---|---|---|---|
| Peptide | Linearity | LOQ | Recovery | | Linearity | LOQ | Recovery | |
| ID | (R²) | (ng/ml) | (%) | CV (%) | (R²) | (ng/ml) | (%) | CV (%) |
| INS β- | 1.000 | 6.096 | 99.97 ± 1.57 | 3.41 | 0.998 | 3.048 | 100.19 ± 6.84 | 2.19 |
| chain 1 | | | | | | | | |
| INS β- | 1.000 | 0.776 | 100.04 ± 3.10 | 2.14 | 0.998 | 0.388 | 100.27 ± 7.62 | 1.47 |
| chain 2 | | | | | | | | |
| INS α- | 0.996 | 4.900 | 100.28 ± 8.45 | 7.43 | 0.996 | 1.224 | 100.52 ± 10.85 | 9.21 |
| chain | | | | | | | | |

Notes:
1. LOQ, determined from the standard curve, defined as lowest limit of quantification calibrated with acceptable CV <20% and recovery within 100 ± 20%
2. Linearity, determined by linear regression between measured concentration-peak area ratio (L/H) versus theoretical concentration determined by light SIS peptides (unlabeled form)
3. Recovery, estimated by back fitting data to the standard curve
4. (−)SRM - without exogenous peptide assistance; EPA-SRM - with exogenous peptide assistance

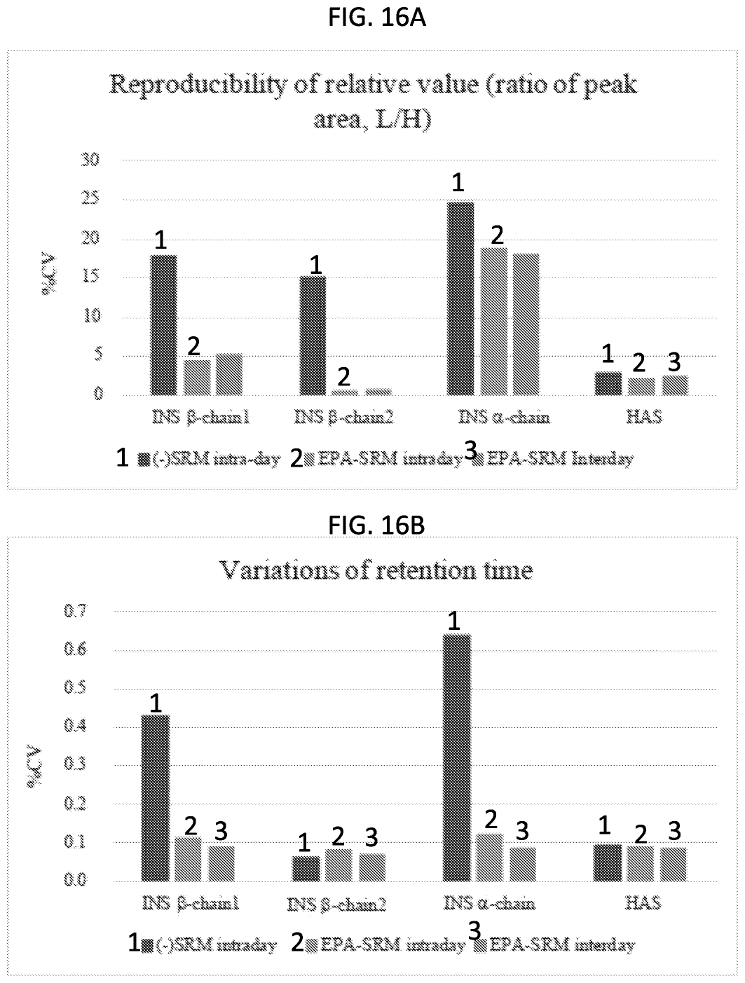

The reproducibility of EPA-SRM was evaluated by analysis of the entire SRM workflow replicates of the QC plasma sample either within a day (intra-assay) or a different day (inter-assay). As shown in FIG. 16A, the intra-day reproducibility of EPA-SRM was better than (−)SRM for all three peptides showing CV of 4.41% for β-chain1, 0.62% for β-chain2 and 18.85% for α-chain versus 17.89%, 15.08% and 24.64% for (−)SRM, respectively. This suggests that higher reproducibility was achieved by EPA-SRM. Notably, although % CV for α-chain had a 5.8% decrease by EPA-SRM, it was still higher than the expected 15%. This was possibly due to spiked amount of exogenous α-chain peptide (15.7 ng/ml) not being higher than the LOQ level (48.9 ng/mil) of the targeted analyte. Human serum albumin (HAS) was used here as the control peptide for monitoring day-to-day digestion processing and carryover from peptide separation column, which was constantly below 5% of CV. Retention time variations were monitored for system suitability (FIG. 16B). Retention time variations were less than 0.7% for all four peptides from both EPA-SRM and (−)SRM, indicating the conditions of chromatography were identical.

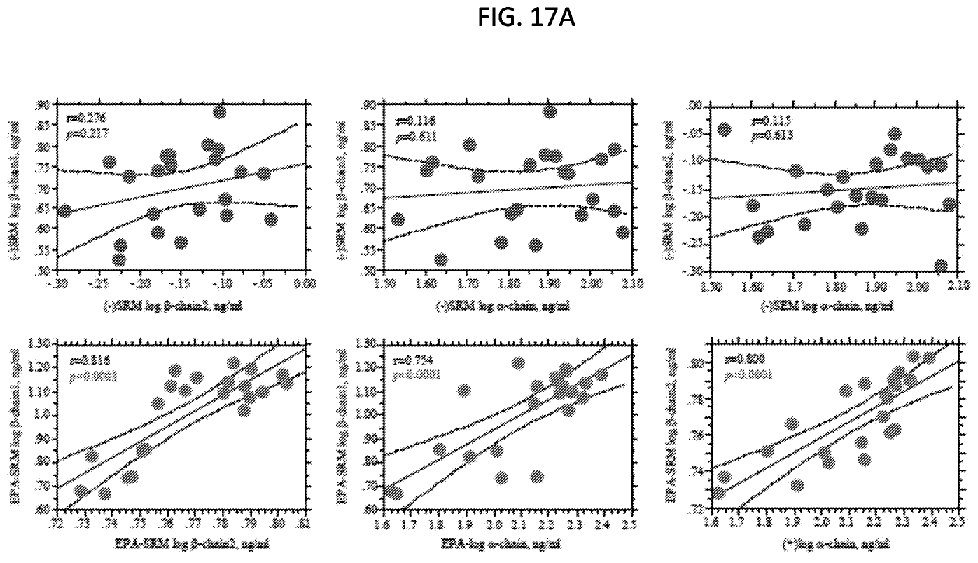

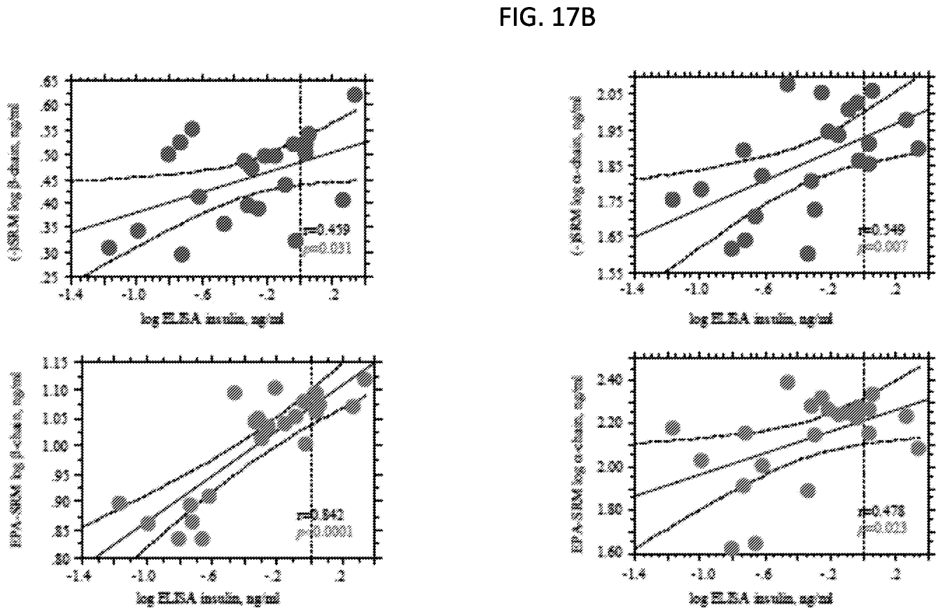

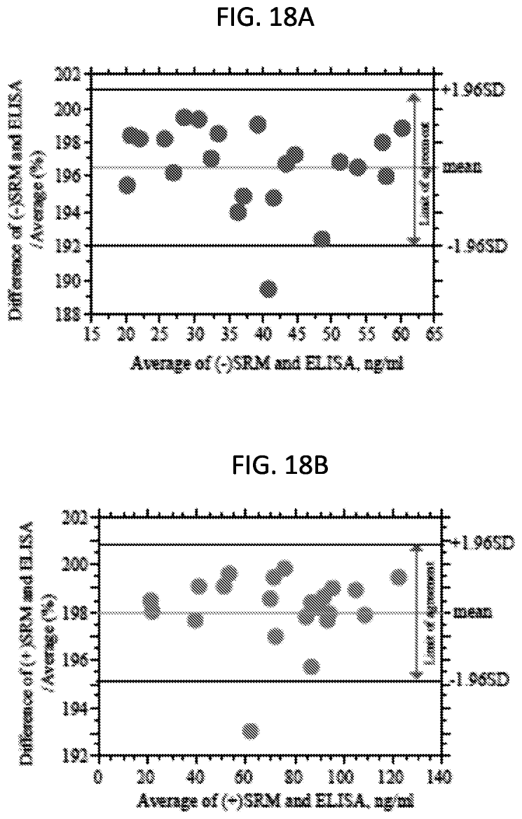

The determination of the sample accuracy is not often possible due to unknown concentrations of natural peptides in the samples, and therefore a comparison was used as a reference value with a commercially available ELISA kit. First, the Pearson correlation analysis was performed to determine whether an internal relationship between two sets of insulin peptides was present. After logarithmic transformation, Pearson correlation analysis revealed that quantification of those three peptides had significantly internal correlation for EPA-SRM (r>=0.75) but this internal relationship was not observed in (−)SRM (FIG. 17A). Since two β-chains had a good correlation (r=0.82, p<0.0001), we combined two β-chain quantitation as an average for the further analysis. To further confirm the accuracy of EPA-SRM measurement, results for plasma samples measured by SRM and ELISA were compared. As shown in FIG. 17B, significant correlation of absolute quantitation between EPA-SRM and ELISA were observed for β-chain (r=0.84, p<0.0001), but correlation between (−)SRM and ELISA was less desirable for β-chain (r=0.46, p=0.031), indicating the robustness of EPA-SRM analysis. However, there was no substantial improvement in quantitation accuracy of α-chain by EPA-SRM (r=0.48, p=0.023 versus 0.55, p=0.007 for (−)SRM), because exogenous α-chain peptide spiked was not higher than its LOQ level. This suggested that the exogenous peptide concentration used must be higher than the LOQ level of the target peptide. Since Pearson correlation coefficient (r) measures linear association rather than agreement between the methods being assessed (Altman et al. J. Roy Stat Soc D-Sta 32:307-317, 1983), Bland-Altman analysis was performed to determine whether the SRM quantitation agreed with ELISA results. As shown in FIGS. 18A and 18B, the differences between measurements against their average showed that both SRM and ELISA measurement were commutable. Combined with correlation analysis, results further demonstrated that EPA-SRM provided higher quantitation accuracy, and thus could be used for reliable low-abundant protein quantitation.

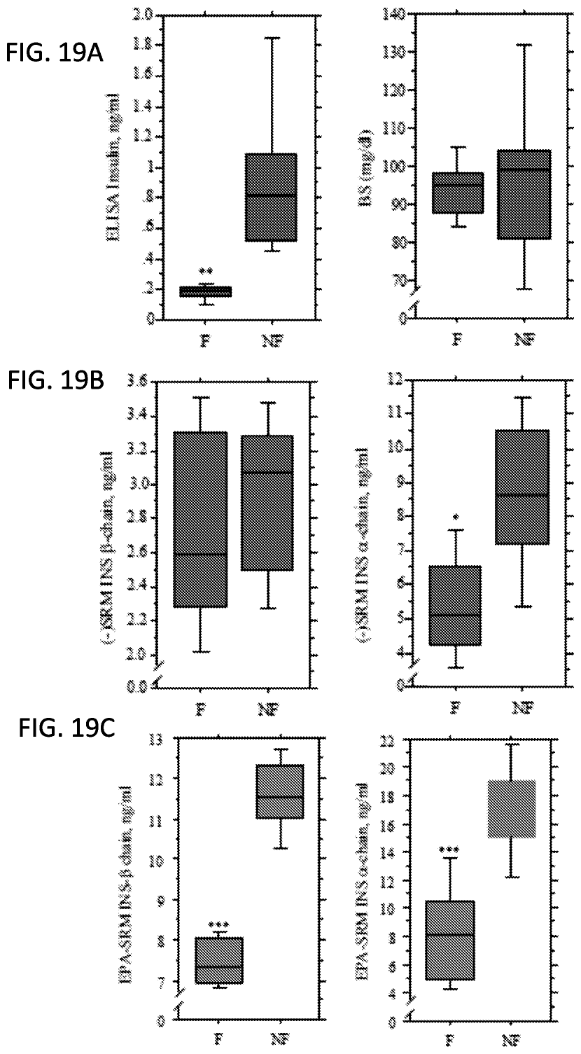

Finally, the relevance of SRM measurement in fasting and non-fasting states was underlined and compared the results from SRM to that from ELISA. As shown in FIG. 19, blood glucose had regained baseline level at 2 hrs after glucose ingestion. Plasma insulin was still significantly higher than fasting, as expected, based on the ELISA measurement (FIG. 19A). The concentrations of insulin β-chain and α-chain derived from EPA-SRM and (−)SRM were compared with ELISA assay. The EPA-SRM measurement was comparable to ELISA, results indicating that nonfasting plasma insulin was significantly higher than fasting plasma insulin (FIG. 19C). However, this significantly higher insulin level in nonfasting status versus fasting status did not become evident in (−)SRM assay (FIG. 19B). Based on EPA-SRM measurement, the mean concentration in the same set of samples was 10.28±2.1 ng/ml (peptide β) and 14.43±5.7 ng/ml (peptide α), respectively, versus 2.90±0.55 ng/ml (peptide β) and 7.59±2.60 ng/ml (peptide α) from (−)SRM and 0.70±0.55 ng/ml from ELISA (insulin). Results indicated that with EPA-SRM, the assay was able to detect 2-3 times higher insulin peptides than (−)SRM, probably due to reduced matrix effects and thereby maximizing percent of the measurement exceed their LOD levels produce analytical signals that meet predetermined targets for bias, imprecision and total error. It was also notable that EPA-SRM and ELISA approaches differ greatly in terms of absolute values of insulin in plasma. These discrepancies could be due to 1) SRM-based assays are not supported by an antigen/antibody reaction, but by a physical reaction, limiting affinity and specificity issues (Dupin et al., J. Proteome Res 15:2366-2378, 2016); 2) inherent properties of the peptide structure, its low immunogenicity, and its propensity to aggregate as well as the existence of smaller isoforms, which could affect the immunochemical assay results (Constance et al., Eupa Open Proteomics 3, 7, 2014); 3) inconsistency in the documented concentration of the standards used for SRM and ELISA. In immunoassays, it is assumed that a recombinant protein mimics the protein present in a tissue or a biofluid. In SRM, the peptides are synthesized, purified, and an accurate composition of amino acids is determined assuming resemblance with the peptide, which is a part of the target protein (Guzel et al., Proteomics Clin Appl. 12(1), 2018; Denburg et al., J. Bone Miner Res 31:1128-1136, 2016; Fu et al., Clin Chem. 62:198-207, 2016). Therefore, variations in the correct concentration of standards can be expected in these techniques; 4) a key aspect of SRM assay is that each targeted protein has multiple, partially independent, and layered observations which collectively indicate protein quantity (Fu et al., Quantitative Proteomics by Mass Spectrometry, 2^{nd} Edition 1410:249-264, 2016). This allowed independent quantification of a given protein by independent quantification of multiple transitions for the selected peptide(s). This differs from ELISA, where there is a single dependent measurement based on the binding of antibodies to two sites physically located within the protein, and 5) reduced antibody binding to endogenous analyte could be caused due to interference from unknown matrix components or structural modifications (e.g., posttranslational modifications, proteolysis) within analyte epitopes (Fu et al., Clin Chem. 62:198-207, 2016; Fu et al., Quantitative Proteomics by Mass Spectrometry, 2^{nd} Edition 1410:249-264, 2016). Furthermore, auto- or heterophilic antibodies present within the samples may cause biases in reported insulin concentrations as many diabetic patients treated with human or animal insulin develop anti-insulin antibodies (Vanhaeften, Diabetes Care 12:641-648, 1989). We also calculated the ratio of (3-chain/α-chain indicating 0.83 for EPA-SRM (closer to 1:1) versus 0.43 for (−)SRM. These results demonstrate that quantitative EPA-SRM yielded biological relevant results comparable to ELISA.

### Example 8

### Inhibition of IAPP Amyloid Fibrillation In Vitro

Thioflavin T (ThT) was from MilliporeSigma (catalog number T3516; Rockville, MD). IAPP (37 AA) (catalog number LT110006) (amidated C-terminal and disulfide bridge between Cys2 and Cys7) was from LifeTein (Hillsborough, NJ), and C-peptide (catalog number AS-61127) was from AnaSpec (Fremont, CA). Cα-peptide was custom made (Genemed Synthesis, Inc., San Antonio, TX). Black 96-well microplates (chimney well) were from Greiner Bio-One (Frickenhausen, Germany). Soluble and monomeric IAPP was made according to published protocols (Stine et al., Methods Mol. Biol. 670:13-32, 2011; Tu et al., Biochemistry 54:666-676, 2015), and the AnaSpec Manual of SensoLyte Thioflavin T Aggregation Kit (catalog number AS-72214) was used for the IAPP aggregation assay, with final concentration of 1% DMSO and 25 mmol/L ThT in component A buffer. The buffer was used as blank and IAPP without inhibitors, and the final concentration of IAPP and C- and Cα-peptides was 50 mmol/L each in the inhibition assays. Amyloid kinetics were measured by increasing ThT fluorescent amyloid binding intensity (1ex 440 nm; 1em 485 nm; height 3 mm; flashes 12) for 36 cycles, 5 min per cycle, at 37° C. with 15 s of shaking (100 rpm) between the reads in the EnSpire Multimode Plate Reader (PerkinElmer, Inc., Waltham, MA).

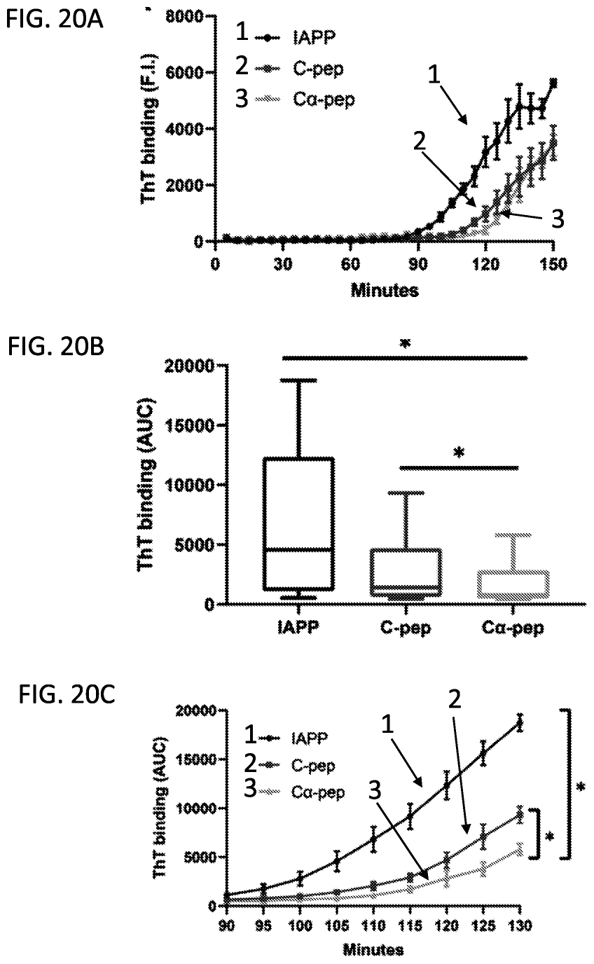

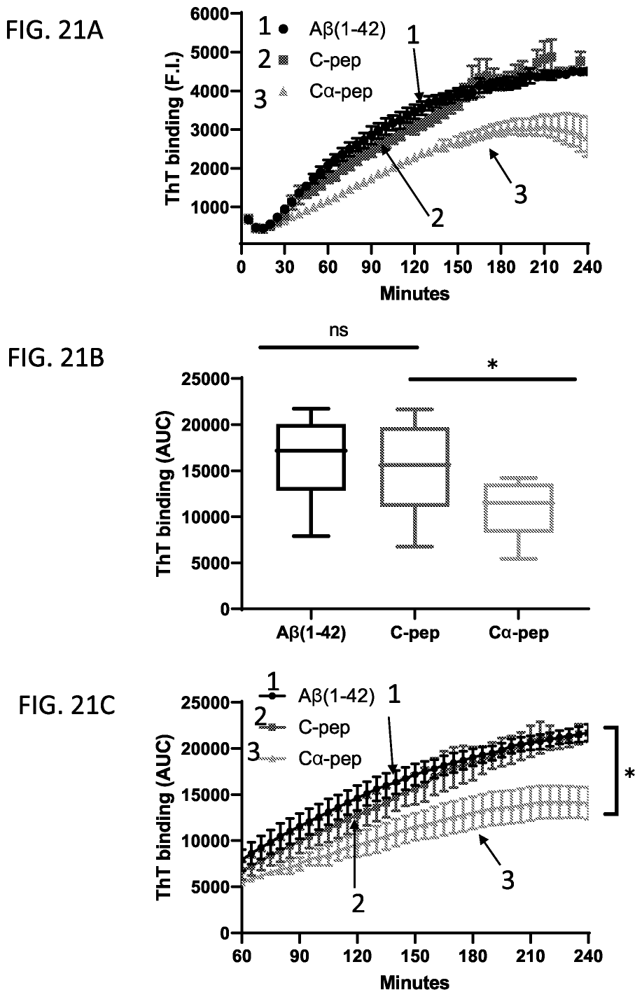

FIG. 20A is a representative experiment of three replicates showing IAPP fibrillation dynamics with lag time of 90 min. Cα-peptide inhibited IAPP fibrillation more efficiently than C-peptide by one-way ANOVA (P 5 0.032) (FIG. 20B) and by regression analysis (P 5 0.002) (FIG. 20C) in the range of 90-130 min. The ThT reporter was not affected by C- and Cα-peptides themselves (data not shown). Cα-peptide, but not C-peptide, also inhibits 0-amyloid fibrillation (FIGS. 21A-21C).

### Example 9

### Inhibition of Amyloidosis In Vivo

Inhibition of amyloidosis in vivo by Cα peptide is tested in rodent models of disease. Inhibition of IAPP fibrillation is tested in a diabetic human-IAPP (HIP) transgenic Sprague-Dawley rat model (Charles River Laboratory) that express human amylin on the rodent Ins2 promoter in the pancreatic β-cells (Despa et al., J. Am. Heart. Assoc. 3(4):e001015, 2014; Liu et al., Diabetes 65:2772-2783, 2016). The HIP rats develop diabetes and IAPP aggregation in islets by 5 months of age. Rats are administered 0.01 mg/kg to 200 mg/kg of Cα peptide or modified Cα peptide (such as cyclic, N-terminal acetylation-C-terminal amidation, or N-terminal fatty acid modification) intraperitoneally (i.p.) daily from age 3 months to 6 months. Reduction of islet IAPP amyloids is measured using Congo red staining of pancreas compared to vehicle i.p. control HIP rat groups.

Similarly, a transgenic mouse model of Alzheimer's Disease (2XTG AD mouse) whose genome contains human AD mutant genes of APPswe and PSEBldE9 (Hall et al., Brain Res. Bull. 88:3-12, 2012; Finnie et al., J. Comp. Pathol. 156:389-399, 2017) is used for testing inhibition of amyloid-β accumulation. The mouse brain develops amyloid fibrils at 4 months of age and peaks at 12 months. The mice are administered 0.01 mg/kg to 200 mg/kg of Cα peptide or modified Cα peptide (such as cyclic, N-terminal acetylation-C-terminal amidation, or N-terminal fatty acid modification) intraperitoneally (i.p.) starting at 4 months of age up to 12 months of age. Peptide (or vehicle) is administered daily. Reduction of Ab amyloids is measured using a mouse monoclonal antibody specific for Ab to monitor Ab fibrillation in comparison to vehicle i.p. control 2XTG AD mice.

In view of the many possible embodiments to which the principles of the disclosure may be applied, it should be recognized that the illustrated embodiments are only examples and should not be taken as limiting the scope of the invention. Rather, the scope of the invention is defined by the following claims. We therefore claim as our invention all that comes within the scope and spirit of these claims.

1. A method of treating a subject with diabetes or Alzheimer's disease or inhibiting amyloidosis in a subject, comprising administering to the subject a composition comprising an insulin Cα peptide to a subject in need thereof.

2. (canceled)

3. The method of claim 1, wherein the amyloidosis is pancreatic islet amyloidosis or brain amyloidosis.

4. The method of claim 1, wherein the amino acid sequence of the insulin Cα peptide comprises or consists of the amino acid sequence of SEQ ID NO: 7 or SEQ ID NO: 8.

5. (canceled)

6. The method of claim 1, wherein the Cα peptide comprises one or more modifications.

7. The method of claim 6, wherein the one or more modifications is selected from N-terminal acetylation, C-terminal amidation, PEGylation, cyclization, a C14-C18 fatty acid, caprylic acid, fusion with vitamin B12, and a combination of two or more thereof.

8. The method of claim 1, wherein the subject has type 1 diabetes or type 2 diabetes.

9. The method of claim 8, further comprising administering to the subject one or more of an insulin, a biguanide, a thiazolidinedione, a sulfonylurea, an incretin, and a sodium glucose co-transporter 2 inhibitor.

10. The method of claim 1, wherein the subject has Alzheimer's disease.

11. The method of claim 1, wherein the composition comprising the insulin Cα peptide is administered parenterally, orally, or via a patch.

12. A set of one or more peptides selected from the group consisting of the amino acid sequences of SEQ ID NOs: 54-64.

13. The set of peptides of claim 12, wherein one or more of the peptides comprises carbamidomethylation of one or more cysteine residues and/or an isotope label on one or more amino acids.

14. (canceled)

15. The set of peptides of claim 12, wherein the set of peptides comprises each of SEQ ID NOs: 54-64.

16. A method of detecting presence or amount of one or more insulin isoforms or Cα peptides in a sample, comprising:
treating the sample with one or more enzymes to digest proteins in the sample;
desalting the treated sample;
adding the set of one or more peptides of claim 12; and
analyzing the treated sample by tandem mass spectrometry.

17. The method of claim 16, wherein treating the sample with one or more enzymes to digest proteins comprises treating the sample with trypsin.

18. The method of claim 16, wherein the tandem mass spectrometry is liquid chromatography-mass spectrometry-mass spectrometry.

19. The method of claim 16, wherein the sample is a biological sample from a subject.

20. The method of claim 19, wherein the biological sample is a blood, plasma, serum, urine, or tissue sample.

21. The method of claim 19, wherein the subject has, is suspected to have, or is at risk of having pre-diabetes, diabetes, or Alzheimer's disease.

22. The method of claim 21, further comprising identifying the subject as having type 2 diabetes if the amount of Cα peptide in the sample is increased compared to a control.

23. The method of claim 22, further comprising administering to the subject one or more of an insulin Cα peptide, an insulin, a biguanide, a thiazolidinedione, a sulfonylurea, an incretin, or a sodium glucose co-transporter 2 inhibitor.

24. The method of claim 21, further comprising identifying the subject as having Alzheimer's disease if the amount of INSU2 (SEQ ID NO: 2) is increased compared to a control.

25. The method of claim 24, further comprising administering to the subject one or more of an insulin Cα peptide, galantamine, rivastigmine, donepezil, memantine, and aducanumab.

26. A modified insulin Cα peptide comprising or consisting of the amino acid sequence of SEQ ID NO: 7 or SEQ ID NO: 8 and one or more peptide modifications.

27. (canceled)

28. The modified insulin Cα peptide of claim 26, wherein the one or more peptide modifications is selected from N-terminal acetylation, C-terminal amidation, PEGylation, cyclization, a C14-C18 fatty acid, caprylic acid, fusion with vitamin B12, and a combination of two or more thereof.


SEQUENCE LISTING (71 sequences)

SEQ ID NO: 1 - protein, 204 aa, Homo sapiens
MGSETIKPAGAQQPSALQDRLHQKRPSSRSVPRAFASGGLRIPGWLDPRPQLCSREDVAGLVKHVGVSPGAPRQGTWPSACLSPACLPDHCPSAMALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 2 - protein, 198 aa, Homo sapiens
MGSETIKPAGAQQPSALQDRLHQKRPSSRSVPRAFASGGLRIPGWLDPRPQLCSREDVAGLVKHVGVSPGAPRQGTWPSACLSPACLPDHCPSAMALWMRLLPLLALLALWGPDPAAAFVNQHLCGSHLVEALYLVCGERGFFYTPKTRREAEDLQGEPTAHCCPWPPPATPCSWRSHPAWAEGGRRLPPSRGSGALF

SEQ ID NO: 3 - protein, 53 aa, Homo sapiens
MGSETIKPAGAQQPSALQDRLHQKRPSSRSLSFCHGPVDAPPAPAGAAGPLGT

SEQ ID NO: 4 - protein, 153 aa, Homo sapiens
MGSETIKPAGAQQPSALQDRLHQKRPSSRSVPRAFASDHCPSAMALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 5 - protein, 73 aa, Homo sapiens
MGSETIKPAGAQQPSALQDRLHQKRPSSRSVPRAFASGGLRIPGWLDPRSLSFCHGPVDAPPAPAGAAGPLGT

SEQ ID NO: 6 - protein, 98 aa, Homo sapiens
MALWMRLLPLLALLALWGPDPAAAFVNQHLCGSHLVEALYLVCGERGFFYTPKTRREAEDLQGSLQPLALEGSLQKRGIVEQCCTSICSLYQLENYCN

SEQ ID NO: 7 - protein, 19 aa, Homo sapiens
EAEDLQGSLQPLALEGSLQ

SEQ ID NO: 8 - protein, 20 aa, Homo sapiens
EAEDLQGSLQPLALEGSLQK

SEQ ID NO: 9 - DNA, 18 bp, Artificial Sequence
  INS EX2 probe
tgaaccaacacctgtgcg

SEQ ID NO: 10 - DNA, 16 bp, Artificial Sequence
  INS E2 forward primer
cccagccgcagccttt

SEQ ID NO: 11 - DNA, 22 bp, Artificial Sequence
  INS EX2 reverse primer
agagagcttccaccaggtgtga

SEQ ID NO: 12 - DNA, 20 bp, Artificial Sequence
  INS EX2-12 probe
acgaggcttcttctacacac

SEQ ID NO: 13 - DNA, 22 bp, Artificial Sequence
  INS EX2-12 forward primer
tcacacctggtggaagctctct

SEQ ID NO: 14 - DNA, 18 bp, Artificial Sequence
  INS EX2-12 reverse primer
ggcagcaatgggcagttg

SEQ ID NO: 15 - DNA, 19 bp, Artificial Sequence
  INS INS1A probe
ccatcaagcagatcactgt

SEQ ID NO: 16 - DNA, 21 bp, Artificial Sequence
  INS INS1A forward primer
ggacaggctgcatcagaagag

SEQ ID NO: 17 - DNA, 18 bp, Artificial Sequence
  INS INS1A reverse primer
acagggccatggcagaag

SEQ ID NO: 18 - DNA, 16 bp, Artificial Sequence
  INS INS1B probe
tttgcgtcagatcact

SEQ ID NO: 19 - DNA, 21 bp, Artificial Sequence
  INS INS1B forward  primer
ccatcaagcaggtctgttcca

SEQ ID NO: 20 - DNA, 17 bp, Artificial Sequence
  INS INS1B reverse primer
gggccatggcagaagga

SEQ ID NO: 21 - DNA, 16 bp, Artificial Sequence
  INS INS1C probe
accccagatcactgtc

SEQ ID NO: 22 - DNA, 21 bp, Artificial Sequence
  INS INS1C forward primer
ccatcaagcaggtctgttcca

SEQ ID NO: 23 - DNA, 16 bp, Artificial Sequence
  INS INS1C reverse primer
gcaggaggcgcatcca

SEQ ID NO: 24 - DNA, 16 bp, Artificial Sequence
  INS INSI1 priobe
ccatcaagcaggtctg

SEQ ID NO: 25 - DNA, 21 bp, Artificial Sequence
  INS INSI1 forward primer
ggacaggctgcatcagaagag

SEQ ID NO: 26 - DNA, 18 bp, Artificial Sequence
  INS INSI1 reverse primer
agcccacctgacgcaaag

SEQ ID NO: 27 - DNA, 19 bp, Artificial Sequence
  INS INSUA probe
ccatcaagcagatcactgt

SEQ ID NO: 28 - DNA, 24 bp, Artificial Sequence
  INS INSUA forward primer
gggagatgggctctgagactataa

SEQ ID NO: 29 - DNA, 18 bp, Artificial Sequence
  INS INSUA reverse primer
acagggccatggcagaag

SEQ ID NO: 30 - DNA, 16 bp, Artificial Sequence
  INS INSUB probe
tttgcgtcagatcact

SEQ ID NO: 31 - DNA, 24 bp, Artificial Sequence
  INS INSUB forward primer
gggagatgggctctgagactataa

SEQ ID NO: 32 - DNA, 17 bp, Artificial Sequence
  INS INSUB reverse primer
gggccatggcagaagga

SEQ ID NO: 33 - DNA, 16 bp, Artificial Sequence
  INS INSUC probe
accccagatcactgtc

SEQ ID NO: 34 - DNA, 24 bp, Artificial Sequence
  INS INSUC forward primer
gggagatgggctctgagactataa

SEQ ID NO: 35 - DNA, 16 bp, Artificial Sequence
  INS INSUC reverse primer
gcaggaggcgcatcca

SEQ ID NO: 36 - DNA, 16 bp, Artificial Sequence
  INS INSU1 probe
ccatcaagcaggtctg

SEQ ID NO: 37 - DNA, 24 bp, Artificial Sequence
  INS INSU1 forward primer
gggagatgggctctgagactataa

SEQ ID NO: 38 - DNA, 18 bp, Artificial Sequence
  INS INSU1 reverse primer
agcccacctgacgcaaag

SEQ ID NO: 39 - DNA, 15 bp, Artificial Sequence
  INS INS3B probe
caggctgccctgcag

SEQ ID NO: 40 - DNA, 23 bp, Artificial Sequence
  INS INS3B forward primer
tagagggagcagatgctggtaca

SEQ ID NO: 41 - DNA, 23 bp, Artificial Sequence
  INS INS3B reverse primer
aggcttcttctacacacccaaga

SEQ ID NO: 42 - DNA, 19 bp, Artificial Sequence
  Ins Ins2-V1 probe
caagcaggaaggttattgt

SEQ ID NO: 43 - DNA, 22 bp, Artificial Sequence
  Ins Ins2-V1 forward primer
ccgctacaatcaaaaaccatca

SEQ ID NO: 44 - DNA, 17 bp, Artificial Sequence
  Ins Ins2-V1 reverse primer
catccacagggccatgt

SEQ ID NO: 45 - DNA, 17 bp, Artificial Sequence
  Ins Ins2-V2 probe
caagcaggaaggtactc

SEQ ID NO: 46 - DNA, 22 bp, Artificial Sequence
  Ins Ins2-V2 forward primer
ccgctacaatcaaaaaccatca

SEQ ID NO: 47 - DNA, 18 bp, Artificial Sequence
  Ins Ins2-V2 reverse primer
gagccaggcccactgaga

SEQ ID NO: 48 - DNA, 15 bp, Artificial Sequence
  Ins Ins2-V3 probe
atccgctacaatcaa

SEQ ID NO: 49 - DNA, 22 bp, Artificial Sequence
  Ins Ins2-V3 forward primer
ccagtaaccaccagccctaagt

SEQ ID NO: 50 - DNA, 24 bp, Artificial Sequence
  Ins Ins2-V3 reverse primer
agataggcttcctgcttgctgatg

SEQ ID NO: 51 - DNA, 19 bp, Artificial Sequence
  Ins Ins2-V4 probe
ctggaggttattgtttcaa

SEQ ID NO: 52 - DNA, 21 bp, Artificial Sequence
  Ins Ins2-V4 forward primer
gctcctacgctgaaattccaa

SEQ ID NO: 53 - DNA, 21 bp, Artificial Sequence
  Ins Ins2-V4 reverse primer
aaggtgctgcttgacaaaagc

SEQ ID NO: 54 - protein, 22 aa, Artificial Sequence
  Insulin tryptic peptide pep-B
FVNQHLCGSHLVEALYLVCGER

SEQ ID NO: 55 - protein, 21 aa, Artificial Sequence
  Insulin tryptic peptide pep-A
GIVEQCCTSICSLYQLENYCN

SEQ ID NO: 56 - protein, 19 aa, Artificial Sequence
  Insulin tryptic peptide pep-Calpha
EAEDLQGSLQPLALEGSLQ

SEQ ID NO: 57 - protein, 20 aa, Artificial Sequence
  Insulin tryptic peptide pep-CalphaK
EAEDLQGSLQPLALEGSLQK

SEQ ID NO: 58 - protein, 13 aa, Artificial Sequence
  Insulin tryptic peptide pep-U1
PAGAQQPSALQDR

SEQ ID NO: 59 - protein, 8 aa, Artificial Sequence
  Insulin tryptic peptide pep-U2
AFASGGLR

SEQ ID NO: 60 - protein, 8 aa, Artificial Sequence
  Insulin tryptic peptide pep-U3
IPGWLDPR

SEQ ID NO: 61 - protein, 8 aa, Artificial Sequence
  Insulin tryptic peptide pep-U4
EDVAGLVK

SEQ ID NO: 62 - protein, 10 aa, Artificial Sequence
  Insulin tryptic peptide pep-U5
HVGVSPGAPR

SEQ ID NO: 63 - protein, 16 aa, Artificial Sequence
  Insulin tryptic peptide pep-US
AFASDHCPSAMALWMR

SEQ ID NO: 64 - protein, 10 aa, Artificial Sequence
  Insulin tryptic peptide pep-UF
SHPAWAEGGR

SEQ ID NO: 65 - DNA, 12 bp, Artificial Sequence
  Alternative consensus Kozak ribosomal binding site
tgggagatgggc

SEQ ID NO: 66 - protein, 19 aa, Artificial Sequence
  C-alpha peptide
EAEDLQGSLQPLALEGSLQ

SEQ ID NO: 67 - protein, 5 aa, Artificial Sequence
  Beta sheet forming motif
GQVEL

SEQ ID NO: 68 - protein, 14 aa, Artificial Sequence
  Instrinsically disordered protein region sequence
VELGGGPGAGSLQP

SEQ ID NO: 69 - protein, 110 aa, Homo sapiens
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 70 - protein, 10 aa, Artificial Sequence
  Human serum albumin tryptic peptide
LVNEVTEFAK

SEQ ID NO: 71 - protein, 7 aa, Artificial Sequence
  Insulin tryptic peptide
GFFYTPK